(19) United States
(12) Patent Application Publication
(10) Pub. No.: US 2026/0327273 A1
(43) Pub. Date: Oct. 22, 2026

(54) ADP-ACTIN-SPECIFIC PROTEIN AND MOTIFS TO REMODEL ACTIN CYTOSKELETON IN LIVING CELLS AND IN VITRO BIOCHEMISTRY

(71) Applicant: Nanyang Technological University, Singapore (SG)

(72) Inventors: Yansong Miao, Singapore (SG); Qianqian Ma, Singapore (SG); Danxia He, Singapore (SG); Cai Xin Ng, Singapore (SG)

(73) Assignee: Nanyang Technological University, Singapore (SG)

(21) Appl. No.: 19/101,208

(22) PCT Filed: Aug. 4, 2023
(86) PCT No.: PCT/SG2023/050540

§ 371 Date: Feb. 4, 2025

(30) Foreign Application Priority Data
Aug. 5, 2022 (SG) 10202250671T

(51) Int. Cl. C07K 14/395; A61K 38/00; G01N 33/50

(52) U.S. Cl. CPC C07K 14/395; G01N 33/502; A61K 38/00; C07K 2319/60; G01N 2333/395

(57) ABSTRACT

The present invention generally relates to recombinant polypeptides capable of regulating actin remodelling. In particular, the polypeptides disclosed herein are selective for ADP-actin and may modulate ADP-actin remodelling under energy starvation conditions. The recombinant polypeptides of the present invention are also suitable for use in detecting ADP-actin activity in vitro or in living cells, in drug screening, in therapeutic approaches and for the treatment of actin-associated diseases.

### FIELD OF THE INVENTION

The present invention generally relates to recombinant polypeptides capable of regulating actin remodelling. In particular, the polypeptides disclosed herein are selective for ADP-actin and may modulate ADP-actin remodelling under energy starvation conditions. The recombinant polypeptides of the present invention are also suitable for use in detecting ADP-actin activity in vitro or in living cells, in drug screening, in therapeutic approaches and for the treatment and prophylaxis of actin-associated diseases.

### BACKGROUND OF THE INVENTION

Actin is highly conserved and is one of the most abundant proteins on earth. Actin participates in a variety of biological activities such as cell division, intracellular transport, morphogenesis, and muscle contraction, and is essential for the survival of most cells by influencing eukaryotic cell shape and behaviour. In this regard, monomeric actins polymerize into filaments and is then assembled into filamentous networks by going through different steps of actin polymerization from initial nucleation, capping, elongation, crosslinking, and depolymerization (Pollard, T. D., and Cooper, J. A., Science 326, 1208-1212 (2009)). Actin-binding proteins (ABPs) on the other hand, are known to regulate the organization and dynamics of the actin cytoskeleton. Many ABPs function at different steps of actin polymerization and depolymerization to coordinate the appropriate assembly speed and network shapes. ABPs regulate at almost every stage of the actin filament assembly and disassembly cycles, and control how the actin filaments are arranged into various three-dimensional configurations as well.

The remodelling of the actin cytoskeleton during diverse signaling events is sophisticated, and the underlying mechanisms remain elusive. Dynamic treadmilling and network organization of the actin cytoskeleton rely on the orchestrated operation of different ABPs in an actin nucleotide-dependent fashion. While the structures of ATP- and ADP-actin are nearly identical, actin can also adopt dynamic conformations. The nucleotide state is one of the major factors that can control actin conformation via allosteric communication between nucleotide binding sites and their spatially distinct regions (Ali, R. et al., Nature structural & molecular biology 29, 320-328 (2022)). Whereas the nucleotide state can modulate actin nucleation by creating a nucleation-potent conformation, nucleotide-specific binding by actin-binding proteins also plays critical roles in the rapid influence on actin treadmilling and the timely remodelling of the actin cytoskeleton. In the past, nucleation, elongation and crosslinking were mostly studied only in the presence of ATP but remained unclear for ADP-actin, which could be a dominant state during cell signaling, such as under energy starvation(ES).

Undesired environments often lead to energy starvation(ES) in eukaryotic cells, which limits growth by interrupting diverse active-running ATP-dependent processes. Under ES, cells enter a dormant state as an adaptation strategy before returning to a normal cell state to survive harsh conditions. Glucose starvation (GS), which is a major physiological ES condition, has been found to remodel actin filaments in budding yeast acutely within a few minutes by converting G-to F-actin and inducing a rapid stabilization of actin cables that are resistant to cofilin-mediated severing (Xu, L., and Bretscher, A., Curr Biol 24, 2471-2479 (2014); Atkinson, S. J. et al., J Biol Chem 279, 5194-5199 (2004)). Actin cable treadmilling was orchestrated spatiotemporally by a series of ABPs to drive stepwise polymerization and depolymerization steps, coordinating with the nucleotide exchange of actin. GS-induced ATP-depleting conditions interrupted the turnover rate of ATP hydrolysis and inorganic phosphate (Pi) release and increased the ADP: ATP ratio, thereby leading to faster aging of the actin filament from ATP to ADP subunits. Strikingly, instead of being depolymerized by ADP-actin-based depolymerization factors, GS triggers actin cable crosslinking to stabilize F-actin and retain the mass of actin networks. Earlier genetic screening found that GS-triggered actin cable bundling is independent of two cable-binding proteins, type V myosin and tropomyosin (Xu, L., and Bretscher, A., Curr Biol 24, 2471-2479 (2014)). However, the underlying mechanism remains unknown, and the ABPs responsible for stabilizing ADP-F-actin have not been identified.

As observed, ADP-actin-specific regulators are poorly understood, particularly in cell signaling. Therefore, targeting ADP-actin specific modulators may provide a potential approach in the study of actin dynamics, in cell-based drug screenings and in the provision of therapeutics for the treatment of actin-associated diseases.

Accordingly, there is a need to provide ADP-actin targeting peptides that overcome or at least ameliorate, one or more of the drawbacks described above.

### SUMMARY OF THE INVENTION

The present invention relates to the provision of recombinant polypeptides capable of modulating ADP-actin remodelling and preferably, regulating ADP-actin remodelling induced under energy starvation. The recombinant polypeptides disclosed herein are also suitable for use in drug screening and for therapeutic applications in the treatment of actin-related diseases.

In a first aspect, there is provided a recombinant polypeptide comprising an amino acid sequence of at least 85%, at least 90%, at least 95%, at least 96%, at least 97%, at least 98%, or at least 99% identity to the amino acid sequence set forth in SEQ ID NO: 1 or SEQ ID NO: 2, or a functional fragment or a functional variant thereof, wherein the polypeptide, fragment or variant thereof specifically binds to ADP-actin and modulates ADP-actin remodelling. In this regard, the recombinant polypeptides disclosed herein preferably modulate ADP-actin remodelling by enhancing ADP-actin nucleation, elongation/polymerization, crosslinking, bundling, stabilization and/or inhibition of actin depolymerization. In some embodiments, the ADP-actin remodelling is induced under energy starvation conditions, such as glucose starvation conditions.

In a second aspect, there is provided a polynucleic acid molecule, wherein said polynucleic acid molecule has a sequence of at least 85%, at least 90%, at least 95%, at least 96%, at least 97%, at least 98%, or at least 99% identity to the polynucleic acid sequence set forth in SEQ ID NO: 8 or in SEQ ID NO: 9, or a fragment thereof, which encodes the polypeptide of the first aspect or its functional fragment or functional variant thereof.

In a third aspect, there is provided a recombinant cell transfected or transformed with the polynucleic acid molecule of the second aspect.

In a fourth aspect, there is provided a method for the detection and/or modulation of ADP-actin remodelling, or in drug screening of candidate modulators of ADP-actin remodelling may comprise (i) expressing a tagged recombinant polypeptide of the first aspect in a cell; (ii) detecting whether the tagged polypeptide associates with ADP-actin or F-actin using total internal reflection (TIRF) microscopy; and for drug screening, exposing the cell before step (ii) to candidate modulators of ADP-actin remodelling. Preferably, the method is in vitro.

In a fifth aspect, there is provided a pharmaceutical composition comprising a recombinant polypeptide of the first aspect and a pharmaceutically acceptable carrier.

In a sixth aspect, there is provided a recombinant polypeptide of the first aspect, a polynucleic acid molecule of the second aspect, or a recombinant cell of the third aspect for use in medicine.

In a seventh aspect, there is provided a use of a recombinant polypeptide of the first aspect, a polynucleic acid molecule of the second aspect, or a recombinant cell of the third aspect, in cell-based screens for drugs or in the study of diseases associated with actin-remodelling, wherein the use is not in a human being.

In an eighth aspect, there is provided a method of treating a disease associated with actin-remodelling, comprising administering a therapeutically effective amount of a recombinant polypeptide of the first aspect or a pharmaceutical composition of the fifth aspect, to a patient in need thereof.

In a ninth aspect, there is provided a use of a recombinant peptide according to the first aspect, or a pharmaceutical composition of the fifth aspect, in the manufacture of a medicament for the treatment of a disease associated with actin-remodelling. A person skilled in the art would understand that a disease associated with actin-remodelling that may be treated by the invention may include myopathies, familial thoracic aortic aneurysms and heart diseases, being related to actin mutation. There are reports indicating that Baraitser-Winter syndrome is caused by Met47 to Thr, and this amino acid is located in the actin D-loop. More particularly, the disease associated with actin-remodelling is a disease associated with ADP-actin remodelling.

Advantageously, the polypeptides disclosed herein may target different stages in the actin remodelling cycle and act as multifunctional ADP-actin modulators. These and other advantages of the present disclosure will become readily apparent to those skilled in this art from the following detailed description.

### BRIEF DESCRIPTION OF THE FIGURES

The accompanying drawings illustrate disclosed embodiments and serve to explain the principles of the disclosed embodiments. It is to be understood, however, that the drawings are designed for purposes of illustration only, and not as a definition of the limits of the invention.

FIG. 1 shows that Spa2 regulates actin cable remodelling upon energy starvation. Representative maximum Z-projection images (A) and quantification of ABP140-3XGFP signals (B) in WT, hxk2Δ, and cbp2Δ cells upon glucose starvation for the indicated time points before imaging. n=312, 186, 209,194, 209, 186 cables from ~20 cells per condition. (C, D) Representative images of the ATP-sensor QUEEN in the indicated yeast by excitation at 410 nm or 480 nm and ratiometric analysis (from left to right: n=115, 73, 96, 94, 128, 99 cells). (E, F) Representative images of Abp140-3GFP in WT, spa2Δ, and spa2Δcbp2Δ grown upon rapid GS for 5 min and fluorescence intensity quantification. n=195, 244 and 198 cables from >20 cells per condition. (G) The normalized QUEEN ratio (410 nm ex/480 nm ex) in WT, spa2Δ, and spa2Δcbp2Δ cells was calculated from the signal intensity of each pixel to generate the QUEEN ratio image of cells. n=82, 84 and 56 cells from left to right. (H) Dual-colour images of Spa2-GFP and Abp140-Tomato upon ES under the indicated conditions. (I) Co-localization analysis of Spa2-GFP and Abp140-Tomato by 20-pixel-long line cross filaments. (J, K) Representative maximum Z-projection images of Spa2-GFP localization in WT, hxk2Δ and cbp2Δ after 5 min and 30 min of GS and quantification of the pattern distribution of Spa2 under the indicated conditions. n=150, 175, 150, 150, 150, 150, 150, 142, 153 cells from left to right. Scale bars, 2 μm.

FIG. 2 depicts the results showing that Spa2-535 regulates actin cable remodelling upon ES. (A) Intrinsically disordered region analysis. (B) Representative fluorescent images of GFP-tagged Spa2 truncation variants upon ES under the indicated conditions. (C, D) Quantification of the mean signal intensity of Spa2-535-GFP filaments and puncta of Spa2-281-535-GFP upon ES (n=14, 19, 20 and 20 cells for each condition). (E) Localization of Abp 140-3GFP in Spa2 truncating mutants, with or without ES at 5 min. (F, G) Quantification of the mean intensity of Abp140-3GFP per filament and total signal intensity per indicated Spa2 truncating mutant cells in the presence or absence of ES (n>90 cables per treatment). Scale bars, 2 μm.

FIG. 3 depicts the results indicating that Spa2-535 nucleates and elongates ADP-actin. (A) Fluorescence anisotropy profile of the indicated recombinant Spa2-N-terminal truncation variants (60 nM, Alexa488-labeled) that were titrated with increasing concentrations of ADP-G-actin. Measurements from three biological replicates were plotted and fit with the Hill equation. (B) Representative time-lapse TIRFM images of actin depolymerization by incubating 50 nM Spa2-truncation variants and 50 nM Cof1 with preformed F-actin using 0.5 UM ATP-G-actin with 10% Oregon Green 488 (OG488) and 0.5% biotin labeling. (C) Quantification of total F-actin signal intensity at the indicated time points. (D) Representative time-lapse TIRFM images of actin polymerization at the indicated time point using 3 μM ADP-actin (10% OG488- and 0.5% biotin-labeled), with or without the indicated 10 nM Spa2 variants. (E) Comparison of nucleation efficiency at 5 min by measuring the seed number (from left to right: n=22, 28, 29, and 29 ROIs at 64×64 μm²). (F) Quantification of the actin elongation rates and kymograph in (E) (left to right, n=33, 35, 38, and 51 actin filaments). Bars for B and D, 5 μm; Bar for F, 2 μm. Error bars, mean±S.D.

FIG. 4 shows the results indicating that Spa2 promotes ADP-F-actin bundling. (A-C) Sedimentation velocity profiles of Spa2-281-535 (18 μM), Spa2-535 (13 UM), and Spa2-281 (17 μM), containing the calculated apparent molecular weights (MWₐₚₚ). The sedimentation coefficient and fiction ratio (f/f0) of the major species of each Spa2 variant are indicated. The predicted oligomerization status was indicated based on differential sedimentation coefficient distribution analysis. (D-F) SPR sensorgrams of self-interacting Spa2-281-535, Spa2-535, and Spa2-281. The corresponding curves were fitted in the bivalent model. (G) Phase separation of 5 UM Spa2-535, Spa2-281-535 and Spa2-281 (10% Alexa 488-labeled) at 50 mM KCl. (H) Spa2-535 phase diagram showing protein concentration and KCl concentration dependence. (I) Transmission light time-lapse images of representative coalescence of 5 μM Spa2-535 at 50 mM KCl. (J) Fluorescent recovery analysis of Spa2-535 proteins after photobleaching. Scale bar, 5 μm.

FIG. 5 depicts the multifaceted remodelling of ADP-actin by Spa2 in a D-loop conformation-dependent manner. (A) Fluorescence micrographs of ADP-F-actin incubated with 5 μM Alexa 488-labeled Spa2-281, Spa2-535, and Spa2-281-535 and labeled with Alexa 565-phalloidin before being imaged by spinning disk confocal microscope-coupled super-resolution imaging. (B) Representative time-lapse TIRFM images of ADP-actin formed at the indicated time point in the presence of 3 μM ADP-actin (10% OG488- and 0.5% biotin-labeled) and 5 UM Spa2 variants. Spa2-281-535-trimer-bundled ADP-F-actin was fixed and stained with Alexa565-phalloidin (C) or imaged over time via TIRFM (D) under the same conditions as in (B). (E) Fluorescence micrographs of ADP-actin filaments that were pre-incubated with phalloidin or jasplakinolide for 30 min at a 1:1 molar ratio before adding 10 UM Spa2-535. Fluorescence micrographs (F) and fluorescent signal quantification (G, H) of Alexa565-phalloidin-stained actin cables in WT, act1-159, act1-125, and act1-101 after 5 min of 2-DG treatment. n=127, 66, 183 and 222 cables per treatment for (G) and n=20, 12, 28 and 33 cells (left to right) for (H). Scale bars are 2 μm in A, C, F, and 5 μm in B, D, E.

FIG. 6 shows energy starvation-induced actin cable remodelling in yeast. (A) Quantification of the total fluorescence intensity of the ABP140-3GFP-labeled actin cables (n=30, 22, 25, 19, 22, 18 cells, left to right). (B) Representative maximum Z-projection images of ABP140-3GFP-labeled actin cables in WT and glucose-relevant mutants hxk2Δ and cbp2Δ treated with 20 mM 2-DG for 5 min. (C) Quantification of the mean and total fluorescence intensity of the ABP140-3GFP-labeled actin cables in (B) (from left to right: n=226, 132 and 192 cables; n=19, 15 and 20 cells). (D) The normalized QUEEN ratio (410 nm ex/480 nm ex) in WT, cbp2Δ, and hxk2Δ upon 5 min of 2-DG treatment. The ratio was calculated from the signal intensity of each pixel to generate the QUEEN ratio image of cells (from left to right: n=103, 103 and 80 cells). (E) Representative maximum Z-projection images of ABP140-3GFP in WT, gpr1Δ, and snf3Δrgt2Δ cells upon ES for 5 min and quantification of the mean (F) (n=211, 247, 277 and 224 cables, left to right) or total fluorescence intensity (G) (n=21, 23, 25,19 cells, left to right). (H) The normalized QUEEN ratio (410 nm ex/480 nm ex) in WT, gpr1Δ, and snf3Δrgt2Δ cells was calculated after 5 min of ES using the signal intensity of each pixel to generate the QUEEN ratio image of cells (from left to right: n=105, 105, 97 and 92 cells). Representative maximum Z-projection images of Spa2-GFP localization in wild-type and glucose-relevant yeast mutants hxk2Δ, cbp2Δ, gpr1Δ, snf3Δrgt2Δ grown in SM medium and transferred to glucose starvation and energy depletion for 5 min. Scale bars, 2 μm.

FIG. 7 depicts the screening of ES-triggered actin remodelling in yeast mutants. (A) Representative maximum Z-projection images of ABP140-3GFP-labeled actin cables in WT actin-binding protein mutants upon 5 min ES. (B) Quantification of the mean and total fluorescence intensity of the ABP140-3GFP-labeled actin cables in (A). (C) Quantification of the total fluorescence intensity of the ABP140-3GFP-labeled actin cables in WT, spa2Δ, and spa2Δcbp2Δ cells upon 5 min of glucose starvation (n=22, 26 and 23 cells, left to right). (D) Representative maximum Z-projection images of Spa2-GFP localization in wild-type, hxk2Δ, cbp2Δ, after 5 min GS, (E) actin-binding protein mutants, or (F) glucose sensing mutants gpr1Δ and snf3Δrgt2Δ. Scale bar, 2 μm.

FIG. 8 shows Spa2-mediated actin remodelling upon ES is through the N-terminus. (A) Domain illustration of the Spa2 truncating variants used in the study. Localization and pattern changes of GFP-tagged Spa2 truncating variants upon 5 min ES conditions. (B) Representative fluorescence imaging of GFP-tagged Spa2-535 in the indicated mutants upon glucose starvation for 5 min. (C, D) Quantification of the mean or total signal intensity of Spa2-535-GFP filaments in (B) (from left to right: n=152, 242 and 256 per filament in C; n=22, 26 and 23 cells in D). (E) Representative maximum Z-projection imaging of Alexa 565-phalloidin-stained yeasts expressing Spa2-GFP, Spa2-1306-GFP, Spa2-1087-GFP, Spa2-816-GFP, Spa2-535-GFP, Spa2-281-535-GFP, and Spa2-281-GFP after 5 min of 2-DG treatment. Scale bar, 2 μm.

FIG. 9 depicts nucleotide-specific actin polymerization by Spa2. (A) SDS-PAGE of bacteria purified recombinant Spa2-281, Spa2-535, and Spa2-281-535. (B) Anisotropic measurements of 60 nM Alexa488-labeled Spa2-N titrated with increasing concentrations of ATP-G-actin. The average values with an error bar of +SD were calculated from three biological replicates and plotted with the Hill slope equation. (C) Analysis of severing activities by measuring the cumulative severing events per micron of F-actin at the indicated time point. Left panel: Averaged values±SD from three independent experiments are shown and fit to an exponential association curve. Right panel: Average time+SD to half-maximal severing was calculated from exponential curve fits of the data in (C). (D) Representative TIRF-actin polymerization time-lapse images at the indicated time points. Actin filaments were assembled using 0.5 UM ATP-actin (10% OG488 and 0.5% biotin-labeled) with or without 10 nM Spa2-535, Spa2-281-535, or Spa2-281. The scale bar represents 5 μm. (E) Quantification of the number of nucleated actin seeds at 5 min (from left to right: n=29, 28, 29, and 29 ROIs at a size of 64×64 μm²). The box plot covers data from minimal to maximal, with the central line indicating the mean value. (F) Quantification of the actin elongation rates and kymograph in (D) (from left to right: n=71, 58, 24, and 41 filaments). Data correspond to the mean±S.D. The scale bar represents 2 μm. (G) Representative TIRFM images of actin filaments formed at the indicated time point in the same field. Actin filaments were assembled using 3 μM ADP-Pi-actin (10% Oregon green 488 labeled and 0.5% biotin-actin) with or without 10 nM Spa2-535 and 10 nM Spa2-281-535. The scale bar represents 5 μm. (H) Quantification of the number of nucleated actin seeds at 5 min (left to right, n=63, 63, and 63 ROIs at a size of 19×19 μm²). The box plot covers data from minimal to maximal, with the central line indicating the mean value. (I) Quantification of the actin elongation rates (mean±S.D.) and kymograph was derived from TIRFM movies as shown in (G) in the presence of different Spa2 proteins at the concentration indicated. (left to right, n=48, 49, and 52 actin filaments). Scale bar, 4 μm.

FIG. 10 shows that F-actin is bundled by Spa2 in a nucleotide-specific manner. (A) Representative time-lapse images of Spa2-535 droplets in the FRAP assay in buffer with 50 mM KCl. (B) Fluorescence micrographs of 3 μM ADP-F-actin filaments or 0.5 μM ATP-F-actin filaments that were incubated with a series of concentrations of Spa2-535 at 0, 0.25, 0.5, 1, 2.5, 5, and 10 UM prior to Alexa565-phalloidin staining and imaging. (C) Transmission light microscopic imaging of Spa2-535 phase separation at the indicated concentrations that were used in (B). (D) Fluorescence micrographs of actin filaments incubated with or without 5 μM Alexa488-labeled Spa2-281, Spa2-535, and Spa2-281-535 before being subjected to Alexa565-phalloidin staining and imaging. (E) Representative time-lapse TIRFM images of ATP-actin polymerization with the indicated recombinant Spa2 variants. Actin filaments were assembled using 0.5 μM ATP-actin (10% OG488- and 0.5% biotin-labeled) with or without 5 UM Spa2-281, Spa2-535, or Spa2-281-535. Scale bars are 5 μm in B, C, E, and 2 μm in A, D.

FIG. 11 depicts engineered Spa2-281-535 oligomers for ADP-actin polymerization and functional characterization of D-loop for Spa2-specific interaction. (A) Cartoon depicting protein engineering of Spa2-281-535 using homotrimeric CC motifs. (B) Comparison of Spa2-281-535 and Spa2-281-535-trimer on SDS-PAGE. (C) Sedimentation velocity analysis of the Spa2-281-535 trimer. (D) Representative time-lapse TIRFM images of actin polymerization. F-actin was polymerized using 3 μM ADP-actin or 0.5 μM ATP-actin (10% OG488- and 0.5% biotin-labeled), with or without 10 nM Spa2-281-535 or Spa2-281-535-trimer. (E, F) Quantification of nucleated ADP-actin seeds (from left to right: n=69, 71, 71 ROIs sized at 13.2×13.2 μm²), ADP-actin elongation rates (from left to right: n=84, 121, 123 actin filaments) and kymography. The box plot shows the mean±S.D. (G, H) Quantification of ATP-actin seed generation at 8 min (from left to right: n=28, 29, 30 ROIs at a size of 13.2×13.2 μm²), elongation rates (from left to right: n=32, 29, 39 actin filaments) and kymography in the presence of 10 nM Spa2 variants. (I) Fluorescence micrographs of actin filaments incubated with or without 5 UM Alexa488-labeled Spa2-281-535-trimer prior to Alexa565-phalloidin staining and imaging. (J) Representative time-lapse TIRFM images of ATP-actin bundling, with or without 5 UM Spa2-281-535-trimer, using 0.5 μM ATP-actin (10% OG488- and 0.5% biotin-labeled). (K) Structural information of ADP-bound G-actin (PDB code: 2hf3) with highlighted mutant sites in actin mutants act1-159, act1-125, and act1-101. Illustration of actin binding positions of phalloidin (PDB: 7bti) and jasplakinolide (PDB: 7ply). Scale bars for D and J, 5 μm. Scale bars are 2 μm in F, H and I.

FIG. 12 depicts the rational design of an ADP-actin specific binding motif based on structural predictions (A, B) Alphafold's predicted structure for the ADP-actin binding region Spa2-389-535 and its sequences. (C, D) Detailed analysis of identified Helix 1, responsible for ADP-actin specific binding, comprising a short and a long helix surrounded by flexible loops. New constructs expressing msfGFP fusions were designed for individual helices, labeled H1a and H1b.

FIG. 13 depicts the identification and characterization of an ADP-actin sensing peptide through in vitro biochemistry and in vivo cellular imaging using msfGFP-Spa2-H1b reporter. (A) Domain diagram of Spa2 truncating variants and SDS-PAGE of bacterially purified recombinant Spa2-H1-H4 (residues 389-535), Spa2-H1 (residues 389-433), Spa2-H1a (residues 389-407), and Spa2-H1b (residues 401-433). (B) Fluorescence micrographs of ADP-actin filaments incubated with 5 UM Spa2-H1-H4-GFP, Spa2-H1-GFP, Spa2-H1a-GFP, Spa2-H1b-GFP, and labelled with 565-phalloidin before imaging with a spinning disk confocal microscope-coupled super-resolution system, scale bar 2 μm. (C) Fluorescence micrographs of ATP-actin filaments incubated with 5 UM Spa2-H1-H4-GFP, Spa2-H1-GFP, Spa2-H1a-GFP, Spa2-H1b-GFP, and labelled with 565-phalloidin before imaging with a spinning disk confocal microscope-coupled super-resolution system, scale bar 2 μm. (D) Representative U2OS cells expressing Spa2-H1-GFP, exposed to DMEM with 0.45% glucose or DMEM with 0.45% 2-DG but no glucose for 10 min, then fixed and stained with phalloidin 565, scale bar 5 μm. (E) Representative U2OS cells expressing Spa2-H1b-GFP, exposed to DMEM with 0.45% glucose or DMEM with 0.45% 2-DG but no glucose for 10 min, then fixed and stained with phalloidin 565, scale bar 10 μm.

FIG. 14 shows the identification and characterization of the minimal helix required for ADP-actin sensing in vitro and in vivo. (A) Domain diagram of Spa2 truncating variants. (B) Predicted structure of Saccharomyces cerevisiae's Spa2 corresponding to residues H1b, H1b1, H1b2, and H1b3. (C) SDS-PAGE of bacterially purified recombinant Spa2-H1b, Spa2-H1b1, Spa2-H1b2, and Spa2-H1b3. (D, E) Fluorescence micrographs of ATP-actin and ADP-actin filaments incubated with 5 UM Spa2-H1b1-GFP, Spa2-H1b2-GFP, Spa2-H1b3-GFP, and labeled with 565-phalloidin before imaging with a spinning disk confocal microscope-coupled super-resolution system, scale bar 2 μm. (F, G, H) Representative U2OS cells expressing Spa2-H1b1-GFP, Spa2-H1b2-GFP, and Spa2-H1b3-GFP, exposed to DMEM with 0.45% glucose or DMEM with 0.45% 2-DG but no glucose for 10 min, then fixed and stained with phalloidin 565, scale bar 10 μm.

### DETAILED DESCRIPTION OF THE INVENTION

The following detailed description refers to, by way of illustration, specific details and embodiments in which the invention may be practiced. These embodiments are described in sufficient detail to enable those skilled in the art to practice the invention. Other embodiments may be utilized and structural, and logical changes may be made without departing from the scope of the invention. The various embodiments are not necessarily mutually exclusive, as some embodiments can be combined with one or more other embodiments to form new embodiments.

Bibliographic references mentioned in the present specification are for convenience listed in the form of a list of references and added at the end of the examples. The whole content of such bibliographic references is herein incorporated by reference but their mention in the specification does not imply that they form part of the common general knowledge.

### Definitions

For convenience, certain terms employed in the specification, examples and appended claims are collected here.

In general, technical, scientific and medical terminologies used herein has the same meaning as understood by those skilled in the art to which this invention belongs. Further, the following technical comments and definitions are provided. These definitions should in no way limit the scope of the present invention to those terms alone, but are put forth for a better understanding of the following description.

As used herein, “a” or “an” may mean one or more than one unless indicated to the contrary or otherwise evident from the context.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

As used herein, the term “energy starvation” or “energy starvation condition” may refer to a condition in which the energy status or level in a cell is lower than what is required for the cell to perform its function (e.g., maintaining the equilibrium between catabolism and anabolism). In this regard, the cell may be deprived of one or more nutrients, e.g., glucose etc., which may lead to a loss of ATP, which is the cellular energy currency. Accordingly, “energy starvation condition” may also be interpreted to comprise any type of stress that would lead to a loss of ATP in the cell. It would be appreciated by a skilled person that cellular energy levels may be monitored by measuring ATP/ADP ratio in a cell.

As used herein, the term “functional fragment” refers to a portion of a polypeptide/protein that retains some or all of the activity or function (e.g., biological activity or function, such as enzymatic activity) of the reference polypeptide/protein, such as, e.g., the ability to bind and/or interact with or modulate another protein or polynucleic acid. As used in the context of the present application, the functional fragment of the polypeptide of the present invention may have the ability to bind to ADP-actin and modulate ADP-actin remodelling. The functional fragment can be any size, provided that the fragment retains the activity/functionality of the reference polypeptide/protein.

As used herein, the terms “peptide”, “polypeptide” and “protein” are used interchangeably to denote a polymer of at least two amino acids covalently linked by an amide bond. Whereas peptides are considered to be short amino acid chains, polypeptides are long amino acid chains and proteins tend to have a stable structure and may comprise modifications (e.g., glycosylation or phosphorylation). The term “polypeptide” or “protein” may encompass a naturally-occurring as well as artificial (e.g., engineered or variant) full-length polypeptide/protein as well as a functional fragment of the polypeptide/protein.

As used herein, the term sequence “variant”, refers to an amino acid sequence that is altered by one or more amino acids of the non-variant reference sequence, but retains the ability to recognize its target and effect its function. For example, a polypeptide variant of the present invention is altered by one or more amino acids of the non-variant polypeptide reference sequence, but retains the ability to bind to ADP-actin and modulate ADP-actin remodelling. ADP-actin remodelling may include, for example, enhancing ADP-actin nucleation, elongation/polymerization, crosslinking, bundling, stabilization and/or inhibition of actin depolymerization. The variant may have “conservative” changes, wherein a substituted amino acid has similar structural or chemical properties (e.g., replacement of leucine with isoleucine). More rarely, a variant may have “non-conservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, DNASTAR® software (DNASTAR, Inc. Madison, Wisconsin, USA). A homologous sequence from another species is considered to be a variant of the polypeptide of the invention.

The terms “nucleotide” and “nucleic acid” refer to naturally occurring ribonucleotide or deoxyribonucleotide monomers, as well as non-naturally occurring derivatives and analogs thereof. Nucleotides can include, for example, nucleotides comprising naturally occurring bases (e.g., adenosine, thymidine, guanosine, cytidine, uridine, inosine, deoxyadenosine, deoxythymidine, deoxyguanosine, or deoxycytidine) and nucleotides comprising modified bases known in the art. Accordingly, the term “polynucleotide” and “polynucleic acid” relate in general to polyribonucleotides and polydeoxyribonucleotides, it being possible for these to be non-modified RNA or DNA or modified RNA or DNA.

The term “recombinant” as used herein, means that a molecule (e.g., a polynucleic acid or a polypeptide) has been artificially or synthetically (i.e., non-naturally) altered by human intervention. The alteration can be performed on the molecule within, or removed from, its natural environment or state.

As used herein, the term “subject” is herein defined as a vertebrate, particularly a mammal, more particularly a human. For purposes of research, the subject may particularly be at least one animal model, e.g., a mouse, rat and the like. In other examples, for treatment or prophylaxis of a disease associated with actin remodelling, the subject may be a human.

The term “treatment”, as used in the context of the invention refers to ameliorating, therapeutic or curative treatment.

A description of exemplary, non-limiting embodiments of the invention follows.

The present invention is based, in part, on the development of a recombinant polypeptide capable of regulating actin remodelling. The recombinant polypeptides disclosed herein are derived from the SPA2 gene of Saccharomyces cerevisiae and are highly selective for ADP-actin (as opposed to the ATP state of actin, ATP-actin for example). In particular, the recombinant polypeptides disclosed herein are able to modulate ADP-actin remodelling induced under energy starvation conditions. As ADP-actin is a crucial component of the actin cytoskeleton network, selective targeting of ADP-actin may provide novel pathways for the development of actin cytoskeleton biosensors, the development of drugs and therapies for actin-associated diseases as well as conditions related to energy depletion-related cellular processes, including aging and stress adaptation.

To this end, provided in one aspect of the present disclosure is a recombinant polypeptide comprising an amino acid sequence of at least 85%, at least 90%, at least 95%, at least 96%, at least 97%, at least 98%, or at least 99% identity to the amino acid sequence set forth in SEQ ID NO: 1, or SEQ ID NO: 2, or a functional fragment or functional variant thereof, wherein the polypeptide, fragment or variant thereof specifically binds to ADP-actin and modulates ADP-actin remodelling.

TABLE 1
Amino acid and DNA sequences of the recombinant
polypeptides of the present invention

| | | | | |
|---|---|---|---|---|
| SEQ ID NO: 1 | MGTSSEVSLA | HHRDIFHYYV | SLKTFFEVTG | ENRDRSNSTR |
| Spa2 | AQKARAKLLK | LSSSOFYELS | TDVSDELQRR | IGEDANQPDY |
| (Saccharomyces | LLPKANFHMK | RNQARQKLAN | LSQTRENDLL | DDILFEIKRR |
| cerevisiae) | GFDKDLDAPR | PPLPQPMKQE | VSKDSDDTAR | TSTNSSSVTQ |
| Amino acid | VAPNVSVQPS | LVIPKMASID | WSSEEEEEEQ | VKEKPNEPEG |
| sequence | KQTSMDEKKE | AKPALNPIVT | DSDLPDSQVL | ARDITSMART |
| (Length: 1466 | PTTTHKNYWD | VNDSPIIKVD | KDIDNEKGPE | QLKSPEVQRA |
| amino acids) | ENNNPNSEME | DKVKELTDLN | SDLHLQIEDL | NAKLASLTSE |
| | KEKEKKEEKE | EKEKEKNLKI | NYTIDESFOK | ELLSLNSQIG |
| | ELSIENENLK | QKISEFELHQ | KKNDNHNDLK | ITDGFISKYS |
| | SADGLIPAQY | ILNANNLIIQ | FTTRLSAVPI | GDSTAISHQI |
| | GEELFOILSQ | LSNLISOLLL | SADLLOYKDQ | VILLKASLSH |
| | AITSIRYFSV | YGPVLIPKIT | VOAAVSEVCF | AMCNLIDSAK |
| | IKSDSNGEST | TSNEGNRQVL | EYSSPTATTP | MTPTFPSTSG |
| | INMKKGFINP | RKPASFLNDV | EEEESPVKPL | KITQKAINSP |
| | IIRPSSSNGV | PTTSRKPSGT | GLFSLMIDSS | IAKNSSHKED |
| | NDKYVSPIKA | VTSASNSASS | NISEIPKLTL | PPQAKIGTVI |
| | PPSENQVPNI | KIENTEEDNK | RSDITNEISV | KPTSSIADKL |
| | KOFEQSSEKK | SSPKENPIAK | EEMDSKPKLS | NKFITSMNDV |
| | STDDSSSDGN | ENDDADDDDD | FTYMALKOTM | KREGSKIEKN |
| | NDSKLPANIV | ELDLHESPES | VKIESPESIK | EITSSEMSSE |
| | MPSSSLPKRL | VEDVEPSEMP | EKGASVESVR | KKNFQEPLGN |
| | VESPDMTQKV | KSLGMTGKAV | GPESDSRVES | PGMTGQIKSL |
| | NMAGKVVGPE | ADSRVESPGM | KEQIKSLGMT | GKITAQESIK |
| | SPEAARKLAS | SGEVDKIESP | RMVRESESLE | AVGNTIPSNM |
| | TVKMESPNLK | GNTVSEPQEI | RRDIASSEPI | ENVDPPKVLK |
| | KIVFPKAVNR | TGSPKSVEKT | PSSATLKKSG | LPEPNSQIVS |
| | PELAKNSPLA | PIKKNVELRE | TNKPHTETIT | SVEPTNKDAN |
| | TSWRDADLNR | TIKREEEDED | FDRVNHNIQI | TGAYTKTGKI |
| | DYHKIPVDRK | AKSEAEVHTS | EEDIDESNNV | NGKRADAQIH |
| | ITERKHAFVN | PTENSQVKKT | SHSPFLNSKP | VOYENSESNG |
| | GINNHIKIKN | TGETTAHDEK | HYSDDDDSSY | QFVPMKHEEQ |
| | EQEQNRSEEE | ESEDDDEEEE | DSDEDVDTED | IENPDNTLSE |
| | LLLYLEHQTM | DVISTIQSLL | TSIKKPQVTK | GNLRGESNAI |
| | NQVIGQMVDA | TSISMEQSRN | ANLKKHGDWV | VQSLRDCSRR |
| | MTILCOLTGD | GILAKEKSDQ | DYADKNFKOR | LAGIAFDVAK |
| | CTKELVKTVE | EASLKDEINY | LNSKLK | |
| SEQ ID NO: 2 | MGTSSEVSLA | HHRDIFHYYV | SLKTFFEVTG | ENRDRSNSTR |
| Spa2-535 | AQKARAKLLK | LSSSQFYELS | TDVSDELORR | IGEDANQPDY |
| Amino acid | LLPKANFHMK | RNOARQKLAN | LSQTRENDLL | DDILFEIKRR |
| sequence | GFDKDLDAPR | PPLPQPMKQE | VSKDSDDTAR | TSTNSSSVTQ |
| (Length: 535 | VAPNVSVOPS | LVIPKMASID | WSSEEEEEEQ | VKEKPNEPEG |
| amino acids) | KQTSMDEKKE | AKPALNPIVT | DSDLPDSQVL | ARDITSMART |
| | PTTTHKNYWD | VNDSPIIKVD | KDIDNEKGPE | QLKSPEVQRA |
| | ENNNPNSEME | DKVKELTDLN | SDLHLQIEDL | NAKLASLTSE |
| | KEKEKKEEKE | EKEKEKNLKI | NYTIDESFOK | ELLSLNSQIG |
| | ELSIENENLK | QKISEFELHQ | KKNDNHNDLK | ITDGFISKYS |
| | SADGLIPAQY | ILNANNLIIQ | FTTRLSAVPI | GDSTAISHQI |
| | GEELFOILSQ | LSNLISQLLL | SADLLOYKDQ | VILLKASLSH |
| | AITSIRYFSV | YGPVLIPKIT | VOAAVSEVCF | AMCNLIDSAK |
| | IKSDSNGEST | TSNEG | | |
| SEQ ID NO: 3 | NNNPNSEME | DKVKELTDLN | SDLHLQIEDL | NAKLASLTSE |
| Spa2-281-535 | KEKEKKEEKE | EKEKEKNLKI | NYTIDESFOK | ELLSLNSQIG |
| Amino acid | ELSIENENLK | QKISEFELHQ | KKNDNHNDLK | ITDGFISKYS |
| sequence | SADGLIPAQY | ILNANNLIIQ | FTTRLSAVPI | GDSTAISHQI |
| (Length: 254 | GEELFOILSQ | LSNLISQLLL | SADLLQYKDQ | VILLKASLSH |
| amino acids) | AITSIRYFSV | YGPVLIPKIT | VOAAVSEVCF | AMCNLIDSAK |
| | IKSDSNGEST | TSNEG | | |
| SEQ ID NO: 4 | LK | ITDGFISKYS | SADGLIPAQY | ILNANNLIIQ |
| Spa2-H1-H4 | FTTRLSAVPI | GDSTAISHQI | GEELFOILSQ | LSNLISQLLL |
| (residues 389- | SADLLQYKDQ | VILLKASLSH | AITSIRYFSV | YGPVLIPKIT |
| 535) | VQAAVSEVCF | AMCNLIDSAK | IKSDSNGEST | TSNEG |
| Amino acid | | | | |
| sequence | | | | |
| (Length: 147 | | | | |
| amino acids) | | | | |
| SEQ ID NO: 5 | LK | ITDGFISKYS | SADGLIPAQY | ILNANNLIIQ |
| Spa2-H1 | FTTRLSAVPI | GDS | | |
| (residues 389- | | | | |
| 433) | | | | |
| Amino acid | | | | |
| sequence | | | | |
| (Length: 45 | | | | |
| amino acids) | | | | |
| SEQ ID NO: 6 | QY | ILNANNLIIQ | FTTRLSAV | |
| Spa2-H1b3 | | | | |
| (residues 409- | | | | |
| 428) | | | | |
| Amino acid | | | | |
| sequence | | | | |
| (Length: 20 | | | | |
| amino acids) | | | | |
| SEQ ID NO: 7 | MGTSSEVSLA | HHRDIFHYYV | SLKTFFEVTG | ENRDRSNSTR |
| Intrinsically | AQKARAKLLK | LSSSQFYELS | TDVSDELQRR | IGEDANQPDY |
| disordered | LLPKANFHMK | RNQARQKLAN | LSQTRENDLL | DDILFEIKRR |
| region (IDR, aa | GFDKDLDAPR | PPLPQPMKQE | VSKDSDDTAR | TSINSSSVTQ |
| 1-281) | VAPNVSVQPS | LVIPKMASID | WSSEEEEEEQ | VKEKPNEPEG |
| Amino acid | KQTSMDEKKE | AKPALNPIVT | DSDLPDSQVL | ARDITSMART |
| sequence | PTTTHKNYWD | VNDSPIIKVD | KDIDNEKGPE | QLKSPEVQRA |
| (Length: 281 | E | | | |
| amino acids) | | | | |

| | |
|---|---|
| SEQ ID NO:8 | ATGGGTACGTCAAGCGAGGTTTCTCTCGCACATCATAGAGATATC |
| Spa2 full | TTCCATTACTACGTCTCACTGAAGACTTTTTTCGAGGTGACTGGC |
| length | GAAAATCGTGACAGGTCAAATTCGACACGAGCTCAAAAGGCCAGA |
| DNA sequence | GCCAAGCTGTTGAAGCTATCTTCTTCGCAATTTTACGAGCTGAGT |
| | ACAGACGTGTCCGATGAGCTGCAGAGGAGAATCGGTGAAGATGCT |
| | AACCAACCAGATTACCTTTTGCCGAAGGCAAATTTCCACATGAAA |
| | AGGAACCAGGCTAGACAGAAACTGGCCAATCTATCACAAACTCGA |
| | TTTAATGATTTGTTGGACGATATCCTTTTTGAGATCAAGAGAAGA |
| | GGGTTCGACAAGGATTTGGATGCTCCACGGCCCCCATTACCGCAG |
| | CCGATGAAACAAGAGGTCAGCAAAGACAGCGATGATACTGCAAGA |
| | ACATCCACAAATTCTTCCTCTGTGACTCAAGTAGCTCCAAACGTC |
| | TCCGTACAACCTTCTTTGGTCATTCCTAAGATGGCATCTATCGAT |
| | TGGTCTTCTGAGGAAGAAGAAGAGGAGCAAGTAAAGGAGAAGCCA |
| | AATGAACCGGAGGGAAAACAAACAAGCATGGATGAAAAGAAAGAG |
| | GCTAAACCTGCTCTAAACCCCATAGTTACAGATTCTGATCTGCCT |
| | GACTCCCAAGTGCTCGCTCGTGATATCACATCAATGGCAAGGACT |
| | CCAACAACGACGCATAAAAATTACTGGGACGTTAATGATTCTCCA |
| | ATTATCAAGGTAGATAAAGATATCGATAACGAAAAGGGTCCCGAA |
| | CAGTTGAAGAGCCCTGAAGTACAACGGGCTGAGAACAATAACCCT |
| | AACTCAGAGATGGAAGACAAGGTTAAAGAACTGACTGATTTAAAC |
| | AGCGACTTACATTTGCAAATTGAAGATTTGAATGCTAAGTTAGCA |
| | TCTTTAACCAGCGAGAAGGAAAAGGAGAAGAAGGAAGAGAAGGAG |
| | GAAAAAGAAAAGGAAAAGAACTTAAAGATTAACTACACCATTGAT |
| | GAAAGTTTTCAGAAAGAATTGCTGTCATTAAACTCTCAAATCGGT |
| | GAATTATCAATTGAGAATGAAAATTTGAAGCAGAAAATTTCAGAA |
| | TTCGAACTGCATCAAAAAAAGAATGACAACCATAATGATTTGAAA |
| | ATCACTGACGGTTTTATTAGCAAGTACTCTTCTGCCGATGGGCTC |
| | ATTCCAGCTCAATACATCTTAAACGCTAACAACTTGATAATTCAA |
| | TTTACTACTAGGCTTTCCGCAGTACCCATAGGCGACTCCACGGCA |
| | ATTTCCCATCAAATTGGCGAAGAGTTATTTCAAATATTATCCCAG |
| | TTATCGAACCTAATCTCCCAGCTATTACTATCGGCCGACCTATTA |
| | CAGTACAAAGATCAGGTCATTTTACTGAAGGCATCATTATCGCAT |
| | GCGATCACATCGATAAGATATTTCTCTGTTTACGGTCCCGTATTA |
| | ATTCCGAAAATAACTGTGCAAGCTGCTGTTTCAGAGGTTTGTTTT |
| | GCCATGTGTAATCTAATTGATTCAGCGAAAATAAAATCCGATTCA |
| | AATGGTGAGAGCACCACCTCTAATGAAGGTAACCGACAGGTATTA |
| | GAATATTCTTCACCAACTGCTACCACCCCAATGACGCCAACTTTC |
| | CCCTCGACTTCTGGAATAAATATGAAGAAGGGGTTTATAAACCCA |
| | AGAAAACCAGCATCTTTCTTGAATGATGTGGAGGAAGAAGAATCT |
| | CCAGTCAAGCCATTGAAAATTACACAAAAGGCAATTAACAGTCCG |
| | ATCATAAGACCGTCATCGTCTAATGGAGTTCCAACAACCTCAAGA |
| | AAACCTTCAGGAACGGGGCTATTTAGTTTAATGATTGATTCATCA |
| | ATTGCTAAGAATAGCTCCCATAAAGAGGATAATGATAAATATGTC |
| | TCGCCCATAAAGGCAGTAACATCGGCCTCCAATTCTGCAAGTAGC |
| | AATATTTCCGAAATTCCTAAACTAACACTACCTCCACAAGCCAAA |
| | ATCGGTACTGTTATTCCACCGTCAGAGAATCAAGTTCCCAATATT |
| | AAAATCGAGAATACAGAAGAGGATAATAAAAGGAGTGACATAACA |
| | AATGAAATCTCTGTTAAACCAACTTCTAGCATTGCTGATAAACTG |
| | AAACAATTTGAGCAAAGTTCCGAAAAGAAATCATCACCAAAGGAA |
| | AATCCTATAGCAAAAGAAGAAATGGATTCAAAACCAAAACTATCC |
| | AATAAATTTATCACTTCAATGAATGATGTGTCCACAGATGATTCA |
| | AGCTCTGATGGTAACGAAAATGACGATGCAGACGATGATGATGAT |
| | TTTACCTATATGGCATTGAAACAAACAATGAAGAGAGAAGGTTCA |
| | AAAATTGAAAAAAATAATGACAGCAAACTACCTGCAAATATAGTG |
| | GAACTTGATTTACATGAGTCACCGGAGTCCGTGAAGATTGAATCT |
| | CCTGAATCGATAAAGGAAATCACGTCATCTGAAATGTCTTCAGAA |
| | ATGCCAAGTAGTTCGCTGCCTAAGAGATTAGTAGAGGATGTTGAG |
| | CCTTCAGAAATGCCAGAGAAGGGCGCATCTGTAGAATCAGTCAGG |
| | AAGAAAAATTTTCAAGAACCACTTGGTAATGTCGAATCTCCGGAT |
| | ATGACGCAGAAGGTCAAGTCTTTGGGTATGACAGGAAAGGCTGTA |
| | GGCCCAGAATCAGATAGTAGGGTCGAATCTCCGGGCATGACAGGA |
| | CAGATTAAATCTTTGAATATGGCAGGAAAAGTTGTAGGCCCAGAA |
| | GCAGATAGTAGGGTCGAATCTCCGGGCATGAAAGAGCAGATTAAG |
| | TCTTTGGGTATGACAGGAAAAATTACAGCTCAAGAATCAATCAAG |
| | TCCCCGGAAGCGGCTAGGAAGITGGCGTCATCAGGAGAAGTTGAC |
| | AAAATTGAATCTCCAAGAATGGTAAGGGAAAGCGAGTCCTTGGAG |
| | GCAGTAGGCAATACTATCCCCTCAAACATGACAGTGAAAATGGAA |
| | TCCCCAAATTTAAAGGGAAATACTGTGTCTGAACCTCAAGAAATA |
| | AGGAGAGACATTGCCTCCTCAGAGCCGATAGAGAATGTTGACCCC |
| | CCAAAAGTACTAAAAAAGATTGTCTTTCCAAAGGCTGTTAATAGA |
| | ACTGGATCACCAAAATCAGTCGAAAAGACTCCATCTTCAGCTACA |
| | CTGAAAAAGAGCGGGCTCCCAGAACCGAATAGCCAAATTGTTTCT |
| | CCTGAATTGGCAAAAAATTCACCTCTAGCACCGATAAAGAAAAAT |
| | GTCGAGTTACGAGAAACTAATAAACCACATACTGAGACTATCACT |
| | TCTGTGGAACCAACAAACAAGGATGCCAATACTTCTTGGAGAGAC |
| | GCCGACTTAAACCGTACGATCAAACGAGAGGAGGAGGACGAAGAT |
| | TTTGATAGAGTGAACCACAATATCCAGATCACTGGTGCATATACG |
| | AAAACTGGAAAAATTGATTATCATAAAATACCTGTTGATCGTAAA |
| | GCAAAAAGCGAAGCAGAAGTGCATACTTCCGAGGAAGATATTGAT |
| | GAATCAAATAATGTTAATGGAAAAAGAGCTGATGCCCAAATACAC |
| | ATCACTGAAAGAAAGCATGCTTTCGTAAATCCAACTGAAAATTCA |
| | CAGGTAAAAAAGACGAGCCACTCACCATTTTTAAACAGTAAACCG |
| | GTTCAATACGAGAACTCAGAGTCGAACGGCGGCATTAACAACCAC |
| | ATAAAGATAAAAAATACTGGAGAAACTACGGCACATGACGAGAAA |
| | CATTATAGTGATGATGATGATTCTAGCTATCAATTTGTTCCCATG |
| | AAACATGAAGAACAAGAACAAGAACAAAACAGAAGTGAGGAAGAG |
| | GAAAGTGAAGATGACGATGAAGAGGAAGAAGACAGTGATTTTGAT |
| | GTGGATACATTTGACATTGAAAATCCGGATAATACACTATCAGAG |
| | TTACTATTGTATTTAGAACATCAAACAATGGACGTCATATCCACG |
| | ATTCAATCGCTTTTGACATCGATCAAGAAACCACAGGTGACGAAG |
| | GGTAATTTGAGGGGAGAATCGAATGCAATCAACCAAGTCATAGGT |
| | CAAATGGTGGACGCTACTAGCATATCAATGGAGCAAAGCAGAAAT |
| | GCCAATTTGAAGAAACACGGTGATTGGGTGGTGCAAAGTCTAAGA |
| | GACTGTTCGCGTAGAATGACAATTTTGTGCCAATTAACTGGCGAT |
| | GGAATACTAGCGAAGGAAAAGAGCGATCAAGATTATGCTGACAAA |
| | AACTTCAAACAGCGGTTGGCAGGGATTGCGTTTGATGTTGCCAAA |
| | TGTACAAAGGAGCTGGTAAAAACTGTAGAAGAGGCAAGTTTGAAG |
| | GACGAAATAAATTATTTGAATTCGAAGTTGAAGTAA |
| SEQ ID NO:9 | ATGGGTACGTCAAGCGAGGTTTCTCTCGCACATCATAGAGATATC |
| Spa2-535 | TTCCATTACTACGTCTCACTGAAGACTTTTTTCGAGGTGACTGGC |
| DNA sequence | GAAAATCGTGACAGGTCAAATTCGACACGAGCTCAAAAGGCCAGA |
| | GCCAAGCTGTTGAAGCTATCTTCTTCGCAATTTTACGAGCTGAGT |
| | ACAGACGTGTCCGATGAGCTGCAGAGGAGAATCGGTGAAGATGCT |
| | AACCAACCAGATTACCTTTTGCCGAAGGCAAATTTCCACATGAAA |
| | AGGAACCAGGCTAGACAGAAACTGGCCAATCTATCACAAACTCGA |
| | TTTAATGATTTGTTGGACGATATCCTTTTTGAGATCAAGAGAAGA |
| | GGGTTCGACAAGGATTTGGATGCTCCACGGCCCCCATTACCGCAG |
| | CCGATGAAACAAGAGGTCAGCAAAGACAGCGATGATACTGCAAGA |
| | ACATCCACAAATTCTTCCTCTGTGACTCAAGTAGCTCCAAACGTC |
| | TCCGTACAACCTTCTTTGGTCATTCCTAAGATGGCATCTATCGAT |
| | TGGTCTTCTGAGGAAGAAGAAGAGGAGCAAGTAAAGGAGAAGCCA |
| | AATGAACCGGAGGGAAAACAAACAAGCATGGATGAAAAGAAAGAG |
| | GCTAAACCTGCTCTAAACCCCATAGTTACAGATTCTGATCTGCCT |
| | GACTCCCAAGTGCTCGCTCGTGATATCACATCAATGGCAAGGACT |
| | CCAACAACGACGCATAAAAATTACTGGGACGTTAATGATTCTCCA |
| | ATTATCAAGGTAGATAAAGATATCGATAACGAAAAGGGTCCCGAA |
| | CAGTTGAAGAGCCCTGAAGTACAACGGGCTGAGAACAATAACCCT |
| | AACTCAGAGATGGAAGACAAGGTTAAAGAACTGACTGATTTAAAC |
| | AGCGACTTACATTTGCAAATTGAAGATTTGAATGCTAAGTTAGCA |
| | TCTTTAACCAGCGAGAAGGAAAAGGAGAAGAAGGAAGAGAAGGAG |
| | GAAAAAGAAAAGGAAAAGAACTTAAAGATTAACTACACCATTGAT |
| | GAAAGTTTTCAGAAAGAATTGCTGTCATTAAACTCTCAAATCGGT |
| | GAATTATCAATTGAGAATGAAAATTTGAAGCAGAAAATTTCAGAA |
| | TTCGAACTGCATCAAAAAAAGAATGACAACCATAATGATTTGAAA |
| | ATCACTGACGGTTTTATTAGCAAGTACTCTTCTGCCGATGGGCTC |
| | ATTCCAGCTCAATACATCTTAAACGCTAACAACTTGATAATTCAA |
| | TTTACTACTAGGCTTTCCGCAGTACCCATAGGCGACTCCACGGCA |
| | ATTTCCCATCAAATTGGCGAAGAGTTATTTCAAATATTATCCCAG |
| | TTATCGAACCTAATCTCCCAGCTATTACTATCGGCCGACCTATTA |
| | CAGTACAAAGATCAGGTCATTTTACTGAAGGCATCATTATCGCAT |
| | GCGATCACATCGATAAGATATTTCTCTGTTTACGGTCCCGTATTA |
| | ATTCCGAAAATAACTGTGCAAGCTGCTGTTTCAGAGGTTTGTTTT |
| | GCCATGTGTAATCTAATTGATTCAGCGAAAATAAAATCCGATTCA |
| | AATGGTGAGAGCACCACCTCTAATGAAGGT |
| SEQ ID NO:10 | AACAATAACCCTAACTCAGAGATGGAAGACAAGGTTAAAGAACTG |
| Spa2-281-535 | ACTGATTTAAACAGCGACTTACATTTGCAAATTGAAGATTTGAAT |
| DNA sequence | GCTAAGTTAGCATCTTTAACCAGCGAGAAGGAAAAGGAGAAGAAG |
| | GAAGAGAAGGAGGAAAAAGAAAAGGAAAAGAACTTAAAGATTAAC |
| | TACACCATTGATGAAAGTTTTCAGAAAGAATTGCTGTCATTAAAC |
| | TCTCAAATCGGTGAATTATCAATTGAGAATGAAAATTTGAAGCAG |
| | AAAATTTCAGAATTCGAACTGCATCAAAAAAAGAATGACAACCAT |
| | AATGATTTGAAAATCACTGACGGTTTTATTAGCAAGTACTCTTCT |
| | GCCGATGGGCTCATTCCAGCTCAATACATCTTAAACGCTAACAAC |
| | TTGATAATTCAATTTACTACTAGGCTTTCCGCAGTACCCATAGGC |
| | GACTCCACGGCAATTTCCCATCAAATTGGCGAAGAGTTATTTCAA |
| | ATATTATCCCAGTTATCGAACCTAATCTCCCAGCTATTACTATCG |
| | GCCGACCTATTACAGTACAAAGATCAGGTCATTTTACTGAAGGCA |
| | TCATTATCGCATGCGATCACATCGATAAGATATTTCTCTGTTTAC |
| | GGTCCCGTATTAATTCCGAAAATAACTGTGCAAGCTGCTGTTTCA |
| | GAGGTTTGTTTTGCCATGTGTAATCTAATTGATTCAGCGAAAATA |
| | AAATCCGATTCAAATGGTGAGAGCACCACCTCTAATGAAGGT |
| SEQ ID NO:11 | TTGAAAATCACTGACGGTTTTATTAGCAAGTACTCTTCTGCCGAT |
| Spa2-389-535 | GGGCTCATTCCAGCTCAATACATCTTAAACGCTAACAACTTGATA |
| DNA sequence | ATTCAATTTACTACTAGGCTTTCCGCAGTACCCATAGGCGACTCC |
| | ACGGCAATTTCCCATCAAATTGGCGAAGAGTTATTTCAAATATTA |
| | TCCCAGTTATCGAACCTAATCTCCCAGCTATTACTATCGGCCGAC |
| | CTATTACAGTACAAAGATCAGGTCATTTTACTGAAGGCATCATTA |
| | TCGCATGCGATCACATCGATAAGATATTTCTCTGTTTACGGTCCC |
| | GTATTAATTCCGAAAATAACTGTGCAAGCTGCTGTTTCAGAGGTT |
| | GATTCAAATGGTGAGAGCACCACCTCTAATGAAGGT |
| | TGTTTTGCCATGTGTAATCTAATTGATTCAGCGAAAATAAAATCC |
| | GATTCAAATGGTGAGAGCACCACCTCTAATGAAGGT |
| SEQ ID NO:12 | TTGAAAATCACTGACGGTTTTATTAGCAAGTACTCTTCTGCCGAT |
| Spa2-389-433 | GGGCTCATTCCAGCTCAATACATCTTAAACGCTAACAACTTGATA |
| DNA sequence | ATTCAATTTACTACTAGGCTTTCCGCAGTACCCATAGGCGACTCC |
| SEQ ID NO: 13 | CAATACATCTTAAACGCTAACAACTTGATAATTCAATTTACTACT |
| Spa2-H1b3 | AGGCTTTCCGCAGTA |
| (residues 409- | |
| 428) | |
| DNA sequence | |
| SEQ ID NO: 14 | LKITDGFISK YSSADGLIP |
| Spa2-H1a | |
| (residues 389- | |
| 407) | |
| Amino acid | |
| sequence | |
| (Length: 19 | |
| amino acids) | |
| SEQ ID NO: 15 | SADGLIPAQY ILNANNLIIQ FTTRLSAVPI GDS |
| Spa2-H1b | |
| (residues 401- | |
| 433) | |
| Amino acid | |
| sequence | |
| (Length: 33 | |
| amino acids) | |

As would be appreciated by a person skilled in the art, actin remodelling is a coordinated biochemical process which plays an important role in governing the shape and movement of a biological cell. In essence, the actin remodelling process may involve actin monomers elongating into polymers to form actin filaments and disassembling back into monomers in response to signalling cascades from environmental cues. The actin remodelling cycle may comprise the nucleation stage, elongation stage/polymerization, termination stage, capping, crosslinking, bundling, stabilization, cargo motoring, and/or disassembly/depolymerization.

Accordingly in the context of the present invention, the actin remodelling may preferably refer to ADP-actin remodelling, and may comprise enhancing/upregulating ADP-actin nucleation, elongation/polymerization, crosslinking, bundling, stabilization and/or inhibiting actin depolymerization.

In some embodiments, the modulating of ADP-actin remodelling is selected from one or more of the group comprising enhancing ADP-actin nucleation, elongation/polymerization, crosslinking, bundling, stabilization and/or inhibition of actin depolymerization.

In some embodiments, the polypeptides disclosed herein may advantageously function as a nucleation factor to enhance ADP-actin nucleation, i.e., initiating the formation of an ADP-actin nucleus from which an actin filament may elongate. In some embodiments, the polypeptides disclosed herein may function as the nucleation factor of ADP-G-actin.

In some embodiments, the polypeptides disclosed herein may advantageously function as a elongation factor to enhance ADP-actin elongation, i.e., enhancing/upregulating the polymerization of ADP-actin filament at one end to elongate the actin filament. In some embodiments, the polypeptides disclosed herein may function as the elongation factor of ADP-G-actin.

In some embodiments, the polypeptides disclosed herein may enhance ADP-actin nucleation and/or elongation/polymerization.

In some embodiments, the polypeptides disclosed herein may advantageously function as a crosslinking factor to enhance ADP-actin crosslinking and/or bundling, for example, enhancing the assembly of actin filaments into bundles and various architectures of actin networks to drive different critical cellular processes, such as but not limited to cell migration, membrane protrusion, endocytosis, vesicular transport, exocytosis, and the ABP function, e.g. motor activities. In some embodiments, the polypeptides disclosed herein may function as a crosslinking factor of ADP-F-actin.

In some embodiments, the polypeptides disclosed herein may function as a stabilization factor to enhance actin filament stabilization, for example, enhancing the filaments' ability to maintain its structure and/or increasing the filaments' resistance to depolymerization. In some embodiments, the polypeptides disclosed herein may function as a stabilization factor of ADP-F-actin.

In some embodiments, the polypeptides disclosed herein may also advantageously act as a multifunctional ADP-actin modulator.

It has been shown that actin remodelling may be induced under energy starvation conditions. Accordingly in some embodiments, the ADP-actin remodelling may occur under an energy starvation condition. In some embodiments, the energy starvation condition may be glucose starvation.

It would be appreciated by a skilled person that a protein's function is directly related to its structure and sequence, and that there is a positive relationship between sequence identity and function similarity. Accordingly, the sequences of the polypeptides of the present disclosure may be sufficiently varied so long as the peptides maintain their functionality and can exhibit the required activity (for example, the ability to bind to ADP-actin and modulate ADP-actin remodelling). It would also be appreciated that a minimal binding motif would be required for the peptide to bind to its target and exhibit its functionality. In this regard, methods of determining a protein sequence identity are known in the art.

In some embodiments, the polypeptide may comprise an amino acid sequence of at least 85%, at least 90%, at least 95%, at least 96%, at least 97%, at least 98%, or at least 99% identity to the amino acid sequence set forth in SEQ ID NO: 1. In some embodiments, the polypeptide may comprise the amino acid sequence set forth in SEQ ID NO: 1. In some other embodiments, the polypeptide may consist of the amino acid sequence set forth in SEQ ID NO: 1.

In certain embodiments, the polypeptide may comprise an amino acid sequence of at least 85%, at least 90%, at least 95%, at least 96%, at least 97%, at least 98%, or at least 99% identity to the amino acid sequence set forth in SEQ ID NO: 2, which corresponds to amino acids 1-535 of SEQ ID NO: 1. In some embodiments, the polypeptide may comprise the amino acid sequence set forth in SEQ ID NO: 2. In some other embodiments, the polypeptide may consist of the amino acid sequence set forth in SEQ ID NO: 2.

In some embodiments, the polypeptide may comprise or consist of an amino acid sequence of at least 85%, at least 90%, at least 95%, at least 96%, at least 97%, at least 98%, or at least 99% identity to an amino acid sequence selected from the group comprising SEQ ID NO: 3, which corresponds to amino acids 281-535 of SEQ ID NO: 1, SEQ ID NO: 4, which corresponds to amino acids 389-535 of SEQ ID NO: 1, SEQ ID NO: 5, which corresponds to amino acids 389-433 of SEQ ID NO: 1 and SEQ ID NO: 6, which corresponds to amino acids 409-428 of SEQ ID NO: 1.

In certain embodiments, the polypeptide may consist of the amino acid sequence set forth in SEQ ID NO: 3. In certain embodiments, the polypeptide may consist of the amino acid sequence set forth in SEQ ID NO: 4. In certain embodiments, the polypeptide may consist of the amino acid sequence set forth in SEQ ID NO: 5. In certain embodiments, the polypeptide may consist of the amino acid sequence set forth in SEQ ID NO: 6.

In some embodiments, the polypeptide may comprise or consist of the amino acid sequence set forth in SEQ ID NO: 1 or SEQ ID NO: 2 and may enhance ADP-actin crosslinking and/or bundling.

In certain embodiments, the polypeptide may comprise or consist of the amino acid sequence set forth in SEQ ID NO: 3, SEQ ID NO: 4, SEQ ID NO: 5 or SEQ ID NO: 6 and may enhance ADP-actin nucleation and/or elongation/polymerization.

The polypeptides disclosed herein are preferably capable of efficiently binding to actin, particularly to ADP-actin. In some embodiments, the ADP-actin may be ADP-G-actin and/or ADP-F-actin.

The polypeptides of the present disclosure may also be monomeric or multimeric. In some embodiments, the polypeptides may be dimeric or trimeric. In some embodiments, the polypeptides may self-assemble to form multimers. In some embodiments, multimerization of the polypeptides may comprise the use of linkers and/or coiled-coil motif. In some embodiments the multimer is a multimer of polypeptide Spa2-281-535.

In some embodiments, the polypeptide may be coupled to at least one heterologous molecule. For example, the heterologous molecule may be a labelling group which allows for the detection of the polypeptides disclosed herein. In some embodiments, the heterologous molecule may be a labelling peptide such as the FLAG epitope, a labelling fluorescent polypeptide such as GFP, sfGFP, msfGFP or mRFPruby, or any variant thereof. In other embodiments, the heterologous molecule may also be a non-peptidic molecule, e.g. a fluorescent chemical labelling group such as fluorescein, and rhodamine. It would be appreciated that the heterologous molecule may be coupled to the polypeptides of the present disclosure without affecting its binding to ADP-actin and its ability to modulate actin remodelling. For example, a polypeptide of the disclosure may be expressed coupled to a label within a transformed cell to provide the location of the polypeptide in relation to actin undergoing remodelling and, for example, provide an indication of energy status within the cell and/or a relative amount of ADP-actin. In this regard, it is also envisaged that any other heterologous molecules comprising the desired labelling properties may be suitable for use in the practice of the present invention.

In another aspect, there is provided a polynucleic acid molecule encoding the recombinant polypeptide as described herein. For example, the polynucleic acid molecule may be a single- or double-stranded DNA or RNA molecule.

In some embodiments, the polynucleic acid molecule may comprise a sequence of at least 85%, at least 90%, at least 95%, at least 96%, at least 97%, at least 98%, or at least 99% identity to the polynucleic acid sequence set forth in SEQ ID NO: 8 or in SEQ ID NO: 9, or a fragment thereof. In some embodiments, the polynucleic acid sequence comprises or consists of the polynucleic acid sequence set forth in the group comprising SEQ ID NO: 10, SEQ ID NO: 11, SEQ ID NO: 12 and SEQ ID NO: 13.

In some embodiments, the polynucleic acid molecule may be operatively linked to a promoter sequence. Preferably, the promoter sequence is suitably adapted to effectively control the gene expression in a suitable host cell, e.g. a prokaryotic or eukaryotic host cell. In some embodiments, the eukaryotic host cell may be a fungal cell, a plant cell or a mammalian cell.

In another aspect of the disclosure, there is provided a recombinant cell transfected or transformed with a polynucleic acid molecule as described above. In some embodiments, the recombinant cell may be a prokaryotic cell such as an E. coli cell. In other embodiments, the recombinant cell may be a eukaryotic cell such as a fungal cell, a plant cell or an animal cell. Preferably, the animal cell may be a fish, insect, bird or mammalian cell. In this regard, it would be appreciated that standard methods of transfecting or transforming recombinant cells known in the art, such as calcium phosphate precipitation or electroporation, may be applied suitably in the present invention.

In another aspect of the disclosure, there is provided a non-human transgenic organism transfected or transformed with the polynucleic acid molecule as described in the present invention. For example, the transgenic organism may be a fungus, a plant or an animal. In some embodiments, the transgenic organism may be a nematode (e.g., C. elegans), an insect (e.g., Drosophila), a fish (e.g., Danio rero), a mammal (e.g., a rodent) or a plant (e.g., Arabidopsis thaliana). Such organisms provide models for studying ADP-actin remodelling.

In a further aspect of the disclosure, there is provided a use of a recombinant polypeptide, polynucleic acid molecule, or a recombinant cell as described in the present invention in methods for the detection and/or modulation of ADP-actin remodelling, or in drug screening of candidate modulators of ADP-actin remodelling. In view of its selectivity for ADP-actin, the recombinant polypeptides, polynucleic acid molecules and cells disclosed herein may thus be suitably adapted for use as a biosensor for the detection of ADP-actin activity within a cell, preferably within a living cell. For example, the polypeptides of the invention can be tagged with a fluorescence tag and developed to identify ADP-actin forms. A cell may be transfected with a polynucleic acid expression construct for production of GFP-tagged recombinant polypeptide of the invention. To test whether labelled polypeptide selectively associates with subsets of actin structures in a transfected cell, total internal reflection (TIRF) microscopy may be used to image dynamic ADP-actin remodelling.

Accordingly, the present disclosure also provides a method for the detection and/or modulation of ADP-actin remodelling, or in drug screening of candidate modulators of ADP-actin remodelling, wherein the method may comprise (i) expressing a tagged recombinant polypeptide of the first aspect in a cell; (ii) detecting whether the tagged polypeptide associates with ADP-actin or F-actin using total internal reflection (TIRF) microscopy; and for drug screening, exposing the cell before step (ii) to candidate modulators of ADP-actin remodelling.

Other ADP-actin-associated cellular processes, such as but not limited to, cell movement/migration, cell shape, cell-matrix interaction, cell polarity, muscle development, cell division and/or differentiation may also be detected by means of the present invention. In some embodiments, the present invention may also be suitably adapted to detect glucose-starvation or other energy depletion conditions-related cellular processes, including aging and stress adaptation.

As described herein, the present invention may also be suitably applied for use in pharmaceutical research. Given that actin cytoskeleton plays an important role in practically all types of cellular morphogenesis, ADP-actin may be thus used as a marker or target in cell-based screening methods and/or therapeutic approaches. Accordingly, the present invention may be suitably applied for use in drug screening such as cell-based screens for drugs, or in the study of actin-associated diseases such as myopathies, familial thoracic aortic aneurysms, heart diseases and, more particularly, Baraitser-Winter syndrome. In some embodiments, the present invention may also be useful in the study and/or detection of cancer progression, aging progression, and microbial and/or viral infections in mammalian and/or plant hosts.

In some embodiments, the use is in-vitro. In some embodiments, the use is not in a human being.

In another aspect, there is provided a pharmaceutical composition comprising the recombinant polypeptide of the present invention, and a pharmaceutically acceptable carrier.

Suitable pharmaceutical carriers typically will contain inert ingredients that do not interact with the agent or active ingredient. Suitable pharmaceutical carriers for parenteral administration include, for example, sterile water, physiological saline, bacteriostatic saline (saline containing about 0.9% 10 mg/ml benzyl alcohol), phosphate-buffered saline, Hank's solution, Ringer's lactate and the like. Formulations can also include small amounts of substances that enhance the effectiveness of the active ingredient (e.g., emulsifying agents, solubilizing agents, pH buffering agents, wetting agents). Methods of encapsulating compositions (such as in a coating of hard gelatin or cyclodextran) are known in the art. For inhalation, the agent can be solubilized and loaded into a suitable dispenser for administration (e.g., anatomizer or nebulizer or pressurized aerosol dispenser).

In another aspect, there is provided a recombinant polypeptide, a polynucleic acid molecule, a recombinant cell or a pharmaceutical composition as disclosed in the present invention for use in medicine.

In another aspect, there is provided a use of a recombinant polypeptide, a polynucleic acid molecule or a recombinant cell as disclosed herein in cell-based screens for drugs or in the study of diseases associated with actin-remodelling, wherein the use is not in a human being.

In a further aspect of the disclosure, there is provided a method of treating a disease associated with actin-remodelling, comprising administering to a subject in need thereof, a therapeutically effective amount of a recombinant polypeptide or a pharmaceutical composition as disclosed herein, to a patient in need thereof.

In yet another aspect of the present disclosure, there is provided a use of a recombinant polypeptide or a pharmaceutical composition of the present invention in the manufacture of a medicament for the treatment of a disease associated with actin-remodelling.

In some embodiments, the disease associated with actin-remodelling may include but is not limited to the group comprising cancer, myopathies, familial thoracic aortic aneurysms, heart diseases such as hypertrophic cardiomyopathy (HCM), dilated cardiomyopathy (DCM), Baraitser-Winter syndrome and auto-inflammatory diseases. In some embodiments, the disease associated with actin-remodelling may be linked to energy depletion-related cellular processes, such as aging.

For in vivo delivery, the composition of the present disclosure can be delivered to a subject in need thereof by a variety of routes of administration including, for example, oral, nasal, dietary, topical, transdermal, or parenteral (e.g., intra-arterial, intravenous, intramuscular, subcutaneous injection, intradermal injection) routes of administration. Administration can be local or systemic. Preferably, the medicament is formulated for subcutaneous or intravenous administration. The actual dose of a therapeutic amount of the composition disclosed herein and treatment regimen can be determined by a skilled physician, taking into account the nature of the condition being treated, and patient characteristics.

Unless otherwise indicated or otherwise evident from the context and understanding of one of ordinary skill in the art, values that are expressed as ranges can assume any specific value or subrange within the stated ranges in various embodiments, to the tenth of the unit of the lower limit of the range, unless the context clearly dictates otherwise. “About” in reference to a numerical value generally refers to a range of values that fall within +10%, in some embodiments±5%, in some embodiments±1%, in some embodiments±0.5% of the value unless otherwise stated or otherwise evident from the context. In any embodiment in which a numerical value is prefaced by “about”, an embodiment in which the exact value is recited is provided. Where an embodiment in which a numerical value is not prefaced by “about” is provided, an embodiment in which the value is prefaced by “about” is also provided. Where a range is preceded by “about”, embodiments are provided in which “about” applies to the lower limit and to the upper limit of the range or to either the lower or the upper limit, unless the context clearly dictates otherwise. Where a phrase such as “at least”, “up to”, “no more than”, or similar phrases, precedes a series of numbers, it is to be understood that the phrase applies to each number in the list in various embodiments (it being understood that, depending on the context, 100% of a value, e.g., a value expressed as a percentage, may be an upper limit), unless the context clearly dictates otherwise. For example, “at least 1, 2, or 3” should be understood to mean “at least 1, at least 2, or at least 3” in various embodiments. It will also be understood that any and all reasonable lower limits and upper limits are expressly contemplated.

Having now generally described the invention, the same will be more readily understood through reference to the following examples which are provided by way of illustration, and are not intended to be limiting of the present invention.

### EXAMPLES

Standard molecular biology techniques known in the art and not specifically described were generally followed as described in Green and Sambrook, Molecular Cloning: A Laboratory Manual, Cold Springs Harbor Laboratory, New York (2012).

### Example 1: Materials and General Methods

### Yeast Culture and Plasmids

Yeast strains in S288C background, primers, and plasmids used in this work are listed in Table 2. Yeast strains were grown in standard-rich media or complete synthetic media as previously described (Miao et al., Nature comms 7, 11265 (2016); Xie, Y. et al., Nature comms 10, 1-18 (2019)). Genomic C-terminal fluorescent fusion tagging at the endogenous locus was performed using the lithium acetate-based method by transforming PCR-fragments derived from pFA6a-GFP (S65T)-kanMX or pFA6a-GFP (S65T)-His3MX6 (Longtine et al., Yeast 14, 953-961 (1998); Miao et al., Nature comms 7, 11265 (2016); Xie, Y. et al., Nature comms 10, 1-18 (2019)). The yeast strains expressing Spa2-truncating variants were generated from the integration vector pRS305 containing Spa2 genomic+500 bp and DNA fragment 741-1605 bp. Transformants were selected on the YPD agar plates containing 200 ug/ml G418 or synthetic complete media supplemented with appropriate amino acids. The information for yeast strains and primers used are indicated in Tables 2 and 3.

TABLE 2
Yeast strains used in this study

| | | |
|---|---|---|
| YMY2011 | MATa/MATα his3-D200/his3-D200ura3-52/ura3-52leu2-3, 112/leu2-3, 112 | Lab stock |
| | lys2- 801/LYS2 ade2-1/ADE2 | |
| YMY2012 | MATα his3-Δ200 leu2-3, 112 ura3-52 | Lab stock |
| YMY2130 | MATα his3-Δ200 leu2-3, 112, ura3-52, Abp140-3GFP::His3 | Lab stock |
| YMY2131 | MATa his3-Δ200 leu2-3, 112, ura3-52, spa2Δ::Leu2, Abp140-3GFP::His3 | Lab stock |
| YMY2132 | MATa his3-Δ200 leu2-3, 112, ura3-52, cbp2Δ::KanMX, Abp140-3GFP::His3 | This study |
| YMY2133 | MATa his3-Δ200 leu2-3, 112, ura3-52, hxk2Δ::KanMX, Abp140-3GFP::His3 | This study |
| YMY2134 | MATα his3-Δ200 leu2-3, 112, ura3-52, spa2Δ cbp2Δ::KanMX, Spa2-GFP::His3, | This study |
| | Abp140-Tomato::Ura3 | |
| YMY2135 | MATα his3-Δ200 leu2-3, 112, ura3-52, Spa2-GFP::His3, Abp140-Tomato::Ura3 | This study |
| YMY2136 | MATa his3-Δ200 leu2-3, 112, ura3-52, Queen::His3 | This study |
| YMY2137 | MATa his3-Δ200 leu2-3, 112, ura3-52, spa2Δ::Leu2, Queen::His3 | This study |
| YMY2138 | MATa his3-Δ200 leu2-3, 112, ura3-52, cbp2Δ::KanMX, Queen::His3 | This study |
| YMY2139 | MATa his3-Δ200 leu2-3, 112, ura3-52, cbp2Δ::KanMX, spa2Δ::Leu2, Queen::His3 | This study |
| YMY2140 | MATa his3-Δ200 leu2-3, 112, ura3-52, hxk2Δ::KanMX, Queen::His3 | This study |
| YMY2141 | MATα his3-Δ200 leu2-3, 112, ura3-52, bni1Δ::Leu2, Abp140-3GFP::His3 | This study |
| YMY2142 | MATα his3-Δ200 leu2-3, 112, ura3-52, bud6Δ::Ura3, Abp140-3GFP::His3 | This study |
| YMY2143 | MATα his3-Δ200 leu2-3, 112, ura3-52, bnr1Δ::KanMX, Abp140-3GFP::His3 | This study |
| YMY2144 | MATa his3-Δ200 leu2-3, 112, ura3-52, aip5Δ::Ura3, Abp140-3GFP::His3 | This study |
| YMY2145 | MATa his3-Δ200 leu2-3, 112, ura3-52, tpm1Δ::HIS3, Abp140-3GFP::His3 | This study |
| YMY2146 | MATα his3-Δ200 leu2-3, 112, ura3-52, myo2-66::KanMX, Abp140-3GFP::His3 | This study |
| YMY2147 | MATα his3-Δ200 leu2-3, 112, ura3-52, sac6Δ::Leu2, Abp140-3GFP::His3 | This study |
| YMY2148 | MATα his3-Δ200 leu2-3, 112, ura3-52, cap1Δ::Leu2, Abp140-3GFP::His3 | This study |
| YMY2149 | MATα his3-Δ200 leu2-3, 112, ura3-52, cap2Δ::Leu2, Abp140-3GFP::His3 | This study |
| YMY2150 | MATα his3-Δ200 leu2-3, 112, ura3-52, cof1-4::Leu2, Abp140-3GFP::His3 | This study |
| YMY2151 | MATa his3-Δ200 leu2-3, 112, ura3-52, gpr1Δ::KanMX, Abp140-3GFP::His3 | This study |
| YMY2152 | MATa his3-Δ200 leu2-3, 112, ura3-52, spa2Δ::KanMX, spa2-535::Leu2, | This study |
| | Abp140-3GFP::His3 | |
| YMY2153 | MATa his3-Δ200 leu2-3, 112, ura3-52, spa2Δ::KanMX, spa2-281-535::Leu2, | This study |
| | Abp140-3GFP::His3 | |
| YMY2154 | MATa his3-Δ200 leu2-3, 112, ura3-52, spa2Δ::KanMX, spa2-281::Leu2, | This study |
| | Abp140-3GFP::His3 | |
| YMY2155 | MATa his3Δ1, leu2Δ0, met15Δ0, ura3Δ0, act1-101::KanM | Lab stock |
| YMY2156 | MATa his3Δ1 leu2Δ0 met15Δ0 ura3Δ0, act1-159::KanMX | Lab stock |
| YMY2157 | MATa his3Δ1 leu2Δ0 met15Δ0 ura3Δ0, act1-125::KanMX | Lab stock |
| YMY2018 | MATa his3-Δ200 leu2-3, 112, ura3-52, Spa2-GFP::His3 | Lab stock |
| YMY2158 | MATa his3-Δ200 leu2-3, 112, ura3-52, cbp2Δ::KanMX, Spa2-GFP::His3 | This study |
| YMY2159 | MATa his3-Δ200 leu2-3, 112, ura3-52, hxk2Δ::KanMX, Spa2-GFP::His3 | This study |
| YMY2160 | MATa his3-Δ200 leu2-3, 112, ura3-52, gpr1Δ::KanMX, Spa2-GFP::His3 | This study |
| YMY2161 | MATa his3-Δ200 leu2-3, 112, ura3-52, snf3Δ::KanMX, rgt2Δ::KanMX, | This study |
| | Spa2-GFP::His3 | |
| YMY2162 | MATα his3-Δ200 leu2-3, 112, ura3-52, bni1Δ::Leu2, Spa2-GFP::His3 | This study |
| YMY2163 | MATα his3-Δ200 leu2-3, 112, ura3-52, bud6Δ::Ura3, Spa2-GFP::His3 | This study |
| YMY2164 | MATα his3-Δ200 leu2-3, 112, ura3-52, bnr1Δ::KanMX, Spa2-GFP::His3 | This study |
| YMY2165 | MATa his3-Δ200 leu2-3, 112, ura3-52, aip5Δ::Ura3, Spa2-GFP::His3 | This study |
| YMY2166 | MATa his3-Δ200 leu2-3, 112, ura3-52, tpm1Δ::HIS3, Spa2-GFP::His3 | This study |
| YMY2167 | MATα his3-Δ200 leu2-3, 112, ura3-52, myo2-66::KanMX, Spa2-GFP::His3 | This study |
| YMY2168 | MATα his3-Δ200 leu2-3, 112, ura3-52, sac6Δ::Leu2, Spa2-GFP::His3 | This study |
| YMY2169 | MATα his3-Δ200 leu2-3, 112, ura3-52, cap1Δ::Leu2, Spa2-GFP::His3 | This study |
| YMY2170 | MATα his3-Δ200 leu2-3, 112, ura3-52, cap2Δ::Leu2, Spa2-GFP::His3 | This study |
| YMY2171 | MATα his3-Δ200 leu2-3, 112, ura3-52, cof1-4::Leu2, Spa2-GFP::His3 | This study |
| YMY2172 | MATa his3-Δ200 leu2-3, 112, ura3-52, Spa2-535-GFP::His3 | This study |
| YMY2173 | MATa his3-Δ200 leu2-3, 112, ura3-52, Spa2-281-GFP::His3 | This study |
| YMY2174 | MATa his3-Δ200 leu2-3, 112, ura3-52, spa2Δ::Leu2, Spa2-281-535-GFP::Ura3 | This study |
| YMY2175 | MATa his3-Δ200 leu2-3, 112, ura3-52, Spa2-1306-GFP::His3 | This study |
| YMY2176 | MATa his3-Δ200 leu2-3, 112, ura3-52, Spa2-1087-GFP::His3 | This study |
| YMY2177 | MATa his3-Δ200 leu2-3, 112, ura3-52, Spa2-816-GFP::His3 | This study |
| YMY2178 | MATa his3-Δ200 leu2-3, 112, ura3-52, cbp2Δ::KanMX, Spa2-535-GFP::His3 | This study |
| YMY2179 | MATa his3-Δ200 leu2-3, 112, ura3-52, hxk2Δ::KanMX, Spa2-535-GFP::His3 | This study |

TABLE 3
Primers used in this study

| SEQ ID NO: | Primer | DNA Sequence |
|---|---|---|
| 16 | Spa2-535-GFP-F | CGAAAATAAAATCCGATTCAAATGGTGAGAG |
| | | CACCACCTCGGATCCCCGGGTTAATTAA |
| 17 | Spa2-535-GFP-R | CTTTGTCTTCCTTTTCTTTCTCCTCTAGATAC |
| | | TACTAACTGAATTCGAGCTCGTTTAAAC |
| 18 | Spa2-281-GFP-F | TCCCGAACAGTTGAAGAGCCCTGAAGTACA |
| | | ACGGGCTGAGCGGATCCCCGGGTTAATTAA |
| 19 | Spa2-816-GFP-F | AACTACCTGCAAATATAGTGGAACTTGATTT |
| | | ACATGAGCGGATCCCCGGGTTAATTAA |
| 20 | Spa2-1087-GFP-F | CCATCTTCAGCTACACTGAAAAAGAGCGGG |
| | | CTCCCACGGATCCCCGGGTTAATTAA |
| 21 | PRS306-Spa2- | GCGGTGGCGGCCGCTCTAGAGTATACATAT |
| | pro-F | AATCACGTAA |
| 22 | PRS306-Spa2- | GTTTACTCTTTCTTTTCTTTTGTTTATTCTGTT |
| | pro-R | TCGGTGG |
| 23 | PRS306 | AAAGAAAAGAAAGAGTAAACGAGAACAATA |
| | Spa2-281-535-F | ACCCTAACTC |
| 24 | PRS306-Spa2-281- | GGATCCGTCGACCTGCAGCGGAGGTGGTG |
| | 535-R | CTCTCACCATT |
| 25 | PRS306-GFP-F | ATTCAAATGGTGAGAGCACCACCTCCGCTG |
| | | CAGGTCGAC |
| 26 | PRS306-GFP-R | GAGTTAGGGTTATTGTTCTCGTTTACTCTT |
| | | TCTTTTCTTT |

### Live-Cell Fluorescence Imaging

Yeast strains were cultured overnight at 30° C. or 25° C. in the synthetic complete media without tryptophan and re-inoculated into fresh medium to a final OD600=0.15 next morning for an additional 3 h of culture before imaging. Cells were immobilized onto Concanavalin A (ConA, 1 mg/ml)-coated coverslips and imaged by Spinning disc confocal (SDC) microscopy and super resolution SDC-structured illumination microscopy (SDC-SIM) were performed on a setup built around a Nikon Ti2 inverted microscope equipped with a Yokogawa CSU-W1 confocal spinning head, a Plan-Apo objective (100×1.45-NA), a back-illuminated sCMOS camera (Prime95B; Teledyne Photometrics), and a super resolution module (Live-SR; GATACA Systems) based on structured illumination with optical reassignment and image processing improvement (Roth and Heintzmann, Methods Appl Fluoresc 4, 045005 (2016)). The method, known as multifocal structured illumination microscopy (York et al., Nat Methods 9, 749-754 (2012)), allows combining the doubling of the resolution with the optical sectioning capability of confocal microscopy. The maximum resolution is 128 nm with a pixel size in super resolution mode of 64 nm. Excitation light was provided by 488-nm/150 mW (Vortran) (for GFP), and all image acquisition and processing was controlled by MetaMorph (Molecular Device) software. The images were acquired continuously at a 0.25-μm interval for a total range of 7.5 μm in the z-direction, using an exposure time of 200 ms for actin cable, 100 ms for Spa2 localization, and 2x binning.

### Energy Starvation

To examine the pattern Spa2 localization in vivo and actin cables in response to ES, early log-phase cells were treated by adding SM without glucose or adding 20 mM 2-DG (inhibition of glycolysis) and 10 UM antimycin A (inhibits mitochondrial ATP production).

### Queen-Expressing Strains, Imaging, and Analysis

Queen plasmid used was purchased from NBRP Yeast Genetic Resource Center (NBRP/YGRC) (Tanaka et al., Nat Commun 9, 1860 (2018)). Queen-expressing budding yeast strains were generated by linearizing the constructs by Pstl and then inserting them into the his3Δ1 locus of the YMY2012 and glucose relevant mutant strains hxk2Δ, gpr1Δ, snf3Δrgt2Δ. Budding yeast cells were immobilized on the glass cover slip-coated with concanavalin A at 1 mg/ml before imaging, the immobilized cells were imaged using a Spinning disc confocal (SDC) microscopy on a setup built around a Nikon Ti2 inverted microscope equipped with a Yokogawa CSU-W1 confocal spinning head, a Plan-Apo objective (100×1.45-NA), a back-illuminated sCMOS camera (Prime95B; Teledyne Photometrics), Imaging lasers were provided by 405 nm/100 mW (Vortran), 488 nm/150 mW (Vortran), combined in a laser launch (iLaunch, GATACA Systems). Images of cells were acquired from several fields of view for providing enough sample for quantitative analysis. The QUEEN ratio was calculated using Fiji software (Schindelin et al., Nat Methods 9, 676-682 (2012)) and was calculated as follows. First, both the QUEEN images were converted to signed 16-bit floating-pointed grayscale and the QUEEN signals in cells were corrected for background by subtracting the mean pixel values from the signal outside the cells, next The pixel values of the ex405 image were divided by those of the ex480 image to calculate the QUEEN ratio at each pixel. The mean ratio in pixels was used to represent the ATP level in cells.

### Total Internal Reflection Fluorescence Microscopy

For TIRFM experiments, 25×50-mm coverslips (Marienfeld Superior) were cleaned with 20% sulfuric acid overnight and rinsed thoroughly with sterile water. The coverslips were then coated with 2 μg/ml biotin-PEG-silane (Laysan Bio Inc.) in 80% ethanol (pH 2.0, adjusted by HCl) and 2 mg/ml methoxy-PEG-silane at 70° C. for overnight. The next day, coverslips were rinsed thoroughly with sterile water and dried in nitrogen stream and kept at −80° C. before use. To prepare TIRF imaging chambers, the functionalized coverslip was attached to a plastic flow cell chamber (Ibidi, sticky-Slide VI 0.4), followed by a 30 s incubation with HBSA buffer (20 mM Hepes, pH 7.5, 1 mM EDTA, 50 mM KCl, and 1% bovine serum albumin) and then 60 s incubation with 0.1 mg/ml streptavidin in HEKG10 (20 mM Hepes, pH 7.5, 1 mM EDTA, 50 mM KCl, 10% [vol/vol] glycerol). Then, the flow cell chamber was washed by TIRF buffer (10 mM imidazole, 50 mM DTT, 15 mM glucose, 50 mM KCl, 1 mM MgCl₂, 1 mM EGTA, 100 μg/ml glucose oxidase, and 0.5% methylcellulose [4000 cP], 0.3 mM ADP or ATP, pH 7.4). Recombinant protein prepared in TIRF buffer were mixed with 3 μM G-ADP-actin or 0.5 UM G-ATP-actin (10% Oregon Green 488 labeled, 0.5% biotin labeled) before flowing into the chamber. Time-lapse images were acquired at room temperature at 5-s intervals for 10 min or 60 min with the above-mentioned spinning-disc confocal system with TIRF module (iLasV2 Ring TIRF, GATACA Systems). For actin elongation rate quantification, the fast elongation end of individual filament was traced by hand for a time period of 2 min each. We used the conversion factor of 370 subunits per micrometre of F-actin to calculate the elongation rate.

### Actin Anisotropy Assay

For measurements of Spa2 with actin monomer, fluorescent proteins diluted to 60 nM (fluorescein Alexa488-labeled Spa2-281, 281-535, 535) in 20 mM Hepes 500 mM Nacl buffer. Stock of non-polymerizable monomeric actin (Hypermol, Germany) was titrated down starting from 50 μM, from which dilutions were further made. Equal volumes of Aleax-488-Spa2 and G-actin were incubated at least 2 hr in dark at 25° C. Alexa 488 fluorescence (485/510 nm) was measured by the plate reader Synergy™ H4 (BioTek, USA). Total volume was 25 μl containing 30 nM Spa2 protein.

### In Vivo Actin Cable Imaging and Image Analysis

For comparing actin cable intensity, the immobilized cells were imaged by SDC-SIM with a Plan-Apo objective (100×1.45-NA), a back-illuminated sCMOS camera (Prime95B; Teledyne Photometrics), and a super resolution module (Live-SR; GATACA Systems) based on structured illumination with optical reassignment and image processing improvement (Roth and Heintzmann, Methods Appl Fluoresc 4, 045005 (2016)). The method, known as multifocal structured illumination microscopy (York et al., Nat Methods 9, 749-754 (2012)), allows combining the doubling of the resolution with the optical sectioning capability of confocal microscopy. The maximum resolution is 128 nm with a pixel size in super resolution mode of 64 nm. Excitation light was provided by 488-nm/150 mW (Vortran) (for GFP), 561-nm/100 mW (Coherent) (for mCherry/mRFP/tagRFP) and all image acquisition and processing was controlled by MetaMorph (Molecular Device) software. Each of condition cells were cropped, background was subtracted from average projections in Fiji. Individual cables in average projections of mother cells were traced in Fiji using a line that encompassed the entire width of the cable. The mean fluorescence signal intensity per filament and total fluorescence actin cable intensity per cell was measured as previously described (Garabedian M. V. et al., J cell Biol. 217, (2018)). The mean intensity per filament and total intensity per cell after ES were normalized with glucose control.

### (i) Surface Plasmon Resonance (SPR)

SPR experiment was performed at room temperature in buffer containing 20 mM HEPES and 50 mM NaCl pH 7.4 using Biacore T200 instrument (GE Healthcare). Recombinant Spa2 variants were immobilized on the CM5 chip (GE Healthcare) by amine coupling. The carboxyl group on the dextran surface of the chip was converted to amine-reactive ester by reacting with 0.2 M 1-ethyl-3-(3-dimethylpropyl)-carbodiimide and 0.1 M N-hydroxysuccinimide. The molecule flowing over the sensing surface were performed by injection at a flow rate of 10 ml/min at pH 4.5, while the reference cell was left blank without the injected protein. To test the binding of analyte Spa2 with the immobilized ligand Spa2 protein variants, serially diluted (1:1) Spa2 variant was flown in over the surface of the control and ligand for 60 s and dissociated with buffer (20 mM HEPES, 50 mM NaCl, pH 7.4) for 150 s at a rate of 30 μl/min. Spa2 was injected at gradient concentrations as 20, 10, 5, 2.5, 1.25, 0.625, 0.3125, and 0.15625 μM. The chip surface with left-over protein captured was regenerated by treating with 50 mM NaOH for 3 s at 100 μl/min after each cycle. The kinetics of binding was analyzed by the Biacore T200 Evaluation software (GE Healthcare). The sensorgram for the binding experiment was normalized with the reference cell and fitted to the bivalent analyte model using Biacore T200 Evaluation software (GE Healthcare).

### ii) Recombinant Protein Expression and Purification

Spa2-281, Spa2-281-535, Spa2-535 and Spa2-281-535-trimer proteins were expressed and purified from Escherichia coli (BL21 (DE3) Rosetta T1R). Cells were cultured 2 L TB medium (24 g yeast extract per liter, 20 g tryptone per liter, 4 ml glycerol per liter, phosphate buffer pH 7.4) containing 50 μg/ml of Kanamycin at 37° C. to an OD600 of 0.6 before induction by 0.5 mM IPTG at 16° C. overnight. The cells were harvested by centrifugation at 4° C., 5000×g (rotor JA10) for 15 min. The pellet was resuspended in 20 mM Hepes PH 7.4 and 500 mM NaCl, 20 mM Imidazole, 1 mM PMSF, protease inhibitor Cocktail Set III, EDTA free from Thermo Fisher and lysed by LM20 microfluidizer (20000 psi). The lysate was clarified by centrifugation at 25,000×g for 1 h using rotor JA25.5 (Beckman Coulter). The supernatant was purified by FPLC AKTAxpress system (GE Healthcare) using Ni-NTA affinity chromatography. The elution fractions containing targeted proteins from gradient elution with increasing imidazole concentration were pooled and further purified by size-exclusion chromatography using a HiLoad 16/600 Superdex75 column (GE Healthcare) in 20 mM Hepes and 500 mM NaCl. Proteins were flash-frozen in liquid N2 prior to storage at −80° C. in small aliquots.

To obtain monomeric Ca²⁺-ATP-actin with or without labelling with Oregon Green™ 488 Iodoacetamide (ThermoFisher) or NHS-dPEG®4-biotin (Sigma), 5 g of rabbit muscle acetone powder (Pel-Freez Biologicals) was dissolved in 60 ml of G-buffer (5 mM Tris pH 8.0, 0.2 mM ATP, 0.1 mM CaCl₂), 0.5 mM dithiothreitol (DTT)) for 30 min at 4° C. The mixture was filtered through cheese cloth three times to collect the actin-rich extracts in supernant. The filtration was repeated for three times and the actin-rich extracts were combined and subjected to centrifugation at 18,000×g by rotor JA-25.5 (Beckman). Afterwards, the clear actin-rich supernant added 50 mM KCl and 2 mM MgCl₂ to allow actin polymerization with slow stirring at 4° C. for 1 h. To remove F-actin binding proteins, KCl powder was used to add slowly until a final concentration of 0.8 M that was subsequently stirred slowly for an additional 30 min. The solution was subjected for 95,800×g centrifugation for 3 h with rotor Ti45 (Beckman) at 4° C. to collect the polymerized F-actin. F-actin pellet was rinsed with 1 ml G-buffer and all the F-actin pellets were transferred to the 10 ml homogenizer with 5 ml G-buffer and the pellet was homogenized by moving up and down. To further depolymerize the F-actin, 4 cycles of sonication with 3 s on and 10 s off were applied. F-actin were then dialyzed against 1 liter of G-buffer without DTT overnight. The next day, we changed to 1 liter of new G-buffer without DTT and kept on dialysis. In the meanwhile, we freshly prepared the Oregon Green™ 488 lodoacetamide or NHS-dPEG® 4-biotin with high-quality dimethylformamide into a final concentration of 10 mM. The dialyzed actin was determined by OD290 using nanodrop. Before proceeding to actin labeling, we diluted the actin with cold 2x labeling buffer (50 mM Imidazole pH 7.5, 200 mM KCl, 0.3 mM ATP, 4 mM MgCl₂) until a actin concentration at 23 μM. 12-15-fold molar excess of Oregon Green™ 488 lodoacetamide/NHS-dPEG®4-biotin stock were added dropwise with very gentle vortex. To allow sufficient labeling, we kept the solution in the dark with aluminium foil and rotated at 4° C. overnight. The next day morning, we pelleted the labeled actin with Type 50.2 rotor (Beckman) at 111,000×g for 3 h. We then collected all the pellets and transferred to the homogenizer with 5 ml G-buffer. Afterwards, the actin was homogenized for 20 times and sonicated with 4 cycles of 3 s on and 10 s off. To further depolymerize the actin, we dialyzed against 1 liter of G-buffer for at least 2 times (24-36 h). We then applied the dialyzed actin to centrifugation at 167,000×g for 2.5 h with rotor SW55 Ti (Beckman) at 4° C. To further purify the actin, we collected the ⅔ of the supernatant on top and injected to fast protein liquid chromatography system, separated through column HiPrep™ 16/60 Sephacryl™ S-300 HR. The collected labeled actin was dialyzed overnight against G-buffer with 50% glycerol to reduce the total volume. Small aliquots of actin were prepared and freshly frozen by liquid N2 for long-term storage.

### In Vitro Protein Condensation Assay and Imaging

Recombinant Spa2 variants (10% Alexa488-labeled) were incubated for 5 min at room temperature before applying 5 μl on the coverslip and being imaged by SDC-SIM. A serial dilution of protein concentration and ionic strength were performed by starting from the high concentration proteins examined at 500 mM NaCl after protein purification.

### Analytical Ultracentrifugation (AUC)

AUC Sedimentation Velocity (AUC-SV) experiments were performed on Beckman Proteome Lab XL-I Analytical Ultracentrifuge using an 8-hole An-50 Ti analytical rotor. Samples were dialyzed overnight in buffer (20 mM Hepes, pH 7.4, 50 mM NaCl) and loaded into 2-sector cells fitted with 1.2 cm epon centerpiece and quartz windows. The samples were centrifuged at 30,000 rpm or 45,000 rpm at 20° C. and absorbance at 280 nm was recorded every 5-10 minutes during 15 hours centrifugation. The data were analyzed with SEDFIT software using c(s) and c (s,ff0) size distribution models, and plotted with GUSSI software. Sedimentation coefficients were standardized to s20,w using the partial specific volume of the proteins (calculated using SEDFIT software), solvent density, and viscosity (calculated using SEDNTERP software).

### ADP-G-Actin Preparation

A 100 ml ATP-G-actin adding 1 ml 100×ME (50 UM MgCl₂ and 250 μM EGTA) and incubate for 30 min on ice. Add 1 μl glucose (1 mM) and 5.4 μl Hexokinase (0.02 units/μl), mix for 3 h at cold room. Centrifuge 80K 55 min at 4 degrees (TLA, Ultra Centrifuge Rotor from Beckman), got top 80% supernatant and measure the concentration (A290=0.0266/μM/cm).

### In Vitro Actin Polymerization and Imaging

A 10 μM G-actin prepared in G-buffer was converted to Mg2+-ATP-actin or Mg2+-ADP-actin on ice for 5 min before being mixed with the examined recombinant proteins in the G buffer.

The actin polymerization was initiated by adding 10×KME (500 mM KCl, 10 mM MgCl₂, and 10 mM EGTA) buffer mix in a total reaction volume of 50 μl for 30 min at room temperature. Five 10 μl polymerized actin sample was incubated with acti-stain 488- or acti-stain 565-phalloidin (Cytoskeleton, Inc.) at a final concentration of 0.5 μM ATP-actin or 3 μM ADP-actin for 5 min before being diluted by F-Buffer (G-buffer plus 1×KME) and applied on the poly-lysine (0.01%)-coated coverglass for microscopic imaging using a ×100 oil objective lens.

### Protein Sequence Prediction and Conservative Analysis

To identify the Spa2-535 homologs and perform conservative analysis, the query Spa2-535 sequence was submitted in FungiDB (https://fungidb.org/fungidb/) with default parameters. The top 100 hits of Aip5 homologs were chosen from the species. Their correspondent sequence alignment was performed in the online server Clustal Omega (https://www.ebi.ac.uk/Tools/msa/clustalo/) and the phylogenetic tree was generated by the interactive tree of life (http://itol.embl.de/). ANCHOR (http://anchor.enzim.hu/) and PHYRE2 protein fold recognition server (http://www.sbg.bio.ic.ac.uk/~phyre/) were used, respectively, in identifying the unstructured and structured domains. Structures of the ScSpa2-535 homologs in other fungi species were performed by Alphafolder2 software (Jumper et al., Nature 596, 583-589 (2021)).

### U2OS Cell Growth and Energy Starvation

U2OS cell were used for Lipofectamine transfection. Cells were grown in DMEM (high glucose, GlutaMAX™, Gibco) supplemented with 10% fetal bovine serum (GE HyClone), 100 units/ml penicillin and 100 μg/ml streptomycin at 37° C. in a CO₂ incubator. To generate plasmids for overexpressing C-terminal GFP-tagged Spa2 variant truncations (pEGFP-N1-Spa2) in U2OS, sequences encoding yeast Spa2 were amplified by PCR using cDNA clones from yeast Spa2 as templates and inserted into the pEGF-N1 vector. U2OS were transiently transfected with the plasmids described above using Lipofectamine 3000 (Invitrogen, USA) following the manufacturer's protocol and grown overnight on around glass-bottom dish (Thermo Fisher) at 37° C. in a CO₂ incubator for protein expression. Transfected cells were treated with DMEM including 0.45% 2-DG but no glucose for 10 min, then fix cells and stained with phalloidin 565.

### Statistical Analysis

All statistical analyses were performed using GraphPad Prism 6 software. p Values were determined by two-tailed Student's t test assuming equal variances and the one-way analysis of variance (*p<0.05, ** p<0.01, *** p<0.001, **** p<0.0001, and ns=no significant). Error bars indicate the standard deviation (S.D.). See Table 2 for an overview of the used sources.

### Example 2: Spa2 is Responsible for Actin Cable Stabilization Upon Energy Starvation Via the N-Terminus

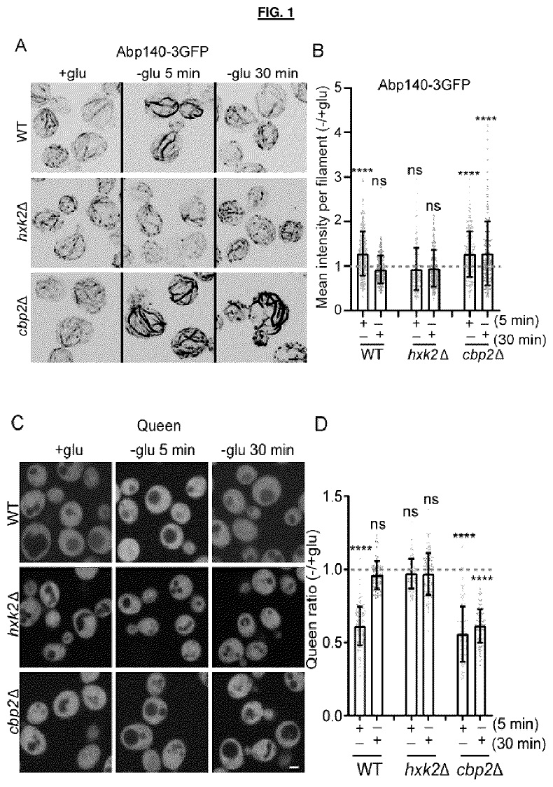

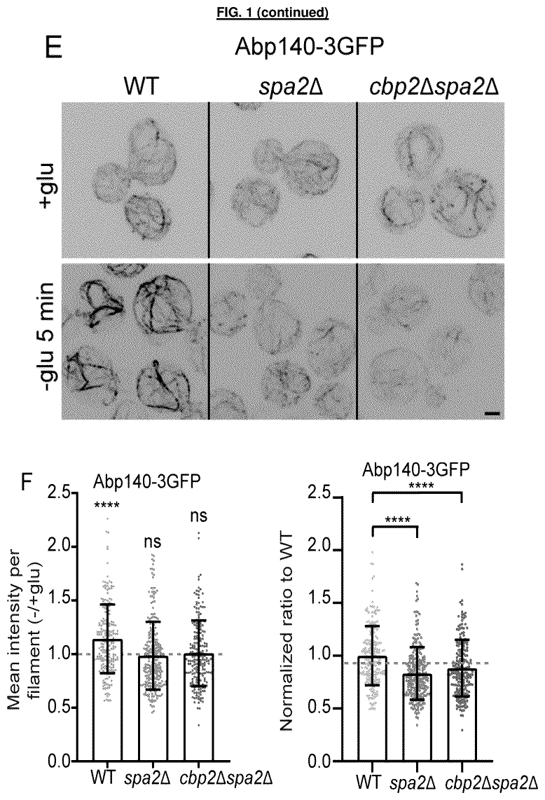

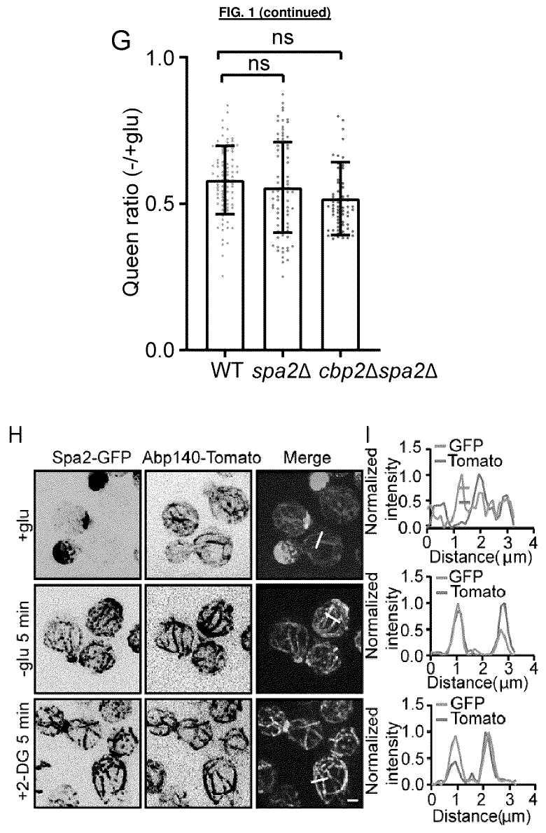

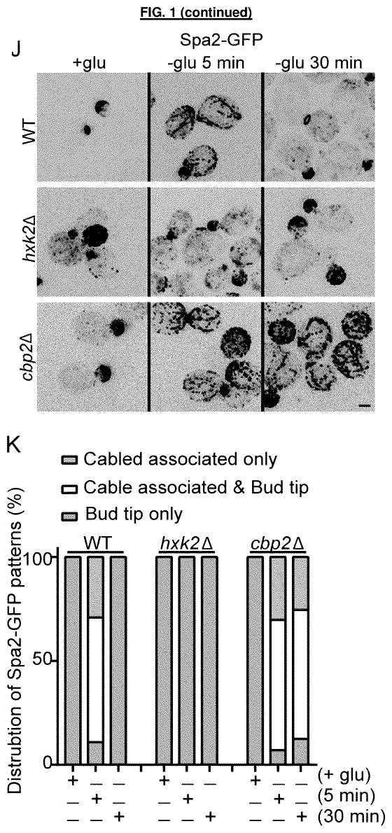

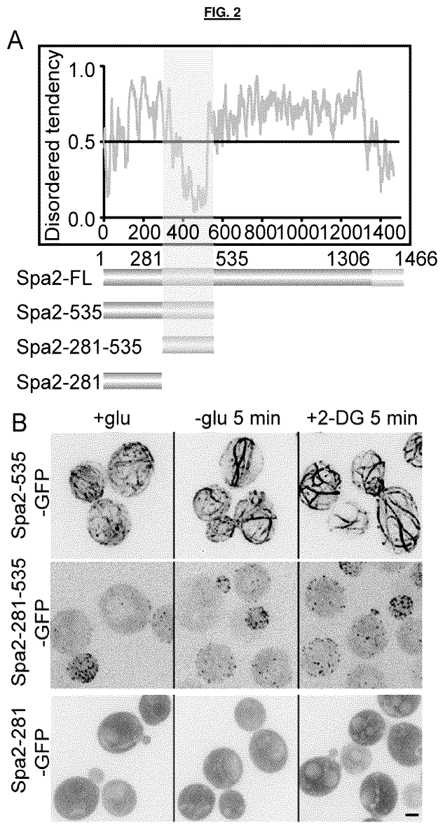

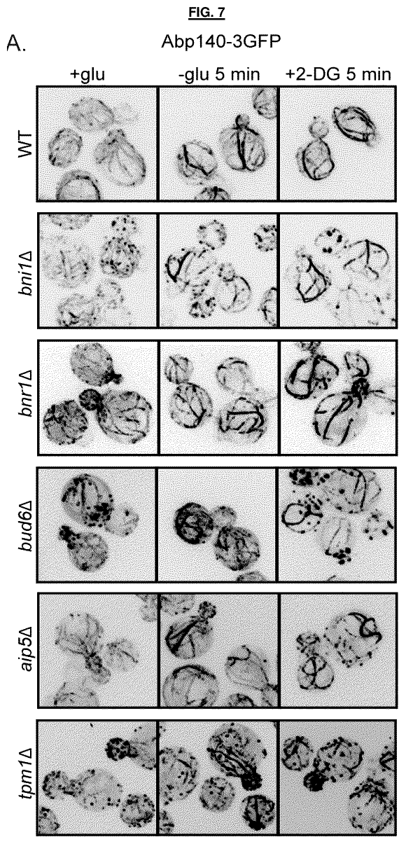

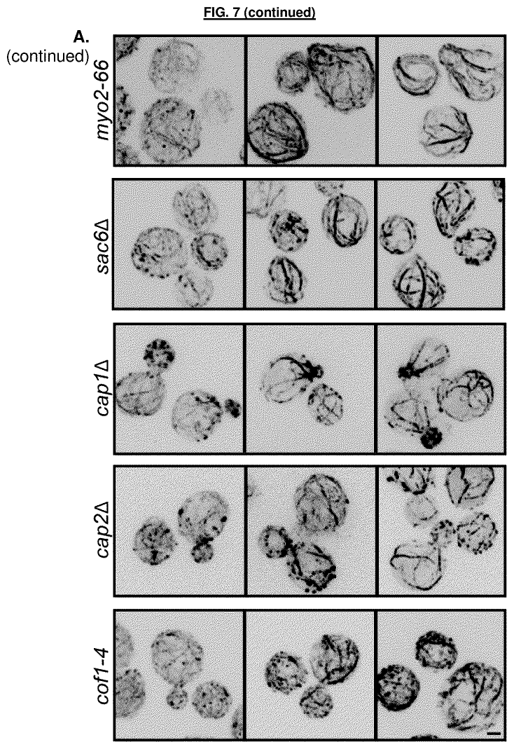

Upon glucose starvation (GS), Abp140-3GFP-marked actin cables in budding yeast showed time-dependent oscillation with rapid bundling at 5 min and recovery at 30 min (FIGS. 1A and 1B). The spatially regulated actin cable remodelling was consistent with the initial drop in the ATP: ADP ratio by ~38% at 5 min and the restoration at 30 min (FIGS. 1C and 1D). The GS-triggered rapid actin remodelling at 5 min was abolished in the major glucose kinase mutant hxk2Δ, in which the change in the ATP: ADP ratio was no longer sensitive to rapid GS (FIGS. 1A to 1D). Similarly, the anaerobic respiration mutant cbp2Δ was also unable to reverse GS-induced actin bundling at 30 min, when ATP could not be replenished (Xu, L., and Bretscher, A., Curr Biol 24, 2471-2479 (2014); Weber, C. A., et al., Proc Natl Acad Sci USA 117, 12239-12248 (2020)) (FIGS. 1A to 1D). In addition, it was found that overall actin cable production was also enhanced by GS (~2-fold) (FIG. 6A), which was greater than the bundling increase per cable (FIG. 1B), suggesting enhanced actin cable polymerization in addition to bundling. Additional ATP: ADP decrease conditions were also examined by using the glucose analog 2-deoxyglucose (2-DG). 2-DG diminishes the glycolytic supply of ATP and aerobic respiration-regenerated ATP in all WT, hxk2Δ, and cbp2Δ strains, in which actin cables were all stabilized without recovery over time (FIGS. 6B to 6D). In addition, in the ATP-production-uncoupled mutants of the G-protein-coupled glucose receptor gpr1Δ and glucose sensors snf3Δrgt2Δ, the changes in the ATP: ADP ratio and actin cable bundling were similar to those in the WT (FIGS. 6E to 6H).

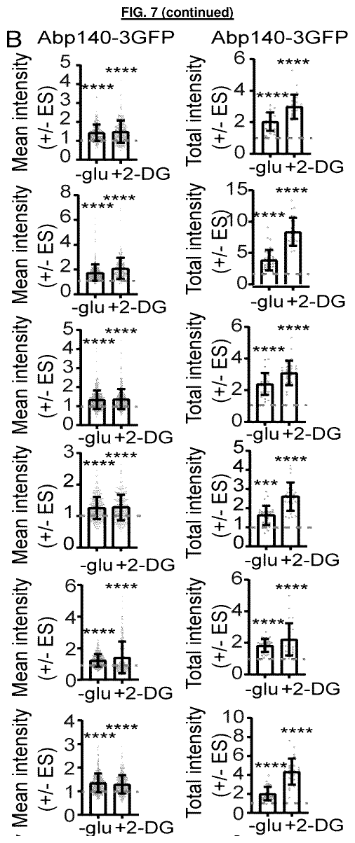

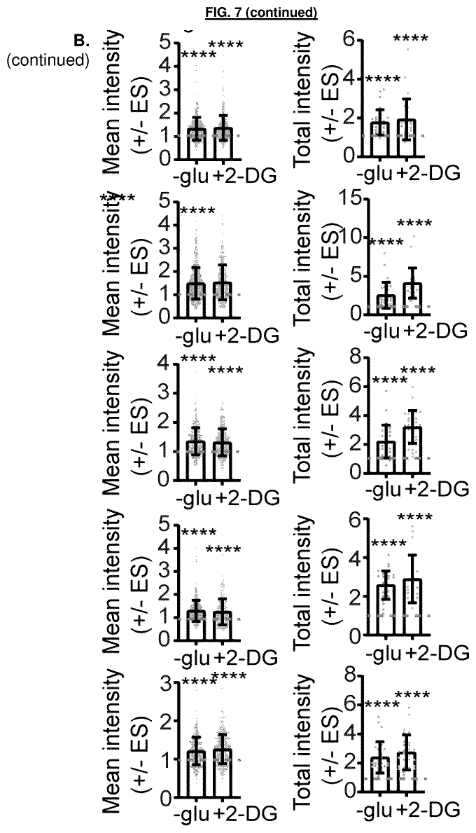

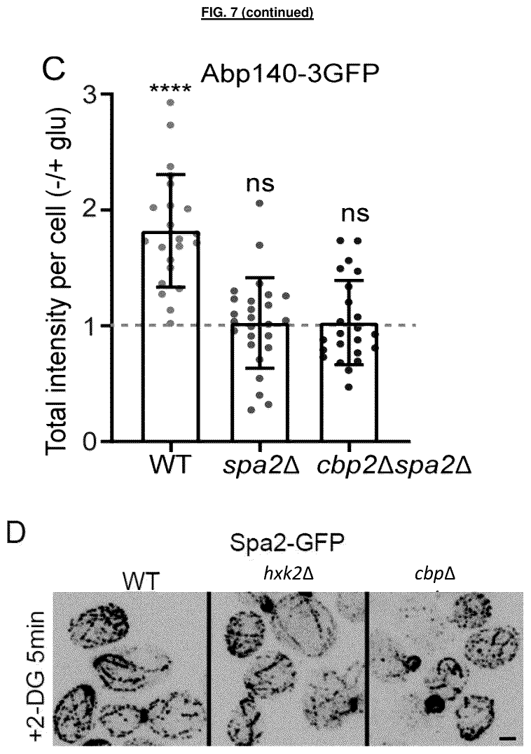

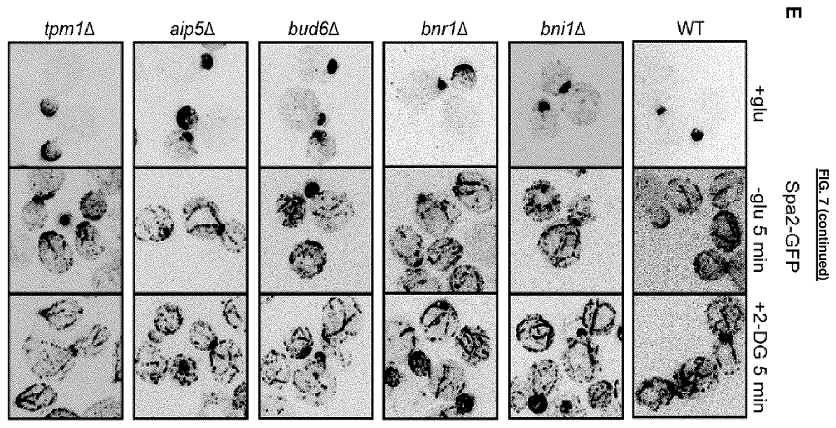

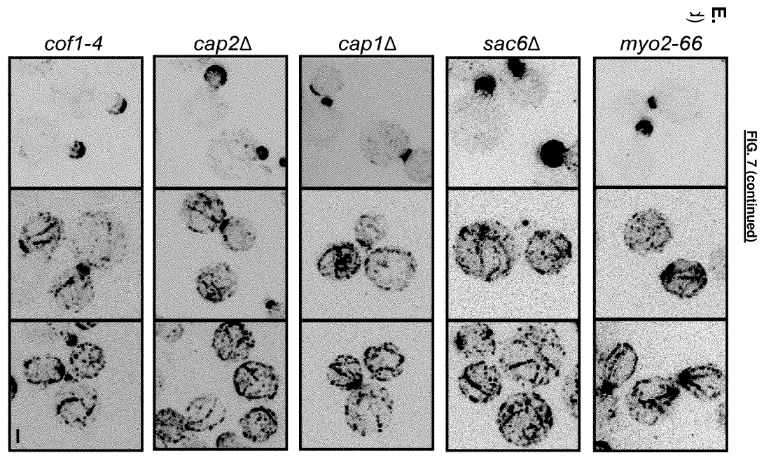

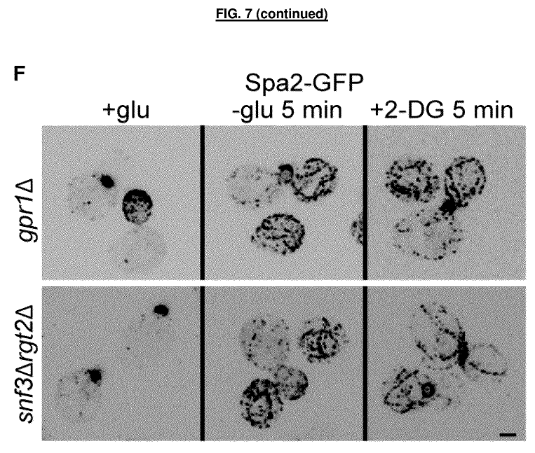

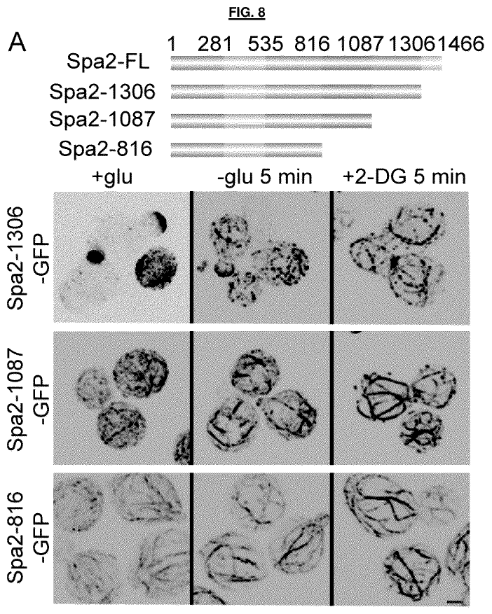

Next, the inventors sought to identify the potential regulatory actin-binding protein (ABP) by screening ABP mutants under energy starvation(ES), both GS and 2-DG. The inventors characterized Abp140-3GFP in mutants that were defective in actin cable nucleation (bni1Δ, bnr1Δ, bud6Δ, and aip5Δ), capping (cap1Δ and cap2Δ), actin cable crosslinking (tpm1Δ and myo2-66) and depolymerization (cof1-4). However, none of the above-listed mutants abolished ES-enhanced actin cable production and bundling (FIGS. 7A and 7B), except for a mutant knocking out SPA2, which encodes a polarisome complex scaffolding protein (Xie, Y. et al., Nature comms 10, 1-18 (2019); Xie, Y., and Miao, Y., J Cell Sci 134 (2021)). The actin cables in spa2Δ or spa2Δcbp2Δ were insensitive to GS (FIGS. 1E and 1F; FIG. 7C), although a similar decrease in the ATP: ADP ratio remained as in the WT (FIG. 1G).

Next, the inventors investigated whether Spa2 localizes on the actin cable during ES by imaging Spa2-GFP localization. After 5 min of ES, bud tip-localized Spa2-GFPs are depolarized and form filamentous structures or puncta along the filaments, which largely overlap with Abp140-Tomato-labeled actin cable bundles (FIGS. 1H and I). While polarized Spa2-GFP relocalized to actin cables from the cortex after 5 min of GS in WT, filamentous Spa2-GFP could not form in hxk2Δ or was restored to the bud tip in cbp2Δ after 30 min (FIGS. 1J and 1K; FIG. 7D). Except for spa2Δ, all the examined ABP mutants and glucose-related mutants (cbp2Δ, gpr1Δ, and snf3Δrgt2Δ) exhibited a similar response to the WT, forming Spa2-GFP filaments upon ES, similar to actin cable bundling (FIGS. 7E and 7F).

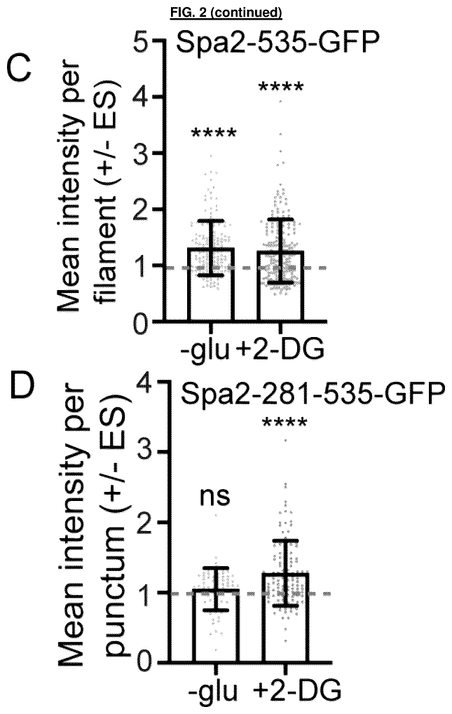

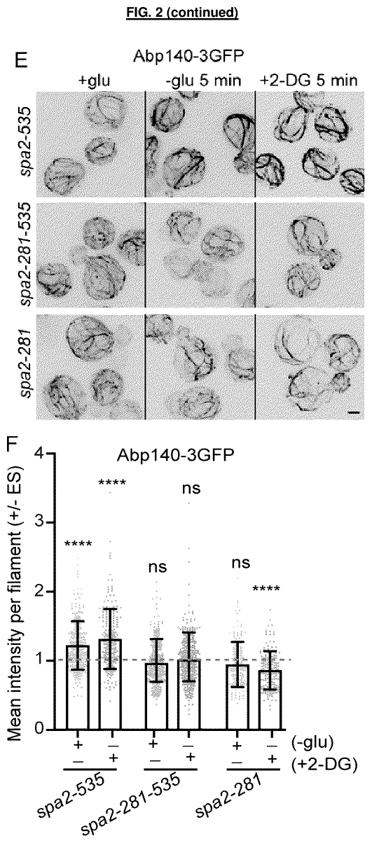

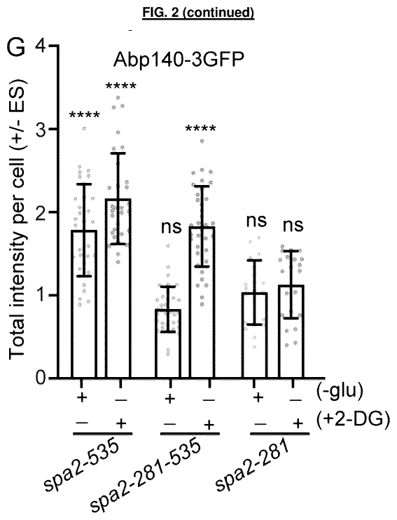

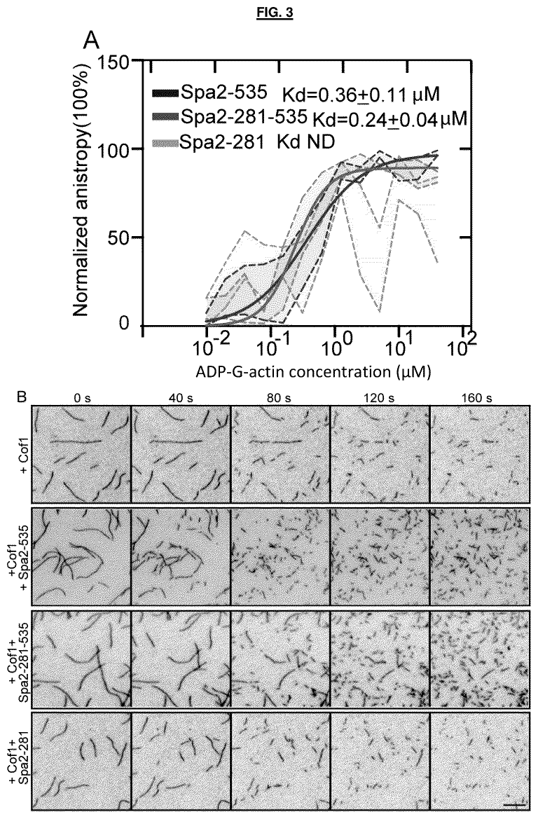

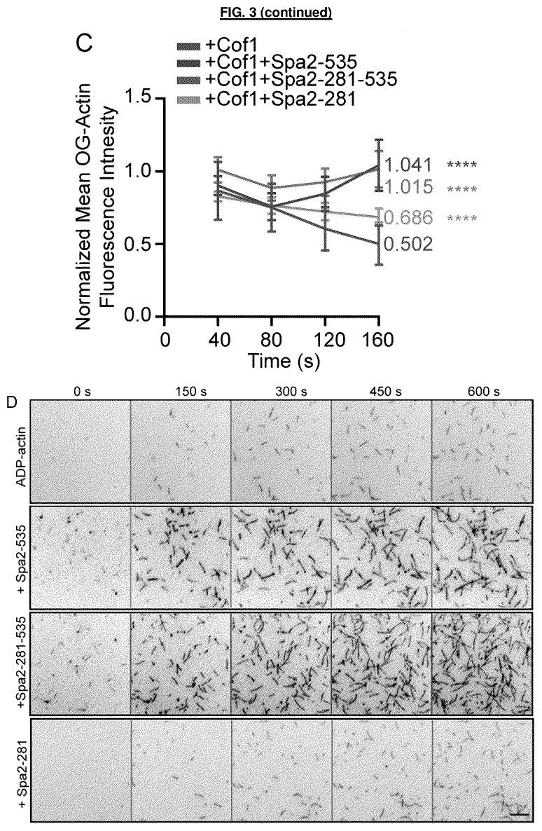

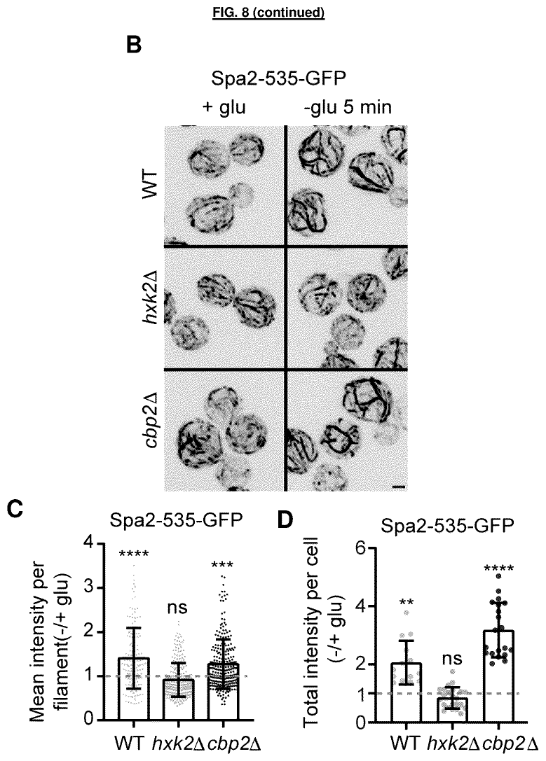

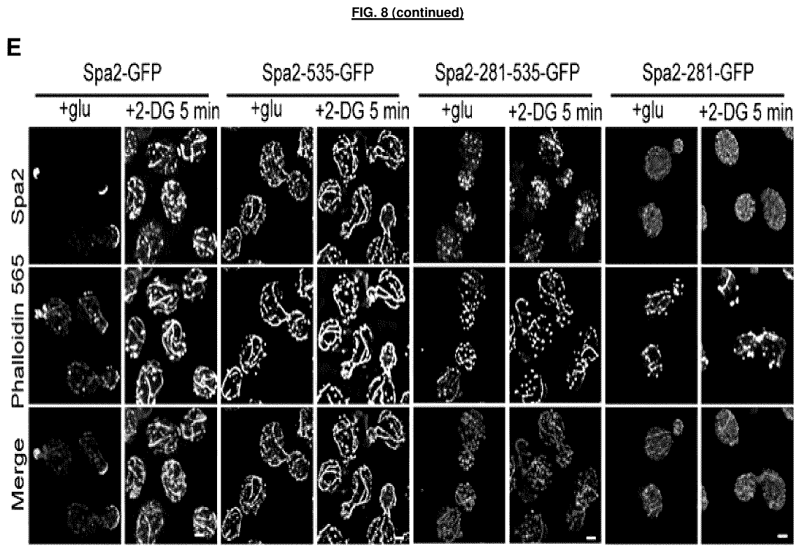

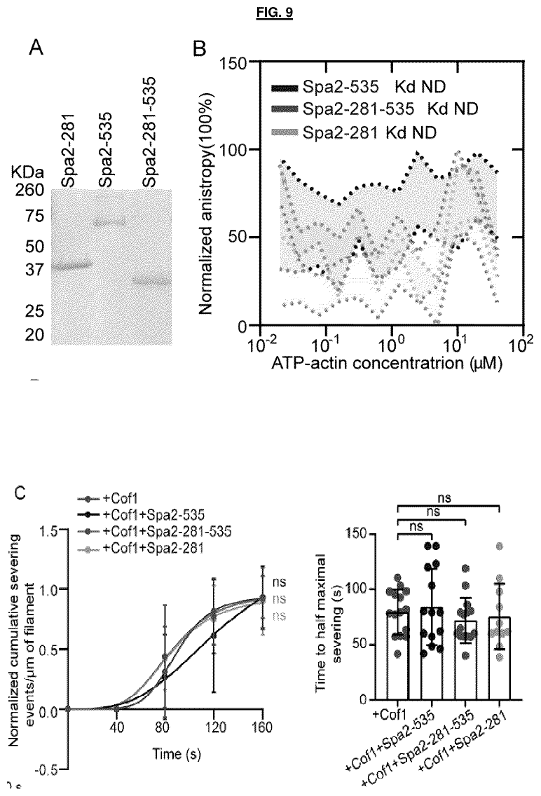

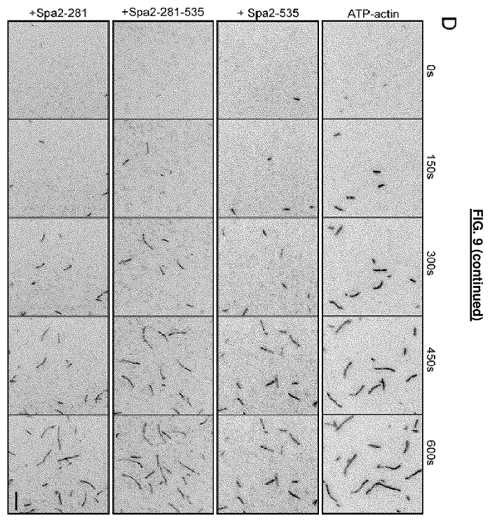

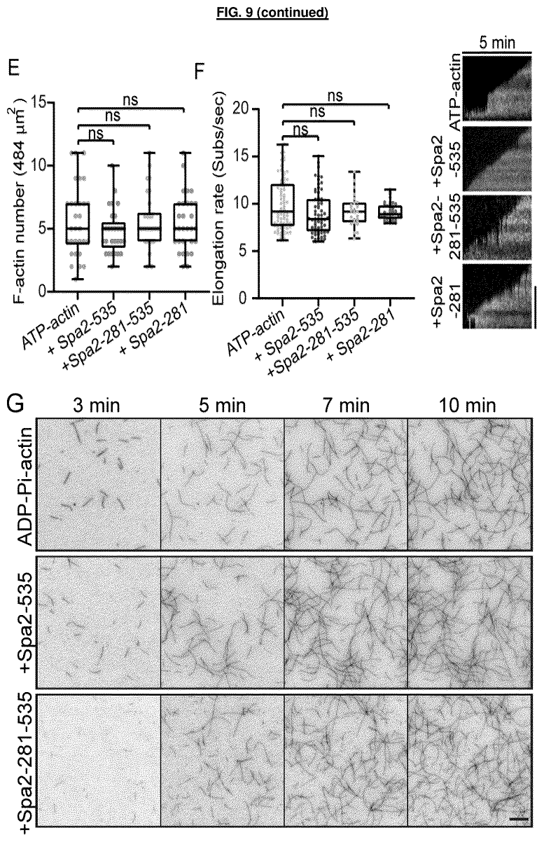

The inventors then dissected the core regions of Spa2 that are responsible for ES-triggered actin cable remodelling. Based on the prediction of structured and intrinsically disordered region (IDR) using IUPred2A (Meszaros et al., Nucleic Acids Res 46, W329-W337 (2018)), the inventors created GFP-tagged Spa2 variants with truncation at the C-terminus. In vivo localization experiments showed that Spa2-535 was the shortest examined version that retained ES-triggered filament formation (FIGS. 2A to 2C; FIG. 8A). Spa2-535 started to display a weak filamentous pattern on its own under normal growth conditions (FIG. 2B). In contrast, Spa2-281-535-GFP and Spa2-281-GFP showed cytoplasmic puncta and diffusive patterns, respectively, where neither of them can form cables (FIG. 2B). However, spa2-535 cells, but not spa2-281-535 and spa2-281 cells, maintained the ability to crosslink and stabilize actin cables by ES (FIGS. 2E to 2F). However, spa2-281-535, but not spa2-281, could still increase actin cable production upon ES (FIG. 2G; FIG. 8E), indicating that Spa2-281-535 functions in cable polymerization but not F-actin crosslinking. In addition, Spa2-535-GFP filaments also showed ATP: ADP ratio-dependent bundling upon GS in the WT background and cbp2Δ, similar to the thickening of Spa2-GFP and Abp140-3×GFP cables, but showed little response in hxk2Δ (FIGS. 8B to 8D).

### Example 3: Spa2 is an ADP-Specific Actin Nucleator that Functions Through Spa2-281-535

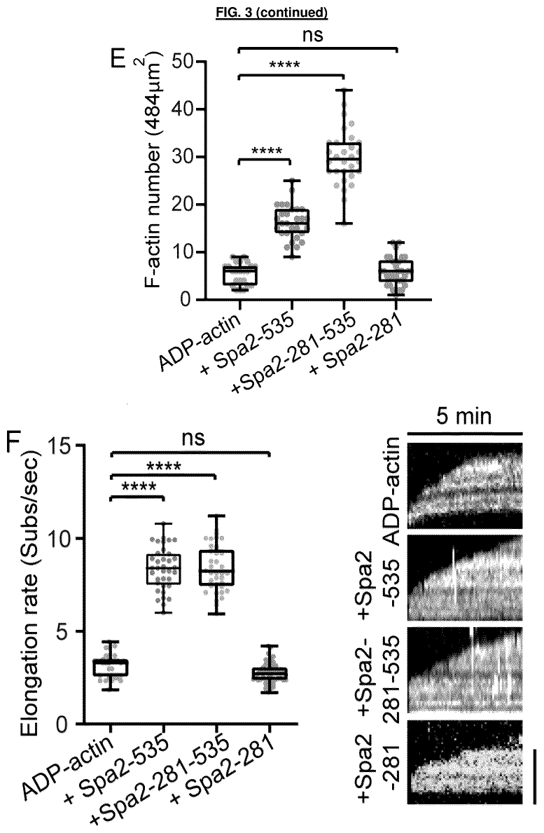

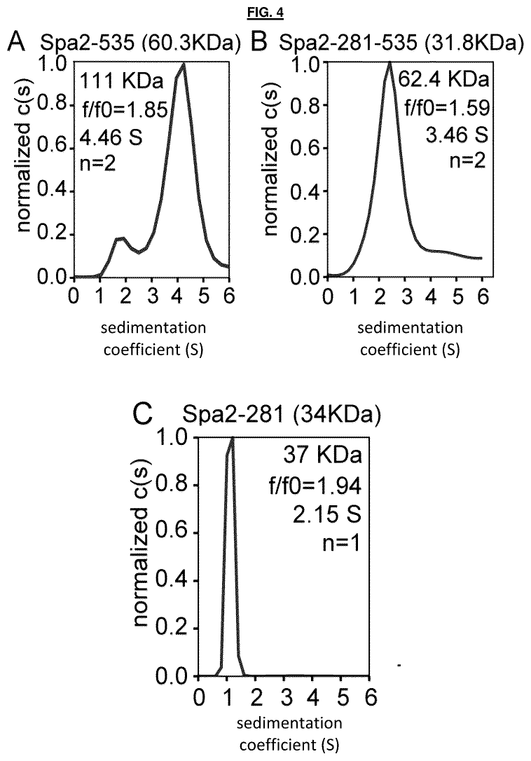

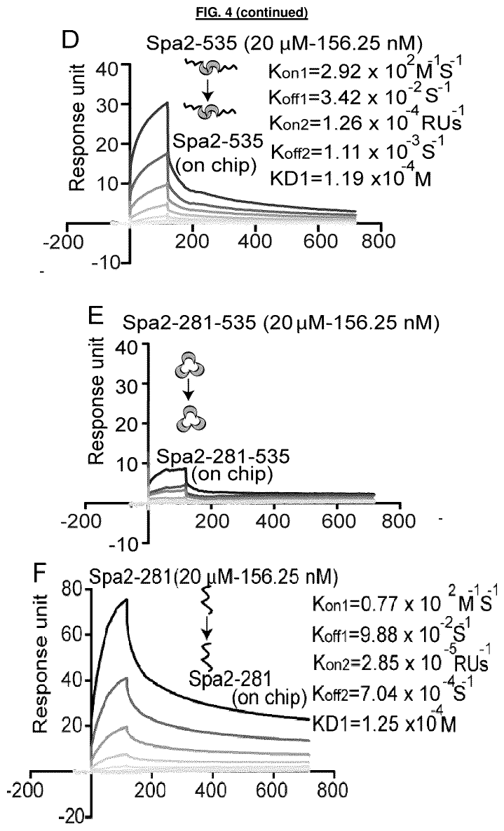

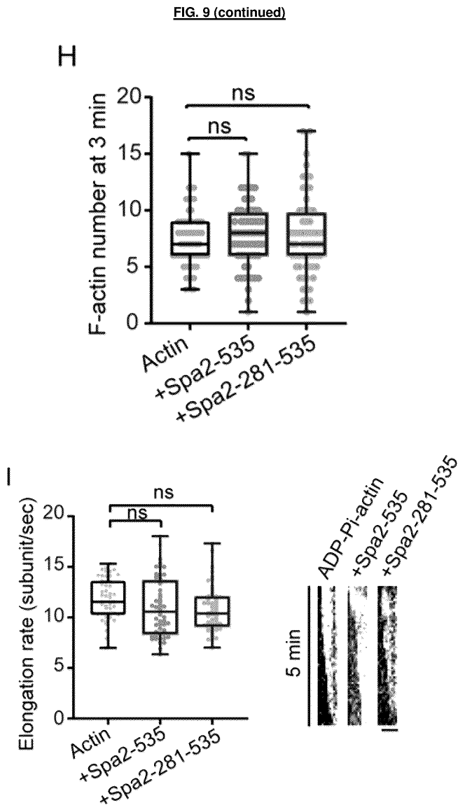

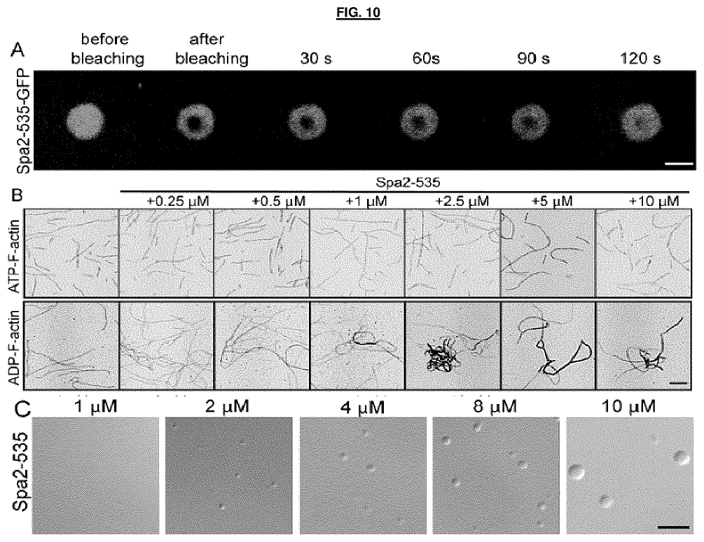

The inventors could not find a homologous region of known actin nucleators in Spa2-535, including the Arp2/3 complex, formins, or WH2-domain family proteins. To understand the Spa2-535-mediated increase in actin polymerization, the potential interactions between Spa2-535 and G-actin in both ATP- and ADP-bound states were first examined. Recombinant Spa2-535, Spa2-281-535, and Spa2-281 proteins expressed and purified from the bacteria (FIG. 9A) were mixed with ADP-G-actin or ATP-G-actin, followed by binding affinity measurement using a fluorescence anisotropy assay. Spa2-535 and Spa2-281-535 showed a similar affinity toward ADP-G-actin at Kd values of 0.36±0.11 μM and 0.24±0.04 μM, respectively, whereas no ATP-G-actin binding was detected (FIG. 3A; FIG. 9B). Spa2-281 bound neither ADP-G-actin nor ATP-G-actin (FIG. 3A; FIG. 9B). Actin nucleation and elongation activities were next examined via a total internal reflection fluorescence microscopy (TIRFM)-actin polymerization assay. To mimic the in vivo actin remodelling of pre-existing F-actin upon ES, 50 nM Cof1 and Spa2 variants were applied to pre-formed F-actin from ATP-G-actin. When actin filaments were severed quickly by Cof1, the inventors observed striking actin nucleation events after ~80 s from the samples containing either the additional Spa2-535 or Spa2-281-535, but not Spa2-281, in which a large number of short actin filaments were produced (FIGS. 3B and 3C). However, the inventors did not observe noticeable changes in Cof1's severing activities in the presence of additional Spa2-535 or Spa2-281-535 (FIG. 9C). This finding indicates de novo actin nucleation in cooperation with Cof1-mediated F-actin depolymerization.

The striking formation of F-actin during depolymerization motivated the inventors to investigate whether Spa2 can nucleate ADP-bound G-actin directly using the TIRFM-actin assay. As a result, it was observed that both Spa2-281-535 and Spa2-535, but not Spa2-281, are highly potent in nucleating ADP-G-actin by rapidly increasing seeds at a low concentration of 10 nM (FIG. 3D). The ADP-actin nucleation rate was increased by ~2.9-fold with Spa2-281-535 and ~5.4-fold with Spa2-535 (FIG. 3E). In addition, the elongation rate was also boosted by ~2.8-fold by either Spa2-281-535 or Spa2-535 (FIG. 3F). In contrast, at the same concentration, neither Spa2-281-535 nor Spa2-535 could promote the polymerization of ATP-G-actin (FIGS. 9D to 9F) or ADP-Pi-actin monomers (FIGS. 9G to 91). The results indicate that Spa2 nucleates and elongates monomeric actin in an ADP-specific manner by the N-terminus, where the region from 281 aa to 535 aa is critical.

### Example 4: Spa2 Undergoes Multivalent Self-Assembly Through the N-Terminal IDR

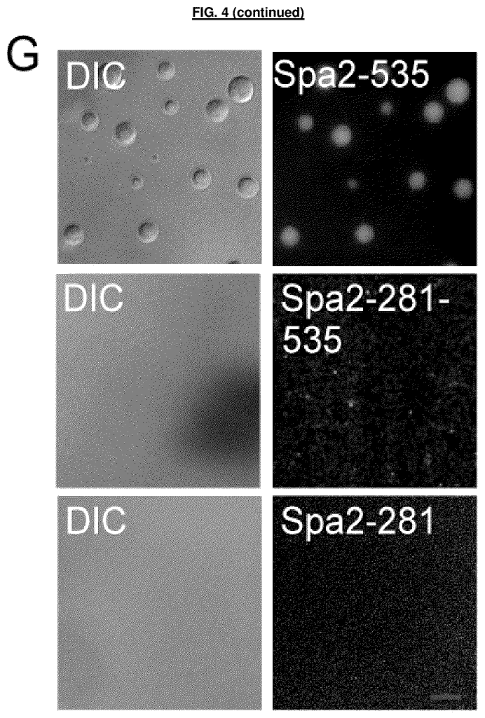

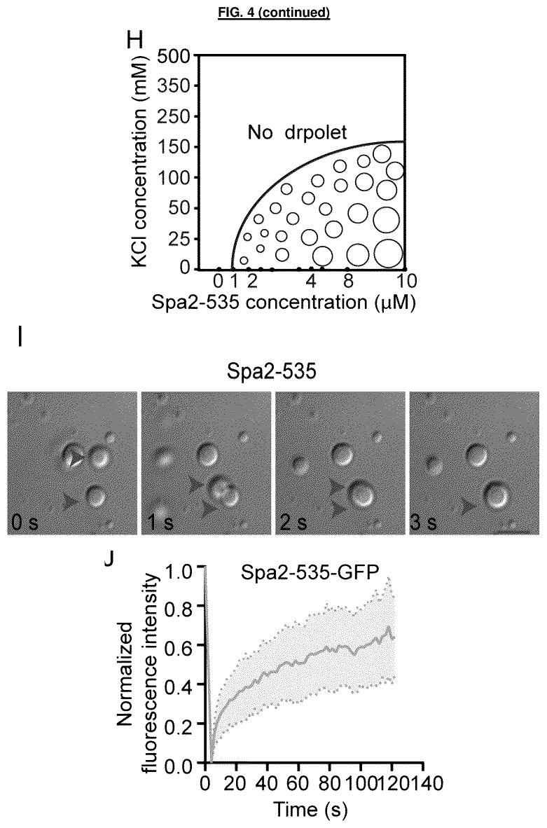

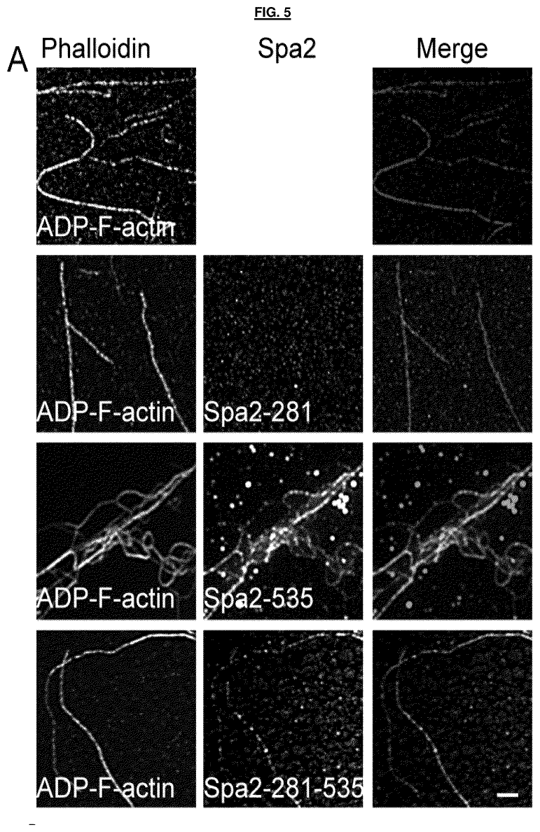

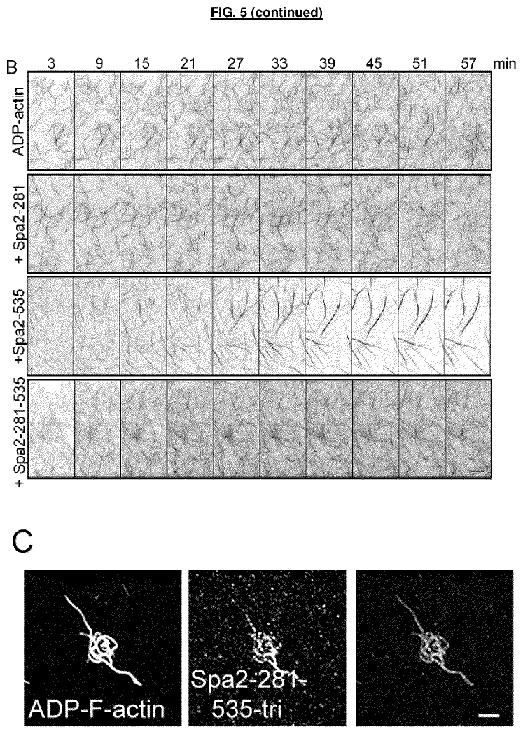

The formation of the nucleus with more than two actin subunits can surpass the rate-limiting step of actin polymerization (Pollard, T. D., and Borisy, G. G., Cell 112, 453-465 (2003)). Multivalent binding of G-actin via protein oligomerization has been known to promote nucleation (Quinlan, M. E, et al., Nature 433, 382-388 (2005)) or increase the elongation rate by incorporating multiple G-actin at the growing end (Benanti, E. L. et al., Cell 161, 348-360. (2015); Brühmann, S. et al., Proceedings of the National Academy of Sciences 114 (2017)). Here, the inventors investigated if Spa2 N-terminal domains are in a low oligomeric state, enabling active nucleation and elongation of ADP-actin. First, quantitative oligomerization measurements were performed using an analytical analysis ultracentrifugation sedimentation velocity (AUC-SV) experiment. The measurements of the sedimentation peak with three examined Spa2 variants showed dimeric states for both Spa2-535 (SEQ ID NO: 1; MWₐₚₚ=104 kDa) and Spa2-281-535 (SEQ ID NO: 2; MWₐₚₚ=93.2 kDa) but a monomer for Spa2-281 (SEQ ID NO: 5; MWₐₚₚ=37 kDa) (FIGS. 4A to 4C). While a trimerized Spa2-281-535 seems to support the ADP-G-actin nucleation well and the ~3-fold increase in elongation rate, a better understanding of the nucleation and elongation activity of Spa2-535 dimers is needed (FIGS. 3F and 3G). To understand the interaction modes in the self-association of Spa2 variants underlying their biochemical activities, their intermolecular interactions were investigated via surface plasmon resonance (SPR). Spa2-535 displayed weak inter-dimer interactions, which is likely derived from the self-interactions between the IDR (Spa2-281; SEQ ID NO: 5). Spa2-281, but not Spa2-281-535 (SEQ ID NO: 2), showed obvious self-association (FIGS. 4D to 4F)

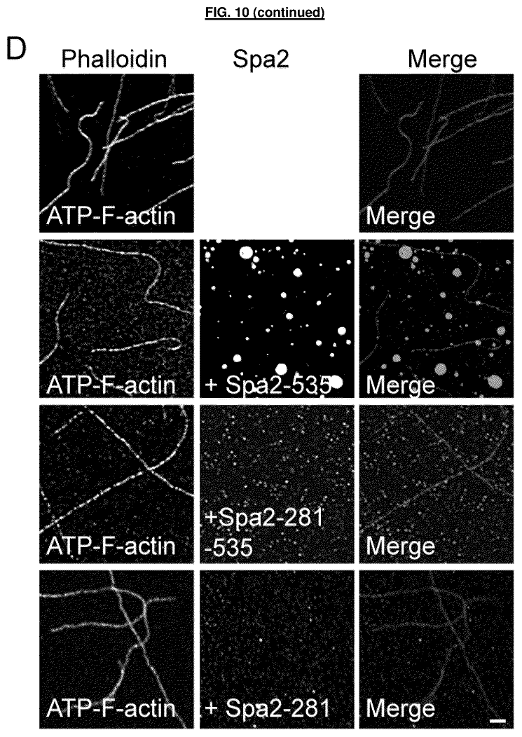

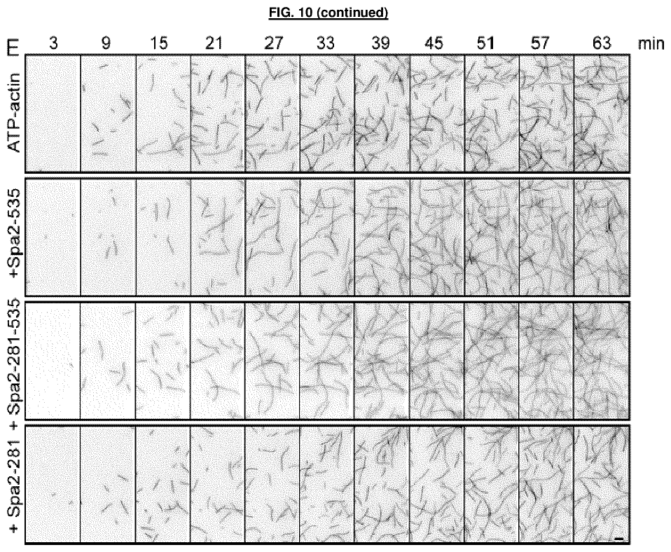

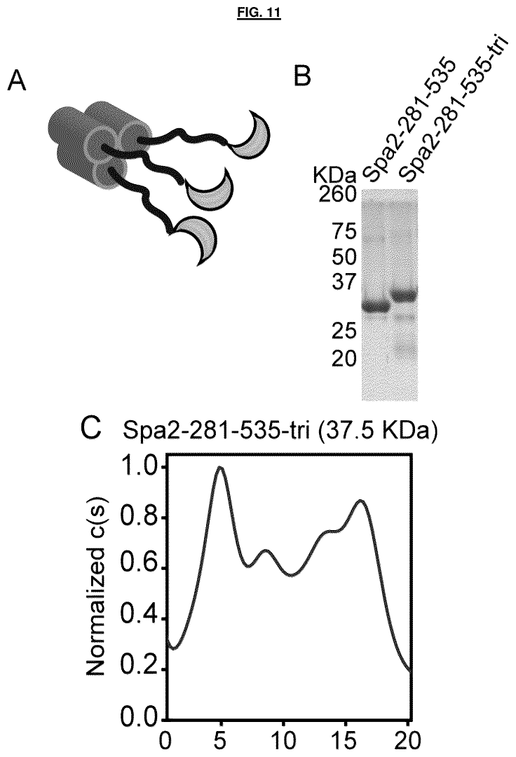

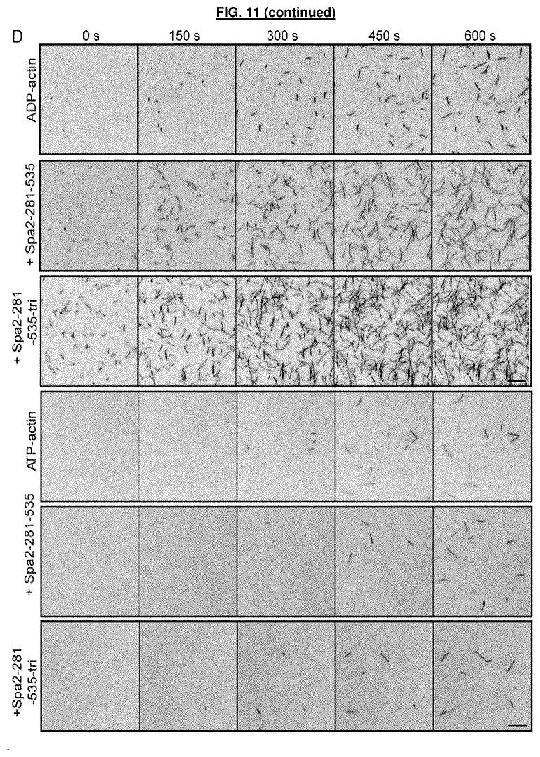

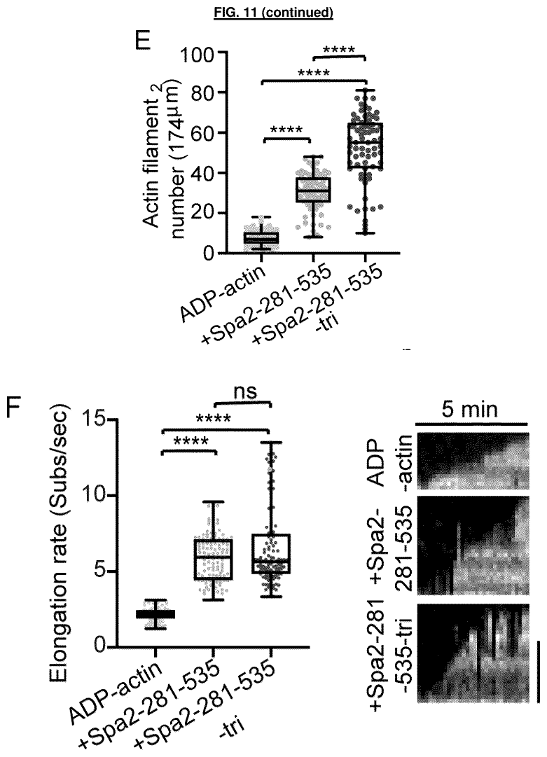

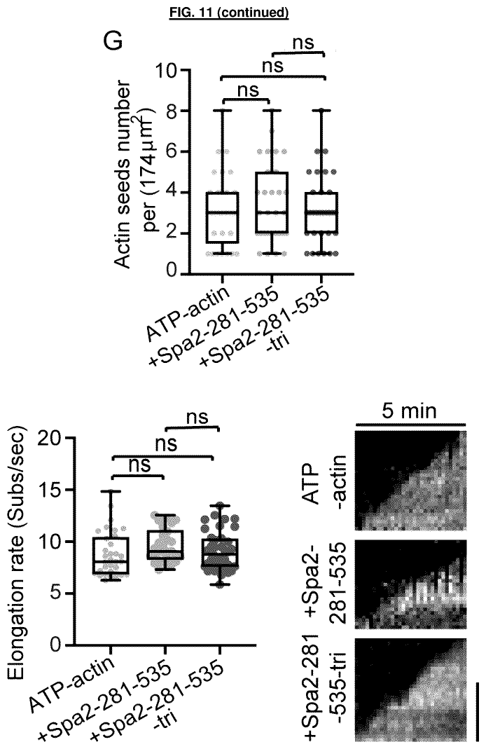

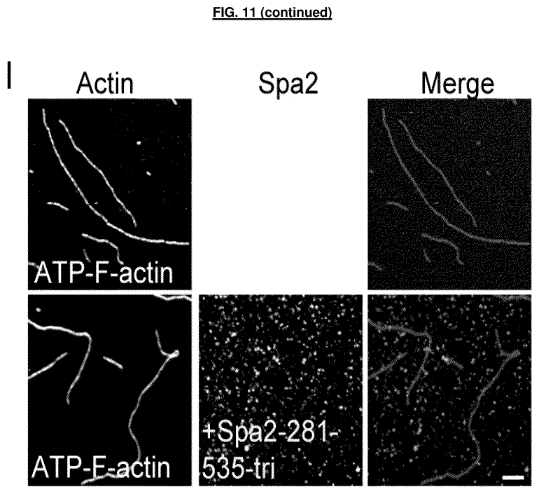

Multivalent interactions of IDR-containing proteins (IDP) have been found to promote cytoskeleton nucleation by undergoing phase separation (Sun, H. et al., Nature comms 12, 4064 (2021); King, M. R., and Petry, S., Nature comms 11, 270 (2020); Case, L. B. et al., Science 363 (2019)). Given the inter- and intra-molecular interactions shown above for Spa2-281, it was then investigated whether IDR (Spa2-281; SEQ ID NO: 5) aided in the multivalent assembly of Spa2-535 dimers by undergoing liquid-liquid phase separation (LLPS) (Choi, J. M. et al., Annu Rev Biophys 49, 107-133 (2020)). Via microscopic imaging, it was found that Spa2-535 proteins are immiscible with aqueous solvents and exhibit protein concentration- and ionic strength-dependent macromolecular condensation by forming spherical droplets (FIGS. 4G and 4H). The homotypic assembly of Spa2-535 displayed LLPS properties with coalescence (FIG. 4I) and high fluidity with rapid recovery after fluorescence photo-bleaching (FIG. 4J; FIG. 10A).

### Example 5: Spa2 Proteins Undergo Multivalent Self-Assembly Through the N-Terminal IDR

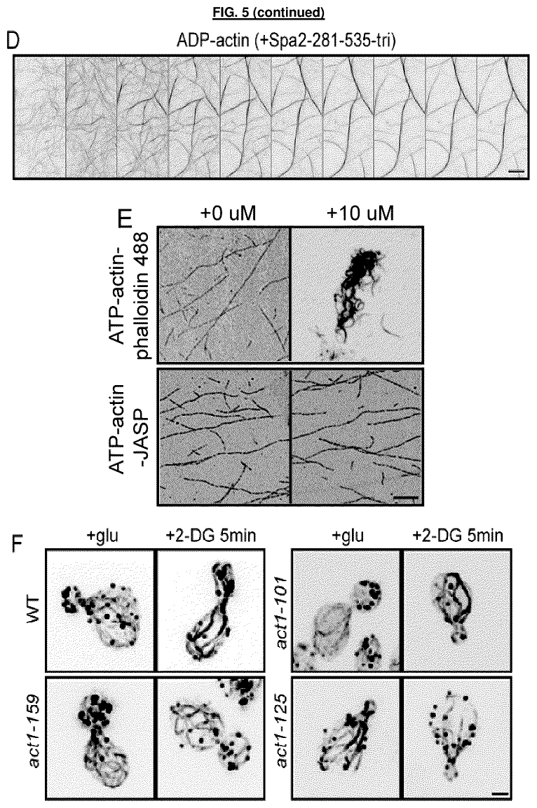

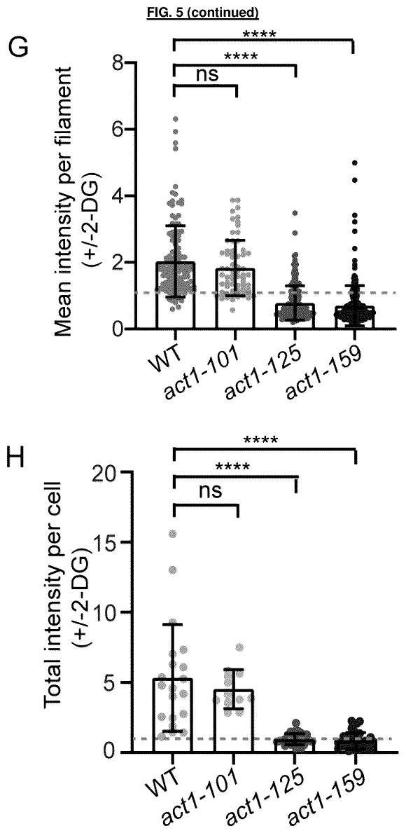

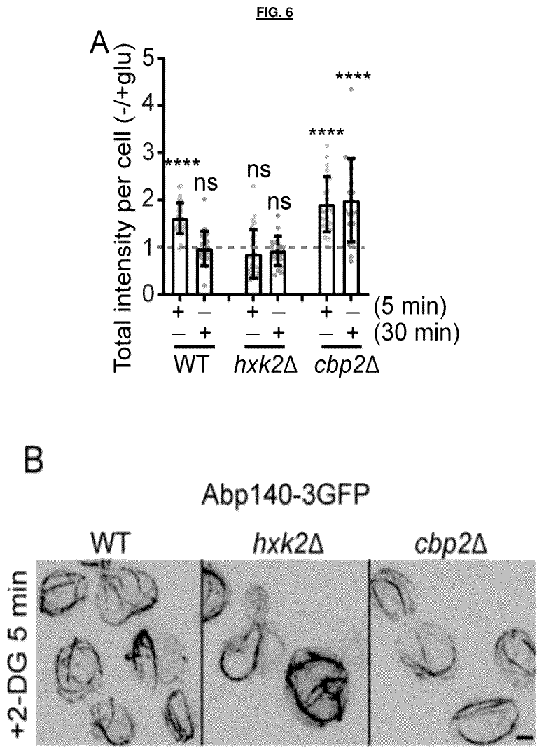

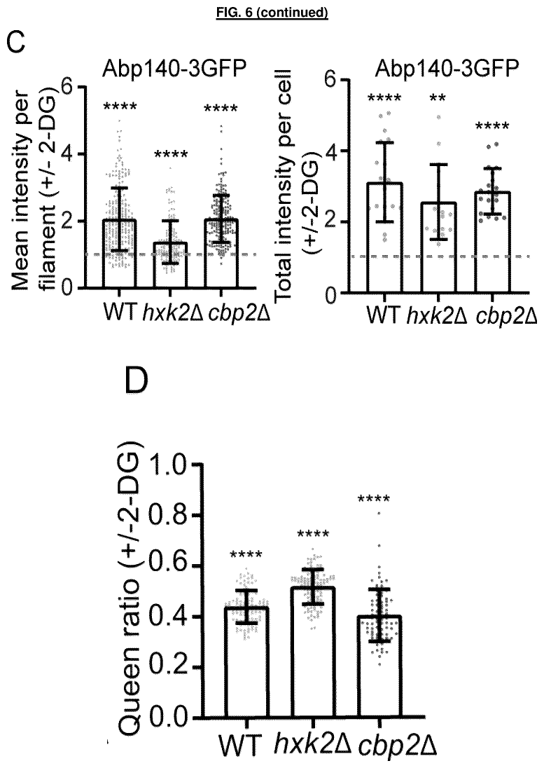

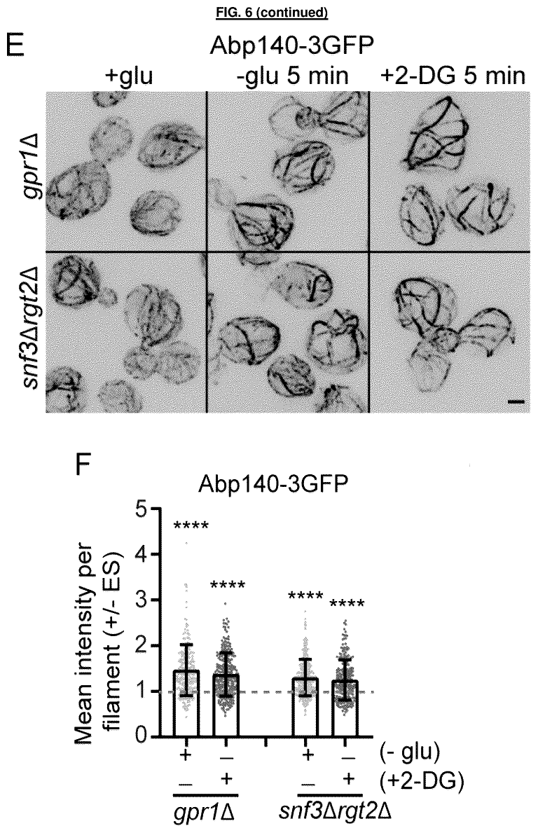

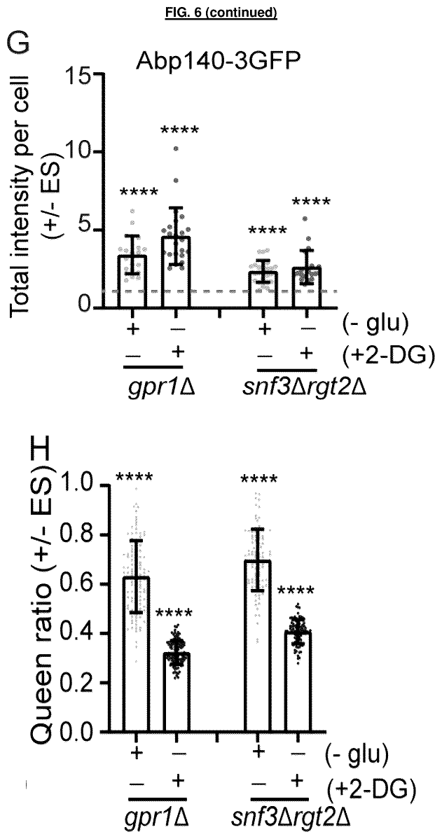

The wetting of the microtubule by the phase-separated targeting protein for Xklp2 (TPX2) results in TPX2-tubulin co-condensation (Setru, S. U. et al., Nat Phys 17, 493-498 (2021)). The dynamic multivalent Spa2-535 interactions motivated the inventors to find out whether the Spa2-535 droplets would coat F-actin in an ADP-specific manner. ADP-F-actin and ATP-F-actin filaments were incubated with 5 μM Spa2-281, Spa2-281-535, or Spa2-535. None of the Spa2 variants associated with or changed ATP-F-actin (FIG. 10D). In contrast, Spa2-281-535 proteins were decorated along filamentous ADF-F-actin as puncta, whereas Spa2-535 proteins condensed on ADP-F-actin that associated and cross-linked actin filaments (FIG. 5A). Spa2-535 mediated actin crosslinking in a concentration-dependent manner with a high correlation to Spa2 homotypic phase separation behaviour (FIG. 10B), suggesting Spa2 valency-dependent F-actin bundling.

Next, the interplay between Spa2 activities in nucleating and crosslinking in ADP-actin upon ES was investigated. Real-time actin polymerization from ADP-G-actin were monitored under TIRM over time in the presence of 5 μM Spa2-281, Spa2-281-535, and Spa2-535. Spa2-281-535 and Spa2-535 rapidly generated a large amount of ADP-actin filaments with initial nucleation and elongation within 3 min. After ~15 min in the presence of Spa2-535, F-actin filaments started to exhibit striking rearrangement, converging and bundling with a progressive increase in filament intensity over time (FIG. 5B). Interestingly, Spa2-535-induced F-actin bundles did not develop into pearling and spindle-shaped anisotropic F-actin droplets, which can be generated by actin crosslinkers, such as mammalian filamin (Weirich, K. L. et al., Proc Natl Acad Sci USA 114, 2131-2136 (2017)) and phase-separated bacterial effector XopR (Sun, H. et al., Nature comms 12, 4064 (2021)). The results indicate that Spa2-535 induces F-actin cohesion and relaxes to an elongated shape without creating isotropic interfacial tension to contract the bundles. In contrast, the examined Spa2 variant neither co-localized with ATP-F-actin filaments nor rearranged the actin network (FIGS. 10D and 10E).

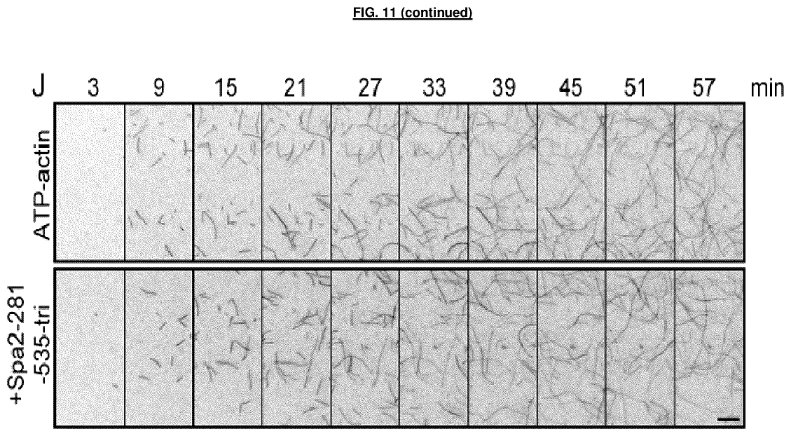

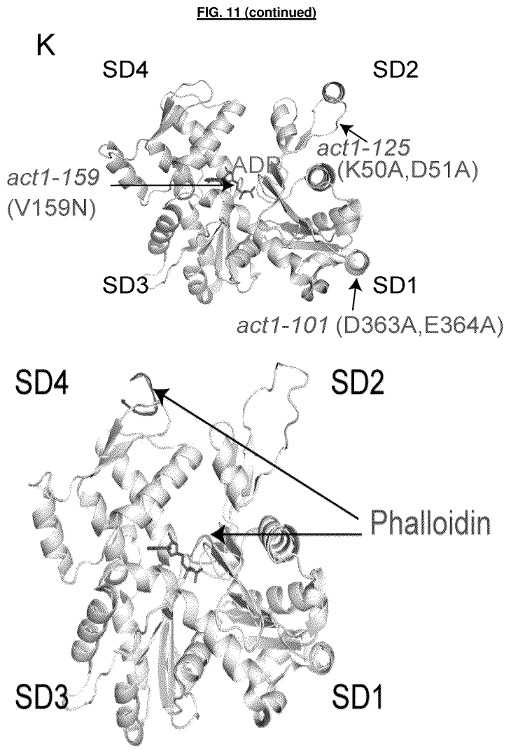

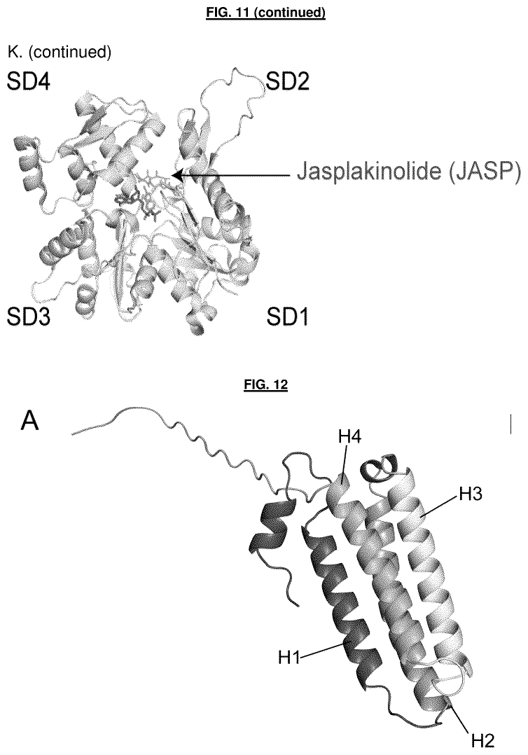

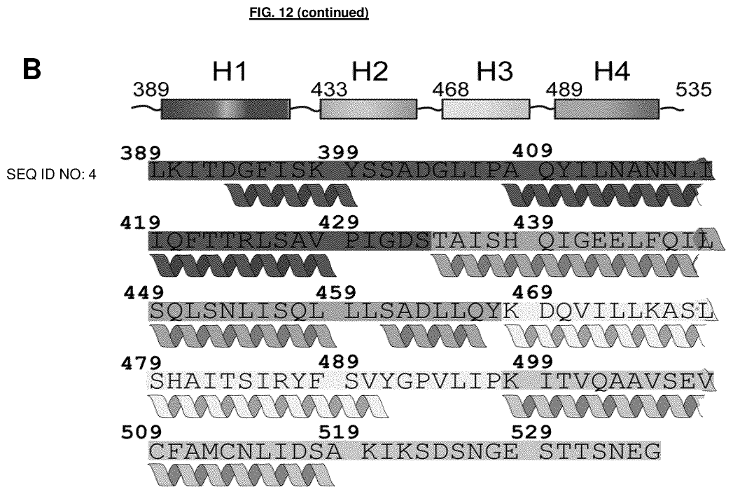

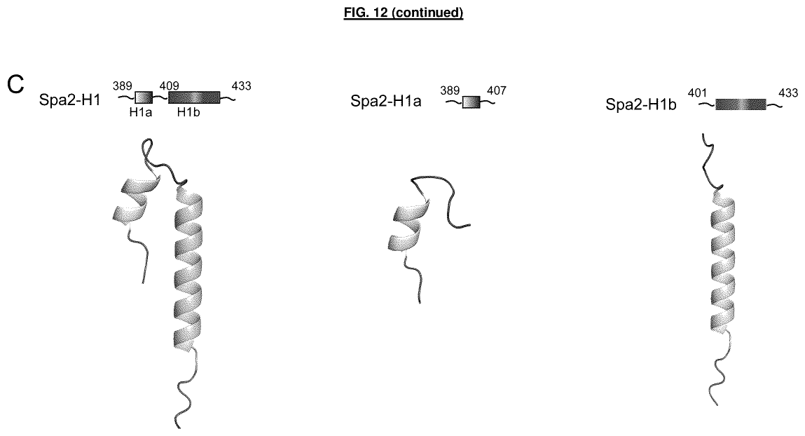

To determine the importance of Spa2 valency for actin nucleation and crosslinking, a multivalent Spa2-281-535 was created via protein engineering using a trimerization coiled-coil (CC) motif (Khairil Anuar, I. N. A. et al., Nature comms 10, 1734 (2019)) (Spa2-281-535-trimer, FIG. 11A). The produced recombinant Spa2-281-535 trimer showed higher-order oligomers than the CC-defined trimeric state (FIGS. 11B and 11C), likely due to mismatched intermolecular interactions in the combination of the trimeric CC motif and Spa2-281-535 regions. Nevertheless, this recombinant Spa2-281-535 trimer showed potent ADP-G-actin-specific nucleation activity, which was even higher than that of trimeric Spa2-281-535 (FIGS. 11 D to 11H). In addition, Spa2-281-535 trimers are also capable of crosslinking ADP-F-actin with a slightly faster F-actin convergence than Spa2-535 over time at the same protein concentration (FIG. 5B), suggesting that the efficacy in crosslinking F-actin is dependent on the oligomerization degree. In contrast, no ATP-actin-dependent activities were detected for Spa2-281-535-trimer, such as nucleation (FIGS. 11D, 11G and 11H), binding (FIG. 11I), or bundling (FIG. 11J).

### Example 6: Spa2 Interacts Preferentially with the ADP-Nucleotide Bound State Depending on the Actin D-Loop Conformation

Next, it was investigated how Spa2 differentiates actin nucleotide states. Phosphate release from ADP-pi-actin to ADP-actin turns the DNase I binding loop (D-loop) into a closed conformation, an important difference between ADP- and ADP-actin filaments. Whereas phalloidin stabilizes the D-loop in a closed conformation, jasplakinolide (JASP) inhibits phosphate release, prevents dynamic conformational changes of the intrastrand interface, and locks F-actin in the open-D-loop state regardless of the nature of bound nucleotides (Merino, F. et al., Nature structural & molecular biology 25, 528-537 (2018)) (FIG. 11K). It was found that JASP-stabilized actin abolished Spa2-535 function in crosslinking ADP-F-actin, whereas phalloidin did not (FIG. 5C). Additionally, ES-triggered actin cable remodelling was examined in different temperature-sensitive mutant alleles of ACT1 that are relevant to nucleotide exchange and the D-loop (FIG. 11K). Act1-159 (V159N) and act1-125 (K50A, D51A), which have defects in nucleotide exchange and the D-loop, displayed clear attenuation in actin cable stabilization upon ES triggering, whereas act1-101 (D363A, E364A), with a mutation in actin subdomain I, was comparable to that in WT cells (FIGS. 5D to 5F).

### Example 7: Rational Design of an ADP-Actin Specific Binding Motif Based on Structural Predictions

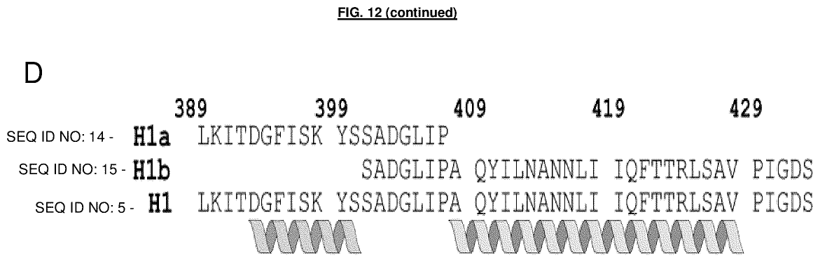

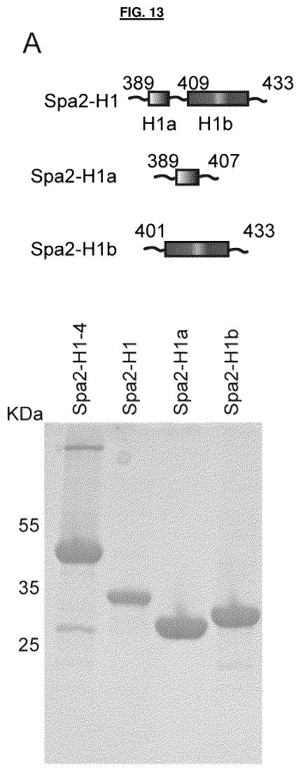

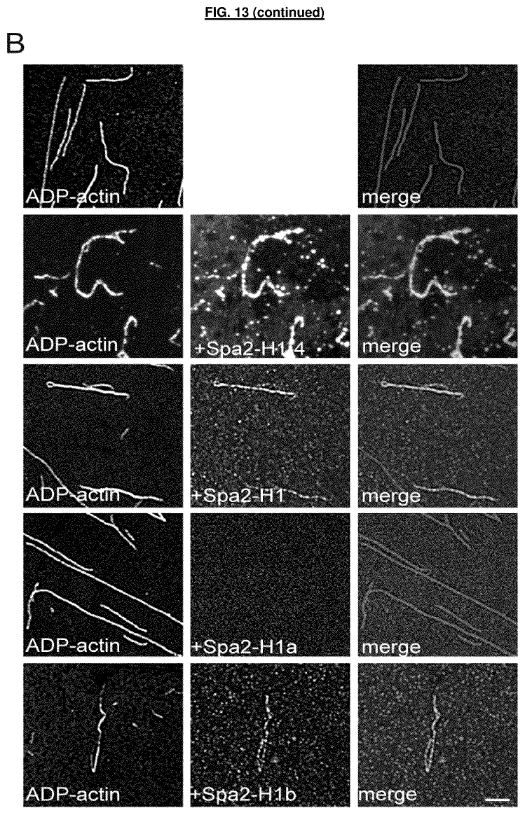

Next, to determine the specific binding site of Spa2 helix region Spa2-389-535 to ADP-actin, Spa2 structures were predicted by using Alphafold2 Software (FIGS. 12A and 12B). The Spa2 H1 domain spanning residues 389-433 was further subdivided into Spa2-H1a (residues 389-407) and Spa2-H1b (residues 401-433) (FIGS. 12C and 12D).

### Example 8: Identification and Characterization of an ADP-Actin Sensing Peptide Using msfGFP-Spa2-H1b Reporter

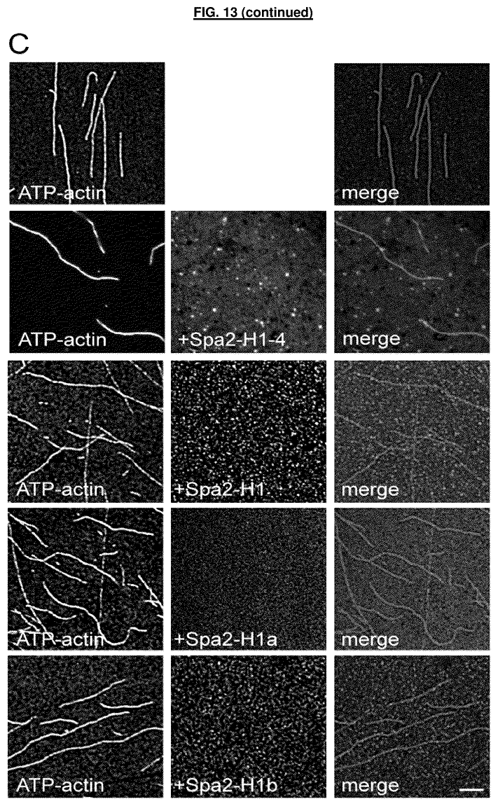

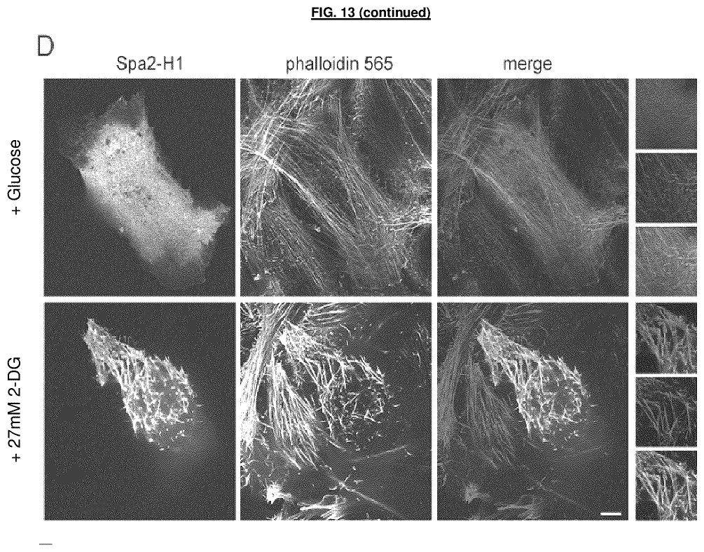

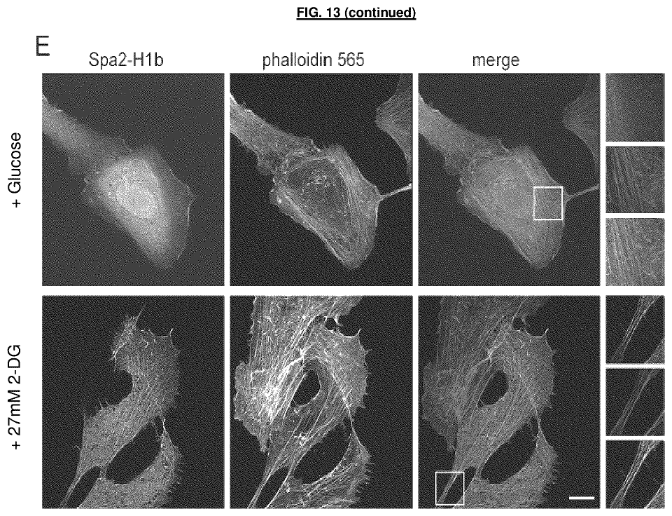

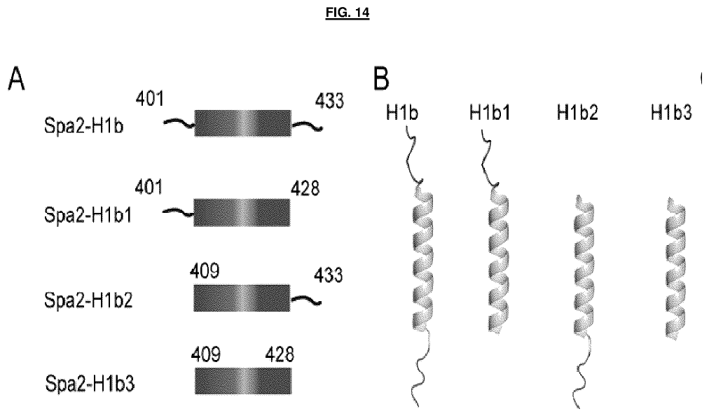

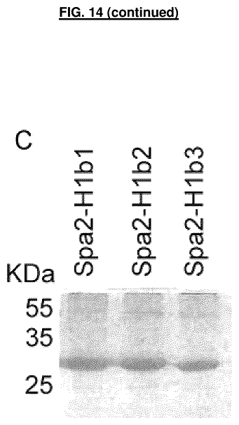

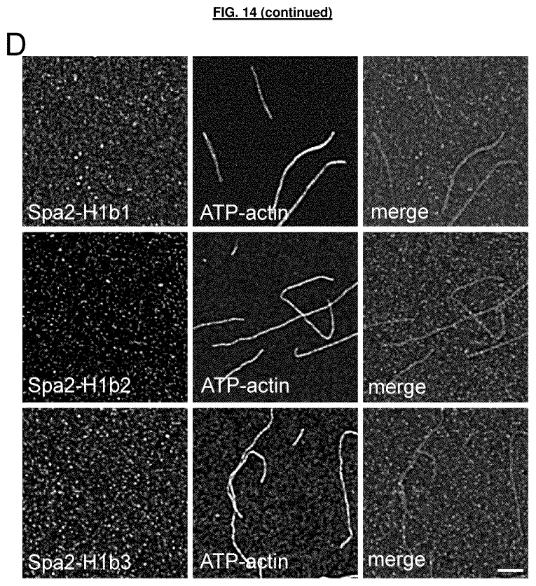

To investigate the specific binding helix of Spa2 to ADP-actin, msfGFP was tagged into different variant Spa2 truncations (FIG. 13A), and were incubated with ADP-F-actin and ATP-F-actin. It is striking that Spa2-H1 and Spa2-H1b can specifically bind to ADP-F-actin but not ATP-F-actin (FIGS. 13B, 13C). Next, GFP tagged Spa2 helix was expressed in U2OS cell and energy starvation was performed, Spa2-H1-GFP and Spa2-H1b-GFP can colocalize with ADP-F-actin.

### Example 9: Identification and Characterization of the Minimal Helix Required for ADP-Actin Sensing In Vitro and In Vivo

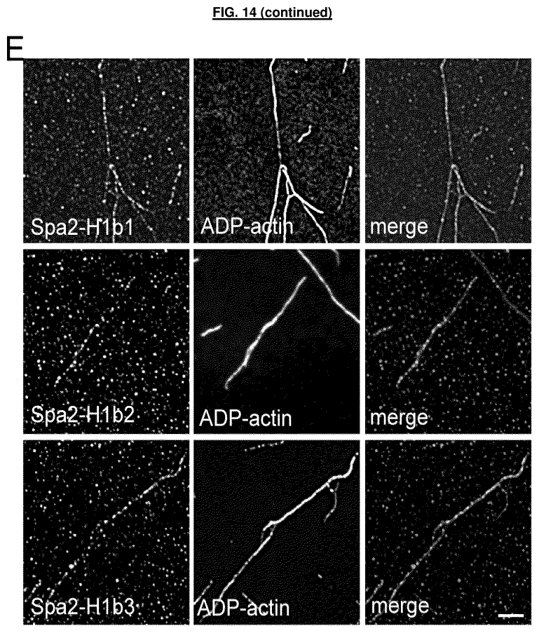

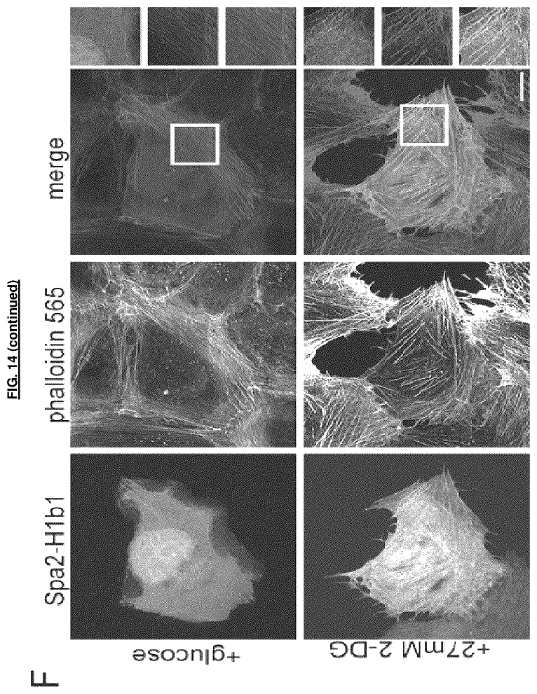

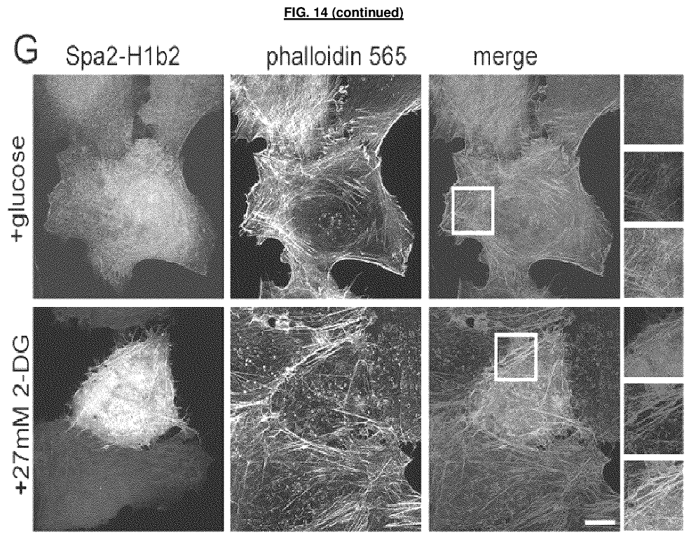

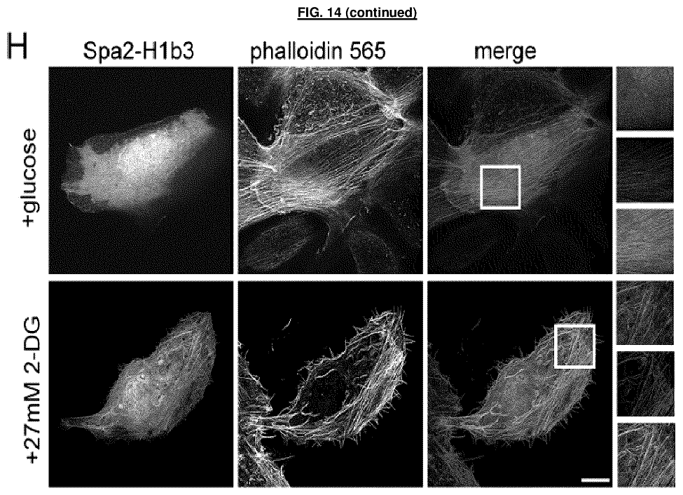

Next, to further dissect the minimum peptide of Spa2 that binding to ADP-actin, based on Alphafold prediction, Spa2-H1b was truncated into Spa2-H1b1, Spa2-H1b2 and Spa2-H1b3 (FIGS. 14A, 14B, 14C). First, in vitro actin binding assay was performed, and the data show that all of Spa2-H1b1, Spa2-H1b2 and Spa2-H1b3 can colocalize with ADP-F-actin but not ATP-F-actin (FIGS. 14D, 14E). To determine how Spa2 helix sensing ADP-actin in vivo, GFP tagged Spa2 helix was expressed in U2OS cell and energy starvation was performed. Striking, Spa2-H1b3 was the minimum domain for ADP-F-actin binding after energy starvation (FIG. 14F-14H).

### SUMMARY

The present disclosure relates to the application of SPA2-derived recombinant polypeptides in a novel mechanism of actin filament regulation under energy starvation conditions. Actin polymerization, a crucial cellular process typically driven by ATP, involves the transformation of globular actin (G-actin) into filamentous actin (F-actin). This transformation underpins various cellular activities, including cell motility, division, and intracellular transport. The process involves ATP-actin monomers attaching to the growing end of the filament, which over time, hydrolyze to ADP. The ADP-actin subunits then dissociate from the filament's other end, maintaining a dynamic equilibrium known as treadmilling.

The present invention introduces a significant departure from the traditional ATP-based actin polymerization process. The key features of the invention include, inter alia:
1. The unique capability of the recombinant polypeptides disclosed herein to control actin remodelling by initiating G-actin nucleation and triggering F-actin crosslinking in an ADP-actin-specific manner. This nucleotide-specific recognition of ADP-actin by the polypeptides of the invention open up new avenues for the development of diagnostic methods and biomarkers for adverse stress and illnesses in living organisms.
2. The high specificity of the recombinant polypeptides disclosed herein towards ADP-actin over ATP-actin, enabling timely actin remodelling under rapid energy starvation stress. This rapid response and specific recognition advantageously allow for the application of the present invention as ADP-actin sensors, and its use in multifunctional detection and potential drug molecular targeting.

The polypeptides of the present invention may also suitably be adapted for use as biochemical reagents for ADP-actin assays, in sensing technology for ADP-actin-remodelling-related human diseases, and other biotechnological applications. For example, the present invention may be adapted to develop new diagnostic technologies for diseases that alter actin cytoskeleton polymerization and network formation. The present invention could also be adapted for use in the biotech industry to engineer relevant recombinant proteins, organisms, or cells with specific sensitivity to environmental changes, such as energy starvation conditions.

### REFERENCES

Ali, R., Zahm, J. A., and Rosen, M. K. (2022). Bound nucleotide can control the dynamic architecture of monomeric actin. Nature structural & molecular biology 29, 320-328.
Atkinson, S. J., Hosford, M. A., and Molitoris, B. A. (2004). Mechanism of actin polymerization in cellular ATP depletion. J Biol Chem 279, 5194-5199.
Benanti, E. L., Nguyen, C. M., and Welch, M. D. (2015). Virulent Burkholderia species mimic host actin polymerases to drive actin-based motility. Cell 161, 348-360.
Brühmann, S., et al., (2017). Distinct VASP tetramers synergize in the processive elongation of individual actin filaments from clustered arrays. Proceedings of the National Academy of Sciences 114, E5815-E5824.
Case, L. B., et al., (2019). Stoichiometry controls activity of phase-separated clusters of actin signaling proteins. Science 363, 1093-1097.
Choi, J. M., Holehouse, A. S., and Pappu, R. V. (2020). Physical Principles Underlying the Complex Biology of Intracellular Phase Transitions. Annu Rev Biophys 49, 107-133.
Garabedian M. V., et al., (2018) Integrated control of formin-mediated actin assembly by a stationary inhibitor and a mobile activator J Cell Biol. 217 (10), 3512-3530.
Jumper J. et al. (2021) Highly accurate protein structure prediction with AlphaFold. Nature 596, pages 583-589.
Khairil Anuar, I. N. A., et al., (2019). Spy&Go purification of SpyTag-proteins using pseudo-SpyCatcher to access an oligomerization toolbox. Nature communications 10, 1734.
King, M. R., and Petry, S. (2020). Phase separation of TPX2 enhances and spatially coordinates microtubule nucleation. Nature communications 11, 270.
Kudryashov, D. S., et al., (2010). A nucleotide state-sensing region on actin. J Biol Chem 285, 25591-25601.
Longtine, M. S., et al., (1998) Additional modules for versatile and economical PCR-based gene deletion and modification in Saccharomyces cerevisiae. Yeast 14 (10); 953-961.
Merino, F., et al., (2018). Structural transitions of F-actin upon ATP hydrolysis at near-atomic resolution revealed by cryo-EM. Nature structural & molecular biology 25, 528-537.
Meszaros, B., Erdos, G., and Dosztanyi, Z. (2018). IUPred2A: context-dependent prediction of protein disorder as a function of redox state and protein binding. Nucleic Acids Res 46, W329-W337.
Miao, Y., et al., (2016) Fimbrin phosphorylation by metaphase Cdk1 regulates actin cable dynamics in budding yeast. Nature Communications 7, 11265.
Pollard, T. D., and Borisy, G. G. (2003). Cellular motility driven by assembly and disassembly of actin filaments. Cell 112, 453-465.
Pollard, T. D., and Cooper, J. A. (2009). Actin, a central player in cell shape and movement. Science 326, 1208-1212.
Quinlan, M. E., et al., (2005). Drosophila Spire is an actin nucleation factor. Nature 433, 382-388.
Roth, S., and Heintzmann, R. (2016) Optical photon reassignment with increased axial resolution by structured illumination. Methods Appl. Fluoresc. 4, 045005.
Sun, H., et al., (2021). Xanthomonas effector XopR hijacks host actin cytoskeleton via complex coacervation. Nature communications 12, 4064.
Weber, C. A., et al., (2020). beta-Oxidation and autophagy are critical energy providers during acute glucose depletion in Saccharomyces cerevisiae. Proc Natl Acad Sci USA 117, 12239-12248.
Setru, S. U., et al., (2021). A hydrodynamic instability drives protein droplet formation on microtubules to nucleate branches. Nat Phys 17, 493-498.
Schindelin J., et al., (2012) Fiji: an open-source platform for biological-image analysis; Nature Methods 9, 676-682.
Tanaka K, et al., (2018). Structural basis for cofilin ‘binding and actin filament disassembly. Nat Commun. 9, 1860.
Weirich, K. L., et al., (2017). Liquid behavior of crosslinked actin bundles. Proc Natl Acad Sci USA 114, 2131-2136.
Xie, Y., and Miao, Y. (2021). Polarisome assembly mediates actin remodeling during polarized yeast and fungal growth. J Cell Sci 134, jcs.247916.
Xie, Y., et al., (2019). Polarisome scaffolder Spa2-mediated macromolecular condensation of Aip5 for actin polymerization. Nature communications 10, 1-18.
Xu, L., and Bretscher, A. (2014). Rapid glucose depletion immobilizes active myosin V on stabilized actin cables. Curr Biol 24, 2471-2479.
York A. G., et al., (2012) H. Resolution doubling in live, multicellular organisms via multifocal structured illumination microscopy. Nature Methods 9, 749-754.

1. A recombinant polypeptide comprising an amino acid sequence of at least 85% identity to the amino acid sequence set forth in SEQ ID NO: 1, or SEQ ID NO: 2, or a functional fragment or variant thereof, wherein the polypeptide, fragment or variant thereof specifically binds to ADP-actin and modulates ADP-actin remodelling.

2. The recombinant polypeptide of claim 1, wherein the recombinant polypeptide comprises or consists of an amino acid sequence of at least 85% identity to an amino acid sequence selected from the group comprising SEQ ID NO: 3, SEQ ID NO: 4, SEQ ID NO: 5 and SEQ ID NO: 6.

3. The recombinant polypeptide of claim 1, wherein the modulating of ADP-actin remodelling is selected from one or more of the group comprising enhancing ADP-actin nucleation, elongation/polymerization, crosslinking, bundling, stabilization and/or inhibition of actin depolymerization.

4. The recombinant polypeptide of claim 1, wherein the polypeptide consists of the amino acid sequence set forth in SEQ ID NO: 2, SEQ ID NO: 3, SEQ ID NO: 4, SEQ ID NO: 5 or SEQ ID NO: 6.

5. The recombinant polypeptide of claim 1, wherein the ADP-actin remodelling occurs under an energy starvation condition.

6. The recombinant polypeptide of claim 1, wherein the polypeptide comprises or consists of the amino acid sequence set forth in SEQ ID NO: 1 or SEQ ID NO: 2 and enhances ADP-actin crosslinking and/or bundling.

7. The recombinant polypeptide of claim 1, wherein the polypeptide comprises or consists of the amino acid sequence set forth in SEQ ID NO: 3, SEQ ID NO: 4, SEQ ID NO: 5 or SEQ ID NO: 6 and enhances ADP-actin nucleation and/or elongation/polymerization.

8. The recombinant polypeptide of claim 1, wherein the polypeptide is coupled to at least one heterologous molecule.

9. The recombinant polypeptide of claim 8, wherein the heterologous molecule is a fluorescent polypeptide.

10. The recombinant polypeptide of claim 1, wherein the polypeptide is multimeric.

11. A polynucleic acid molecule encoding the polypeptide of claim 1, wherein said polynucleic acid has a sequence that has at least 85% identity to the polynucleic acid sequence set forth in SEQ ID NO: 8 or in SEQ ID NO: 9, or a polypeptide-encoding fragment thereof.

12. The polynucleic acid molecule according to claim 11, wherein the polynucleic acid molecule is operatively linked to a promoter sequence.

13. A recombinant cell transfected or transformed with the polynucleic acid molecule of claim 11.

14. A method for the detection and/or modulation of ADP-actin remodelling, or in drug screening of candidate modulators of ADP-actin remodelling may comprise (i) expressing a tagged recombinant polypeptide of claim 1 in a cell; (ii) detecting whether the tagged polypeptide associates with ADP-actin or F-actin using total internal reflection (TIRF) microscopy; and for drug screening, exposing the cell before step (ii) to candidate modulators of ADP-actin remodelling.

15. The method according to claim 14, wherein the method is in vitro.

16. A pharmaceutical composition comprising a recombinant polypeptide of claim 1 and a pharmaceutically acceptable carrier.

17. A recombinant polypeptide of claim 1, a polynucleic acid molecule encoding the recombinant polypeptide, a recombinant cell transfected or transformed with the polynucleic acid molecule or a pharmaceutical composition comprising the recombinant polypeptide and a pharmaceutically acceptable carrier for use in medicine.

18. (canceled)

19. A method of treating a disease associated with actin-remodelling, comprising administering a therapeutically effective amount of a recombinant polypeptide of claim 1 or a pharmaceutical composition comprising the recombinant polypeptide and a pharmaceutically acceptable carrier, to a patient in need thereof.

20. (canceled)

21. The method of claim 19, wherein the disease associated with actin-remodelling is selected from the group comprising cancer, myopathies, familial thoracic aortic aneurysms, heart diseases, Baraitser-Winter syndrome and auto-inflammatory diseases.

22. A method, comprising screening for drugs or studying diseases associated with actin-remodelling with a recombinant polypeptide of claim 1, a polynucleic acid molecule encoding the recombinant polypeptide, or a recombinant cell transfected or transformed with the polynucleic acid molecule in a cell-based screen, wherein the method is not performed in a human.


SEQUENCE LISTING (26 sequences)

SEQ ID NO: 1 - protein, 1466 aa, Saccharomyces cerevisiae
MGTSSEVSLAHHRDIFHYYVSLKTFFEVTGENRDRSNSTRAQKARAKLLKLSSSQFYELSTDVSDELQRRIGEDANQPDYLLPKANFHMKRNQARQKLANLSQTRFNDLLDDILFEIKRRGFDKDLDAPRPPLPQPMKQEVSKDSDDTARTSTNSSSVTQVAPNVSVQPSLVIPKMASIDWSSEEEEEEQVKEKPNEPEGKQTSMDEKKEAKPALNPIVTDSDLPDSQVLARDITSMARTPTTTHKNYWDVNDSPIIKVDKDIDNEKGPEQLKSPEVQRAENNNPNSEMEDKVKELTDLNSDLHLQIEDLNAKLASLTSEKEKEKKEEKEEKEKEKNLKINYTIDESFQKELLSLNSQIGELSIENENLKQKISEFELHQKKNDNHNDLKITDGFISKYSSADGLIPAQYILNANNLIIQFTTRLSAVPIGDSTAISHQIGEELFQILSQLSNLISQLLLSADLLQYKDQVILLKASLSHAITSIRYFSVYGPVLIPKITVQAAVSEVCFAMCNLIDSAKIKSDSNGESTTSNEGNRQVLEYSSPTATTPMTPTFPSTSGINMKKGFINPRKPASFLNDVEEEESPVKPLKITQKAINSPIIRPSSSNGVPTTSRKPSGTGLFSLMIDSSIAKNSSHKEDNDKYVSPIKAVTSASNSASSNISEIPKLTLPPQAKIGTVIPPSENQVPNIKIENTEEDNKRSDITNEISVKPTSSIADKLKQFEQSSEKKSSPKENPIAKEEMDSKPKLSNKFITSMNDVSTDDSSSDGNENDDADDDDDFTYMALKQTMKREGSKIEKNNDSKLPANIVELDLHESPESVKIESPESIKEITSSEMSSEMPSSSLPKRLVEDVEPSEMPEKGASVESVRKKNFQEPLGNVESPDMTQKVKSLGMTGKAVGPESDSRVESPGMTGQIKSLNMAGKVVGPEADSRVESPGMKEQIKSLGMTGKITAQESIKSPEAARKLASSGEVDKIESPRMVRESESLEAVGNTIPSNMTVKMESPNLKGNTVSEPQEIRRDIASSEPIENVDPPKVLKKIVFPKAVNRTGSPKSVEKTPSSATLKKSGLPEPNSQIVSPELAKNSPLAPIKKNVELRETNKPHTETITSVEPTNKDANTSWRDADLNRTIKREEEDEDFDRVNHNIQITGAYTKTGKIDYHKIPVDRKAKSEAEVHTSEEDIDESNNVNGKRADAQIHITERKHAFVNPTENSQVKKTSHSPFLNSKPVQYENSESNGGINNHIKIKNTGETTAHDEKHYSDDDDSSYQFVPMKHEEQEQEQNRSEEEESEDDDEEEEDSDFDVDTFDIENPDNTLSELLLYLEHQTMDVISTIQSLLTSIKKPQVTKGNLRGESNAINQVIGQMVDATSISMEQSRNANLKKHGDWVVQSLRDCSRRMTILCQLTGDGILAKEKSDQDYADKNFKQRLAGIAFDVAKCTKELVKTVEEASLKDEINYLNSKLK

SEQ ID NO: 2 - protein, 535 aa, Saccharomyces cerevisiae
MGTSSEVSLAHHRDIFHYYVSLKTFFEVTGENRDRSNSTRAQKARAKLLKLSSSQFYELSTDVSDELQRRIGEDANQPDYLLPKANFHMKRNQARQKLANLSQTRFNDLLDDILFEIKRRGFDKDLDAPRPPLPQPMKQEVSKDSDDTARTSTNSSSVTQVAPNVSVQPSLVIPKMASIDWSSEEEEEEQVKEKPNEPEGKQTSMDEKKEAKPALNPIVTDSDLPDSQVLARDITSMARTPTTTHKNYWDVNDSPIIKVDKDIDNEKGPEQLKSPEVQRAENNNPNSEMEDKVKELTDLNSDLHLQIEDLNAKLASLTSEKEKEKKEEKEEKEKEKNLKINYTIDESFQKELLSLNSQIGELSIENENLKQKISEFELHQKKNDNHNDLKITDGFISKYSSADGLIPAQYILNANNLIIQFTTRLSAVPIGDSTAISHQIGEELFQILSQLSNLISQLLLSADLLQYKDQVILLKASLSHAITSIRYFSVYGPVLIPKITVQAAVSEVCFAMCNLIDSAKIKSDSNGESTTSNEG

SEQ ID NO: 3 - protein, 254 aa, Saccharomyces cerevisiae
NNNPNSEMEDKVKELTDLNSDLHLQIEDLNAKLASLTSEKEKEKKEEKEEKEKEKNLKINYTIDESFQKELLSLNSQIGELSIENENLKQKISEFELHQKKNDNHNDLKITDGFISKYSSADGLIPAQYILNANNLIIQFTTRLSAVPIGDSTAISHQIGEELFQILSQLSNLISQLLLSADLLQYKDQVILLKASLSHAITSIRYFSVYGPVLIPKITVQAAVSEVCFAMCNLIDSAKIKSDSNGESTTSNEG

SEQ ID NO: 4 - protein, 147 aa, Saccharomyces cerevisiae
LKITDGFISKYSSADGLIPAQYILNANNLIIQFTTRLSAVPIGDSTAISHQIGEELFQILSQLSNLISQLLLSADLLQYKDQVILLKASLSHAITSIRYFSVYGPVLIPKITVQAAVSEVCFAMCNLIDSAKIKSDSNGESTTSNEG

SEQ ID NO: 5 - protein, 45 aa, Saccharomyces cerevisiae
LKITDGFISKYSSADGLIPAQYILNANNLIIQFTTRLSAVPIGDS

SEQ ID NO: 6 - protein, 20 aa, Saccharomyces cerevisiae
QYILNANNLIIQFTTRLSAV

SEQ ID NO: 7 - protein, 281 aa, Saccharomyces cerevisiae
MGTSSEVSLAHHRDIFHYYVSLKTFFEVTGENRDRSNSTRAQKARAKLLKLSSSQFYELSTDVSDELQRRIGEDANQPDYLLPKANFHMKRNQARQKLANLSQTRFNDLLDDILFEIKRRGFDKDLDAPRPPLPQPMKQEVSKDSDDTARTSTNSSSVTQVAPNVSVQPSLVIPKMASIDWSSEEEEEEQVKEKPNEPEGKQTSMDEKKEAKPALNPIVTDSDLPDSQVLARDITSMARTPTTTHKNYWDVNDSPIIKVDKDIDNEKGPEQLKSPEVQRAE

SEQ ID NO: 8 - DNA, 4401 bp, Saccharomyces cerevisiae
  source 1..4401: mol_type = genomic DNA
atgggtacgtcaagcgaggtttctctcgcacatcatagagatatcttccattactacgtctcactgaagacttttttcgaggtgactggcgaaaatcgtgacaggtcaaattcgacacgagctcaaaaggccagagccaagctgttgaagctatcttcttcgcaattttacgagctgagtacagacgtgtccgatgagctgcagaggagaatcggtgaagatgctaaccaaccagattaccttttgccgaaggcaaatttccacatgaaaaggaaccaggctagacagaaactggccaatctatcacaaactcgatttaatgatttgttggacgatatcctttttgagatcaagagaagagggttcgacaaggatttggatgctccacggcccccattaccgcagccgatgaaacaagaggtcagcaaagacagcgatgatactgcaagaacatccacaaattcttcctctgtgactcaagtagctccaaacgtctccgtacaaccttctttggtcattcctaagatggcatctatcgattggtcttctgaggaagaagaagaggagcaagtaaaggagaagccaaatgaaccggagggaaaacaaacaagcatggatgaaaagaaagaggctaaacctgctctaaaccccatagttacagattctgatctgcctgactcccaagtgctcgctcgtgatatcacatcaatggcaaggactccaacaacgacgcataaaaattactgggacgttaatgattctccaattatcaaggtagataaagatatcgataacgaaaagggtcccgaacagttgaagagccctgaagtacaacgggctgagaacaataaccctaactcagagatggaagacaaggttaaagaactgactgatttaaacagcgacttacatttgcaaattgaagatttgaatgctaagttagcatctttaaccagcgagaaggaaaaggagaagaaggaagagaaggaggaaaaagaaaaggaaaagaacttaaagattaactacaccattgatgaaagttttcagaaagaattgctgtcattaaactctcaaatcggtgaattatcaattgagaatgaaaatttgaagcagaaaatttcagaattcgaactgcatcaaaaaaagaatgacaaccataatgatttgaaaatcactgacggttttattagcaagtactcttctgccgatgggctcattccagctcaatacatcttaaacgctaacaacttgataattcaatttactactaggctttccgcagtacccataggcgactccacggcaatttcccatcaaattggcgaagagttatttcaaatattatcccagttatcgaacctaatctcccagctattactatcggccgacctattacagtacaaagatcaggtcattttactgaaggcatcattatcgcatgcgatcacatcgataagatatttctctgtttacggtcccgtattaattccgaaaataactgtgcaagctgctgtttcagaggtttgttttgccatgtgtaatctaattgattcagcgaaaataaaatccgattcaaatggtgagagcaccacctctaatgaaggtaaccgacaggtattagaatattcttcaccaactgctaccaccccaatgacgccaactttcccctcgacttctggaataaatatgaagaaggggtttataaacccaagaaaaccagcatctttcttgaatgatgtggaggaagaagaatctccagtcaagccattgaaaattacacaaaaggcaattaacagtccgatcataagaccgtcatcgtctaatggagttccaacaacctcaagaaaaccttcaggaacggggctatttagtttaatgattgattcatcaattgctaagaatagctcccataaagaggataatgataaatatgtctcgcccataaaggcagtaacatcggcctccaattctgcaagtagcaatatttccgaaattcctaaactaacactacctccacaagccaaaatcggtactgttattccaccgtcagagaatcaagttcccaatattaaaatcgagaatacagaagaggataataaaaggagtgacataacaaatgaaatctctgttaaaccaacttctagcattgctgataaactgaaacaatttgagcaaagttccgaaaagaaatcatcaccaaaggaaaatcctatagcaaaagaagaaatggattcaaaaccaaaactatccaataaatttatcacttcaatgaatgatgtgtccacagatgattcaagctctgatggtaacgaaaatgacgatgcagacgatgatgatgattttacctatatggcattgaaacaaacaatgaagagagaaggttcaaaaattgaaaaaaataatgacagcaaactacctgcaaatatagtggaacttgatttacatgagtcaccggagtccgtgaagattgaatctcctgaatcgataaaggaaatcacgtcatctgaaatgtcttcagaaatgccaagtagttcgctgcctaagagattagtagaggatgttgagccttcagaaatgccagagaagggcgcatctgtagaatcagtcaggaagaaaaattttcaagaaccacttggtaatgtcgaatctccggatatgacgcagaaggtcaagtctttgggtatgacaggaaaggctgtaggcccagaatcagatagtagggtcgaatctccgggcatgacaggacagattaaatctttgaatatggcaggaaaagttgtaggcccagaagcagatagtagggtcgaatctccgggcatgaaagagcagattaagtctttgggtatgacaggaaaaattacagctcaagaatcaatcaagtccccggaagcggctaggaagttggcgtcatcaggagaagttgacaaaattgaatctccaagaatggtaagggaaagcgagtccttggaggcagtaggcaatactatcccctcaaacatgacagtgaaaatggaatccccaaatttaaagggaaatactgtgtctgaacctcaagaaataaggagagacattgcctcctcagagccgatagagaatgttgaccccccaaaagtactaaaaaagattgtctttccaaaggctgttaatagaactggatcaccaaaatcagtcgaaaagactccatcttcagctacactgaaaaagagcgggctcccagaaccgaatagccaaattgtttctcctgaattggcaaaaaattcacctctagcaccgataaagaaaaatgtcgagttacgagaaactaataaaccacatactgagactatcacttctgtggaaccaacaaacaaggatgccaatacttcttggagagacgccgacttaaaccgtacgatcaaacgagaggaggaggacgaagattttgatagagtgaaccacaatatccagatcactggtgcatatacgaaaactggaaaaattgattatcataaaatacctgttgatcgtaaagcaaaaagcgaagcagaagtgcatacttccgaggaagatattgatgaatcaaataatgttaatggaaaaagagctgatgcccaaatacacatcactgaaagaaagcatgctttcgtaaatccaactgaaaattcacaggtaaaaaagacgagccactcaccatttttaaacagtaaaccggttcaatacgagaactcagagtcgaacggcggcattaacaaccacataaagataaaaaatactggagaaactacggcacatgacgagaaacattatagtgatgatgatgattctagctatcaatttgttcccatgaaacatgaagaacaagaacaagaacaaaacagaagtgaggaagaggaaagtgaagatgacgatgaagaggaagaagacagtgattttgatgtggatacatttgacattgaaaatccggataatacactatcagagttactattgtatttagaacatcaaacaatggacgtcatatccacgattcaatcgcttttgacatcgatcaagaaaccacaggtgacgaagggtaatttgaggggagaatcgaatgcaatcaaccaagtcataggtcaaatggtggacgctactagcatatcaatggagcaaagcagaaatgccaatttgaagaaacacggtgattgggtggtgcaaagtctaagagactgttcgcgtagaatgacaattttgtgccaattaactggcgatggaatactagcgaaggaaaagagcgatcaagattatgctgacaaaaacttcaaacagcggttggcagggattgcgtttgatgttgccaaatgtacaaaggagctggtaaaaactgtagaagaggcaagtttgaaggacgaaataaattatttgaattcgaagttgaagtaa

SEQ ID NO: 9 - DNA, 1605 bp, Saccharomyces cerevisiae
  source 1..1605: mol_type = genomic DNA
atgggtacgtcaagcgaggtttctctcgcacatcatagagatatcttccattactacgtctcactgaagacttttttcgaggtgactggcgaaaatcgtgacaggtcaaattcgacacgagctcaaaaggccagagccaagctgttgaagctatcttcttcgcaattttacgagctgagtacagacgtgtccgatgagctgcagaggagaatcggtgaagatgctaaccaaccagattaccttttgccgaaggcaaatttccacatgaaaaggaaccaggctagacagaaactggccaatctatcacaaactcgatttaatgatttgttggacgatatcctttttgagatcaagagaagagggttcgacaaggatttggatgctccacggcccccattaccgcagccgatgaaacaagaggtcagcaaagacagcgatgatactgcaagaacatccacaaattcttcctctgtgactcaagtagctccaaacgtctccgtacaaccttctttggtcattcctaagatggcatctatcgattggtcttctgaggaagaagaagaggagcaagtaaaggagaagccaaatgaaccggagggaaaacaaacaagcatggatgaaaagaaagaggctaaacctgctctaaaccccatagttacagattctgatctgcctgactcccaagtgctcgctcgtgatatcacatcaatggcaaggactccaacaacgacgcataaaaattactgggacgttaatgattctccaattatcaaggtagataaagatatcgataacgaaaagggtcccgaacagttgaagagccctgaagtacaacgggctgagaacaataaccctaactcagagatggaagacaaggttaaagaactgactgatttaaacagcgacttacatttgcaaattgaagatttgaatgctaagttagcatctttaaccagcgagaaggaaaaggagaagaaggaagagaaggaggaaaaagaaaaggaaaagaacttaaagattaactacaccattgatgaaagttttcagaaagaattgctgtcattaaactctcaaatcggtgaattatcaattgagaatgaaaatttgaagcagaaaatttcagaattcgaactgcatcaaaaaaagaatgacaaccataatgatttgaaaatcactgacggttttattagcaagtactcttctgccgatgggctcattccagctcaatacatcttaaacgctaacaacttgataattcaatttactactaggctttccgcagtacccataggcgactccacggcaatttcccatcaaattggcgaagagttatttcaaatattatcccagttatcgaacctaatctcccagctattactatcggccgacctattacagtacaaagatcaggtcattttactgaaggcatcattatcgcatgcgatcacatcgataagatatttctctgtttacggtcccgtattaattccgaaaataactgtgcaagctgctgtttcagaggtttgttttgccatgtgtaatctaattgattcagcgaaaataaaatccgattcaaatggtgagagcaccacctctaatgaaggt

SEQ ID NO: 10 - DNA, 762 bp, Saccharomyces cerevisiae
  source 1..762: mol_type = genomic DNA
aacaataaccctaactcagagatggaagacaaggttaaagaactgactgatttaaacagcgacttacatttgcaaattgaagatttgaatgctaagttagcatctttaaccagcgagaaggaaaaggagaagaaggaagagaaggaggaaaaagaaaaggaaaagaacttaaagattaactacaccattgatgaaagttttcagaaagaattgctgtcattaaactctcaaatcggtgaattatcaattgagaatgaaaatttgaagcagaaaatttcagaattcgaactgcatcaaaaaaagaatgacaaccataatgatttgaaaatcactgacggttttattagcaagtactcttctgccgatgggctcattccagctcaatacatcttaaacgctaacaacttgataattcaatttactactaggctttccgcagtacccataggcgactccacggcaatttcccatcaaattggcgaagagttatttcaaatattatcccagttatcgaacctaatctcccagctattactatcggccgacctattacagtacaaagatcaggtcattttactgaaggcatcattatcgcatgcgatcacatcgataagatatttctctgtttacggtcccgtattaattccgaaaataactgtgcaagctgctgtttcagaggtttgttttgccatgtgtaatctaattgattcagcgaaaataaaatccgattcaaatggtgagagcaccacctctaatgaaggt

SEQ ID NO: 11 - DNA, 441 bp, Saccharomyces cerevisiae
  source 1..441: mol_type = genomic DNA
ttgaaaatcactgacggttttattagcaagtactcttctgccgatgggctcattccagctcaatacatcttaaacgctaacaacttgataattcaatttactactaggctttccgcagtacccataggcgactccacggcaatttcccatcaaattggcgaagagttatttcaaatattatcccagttatcgaacctaatctcccagctattactatcggccgacctattacagtacaaagatcaggtcattttactgaaggcatcattatcgcatgcgatcacatcgataagatatttctctgtttacggtcccgtattaattccgaaaataactgtgcaagctgctgtttcagaggtttgttttgccatgtgtaatctaattgattcagcgaaaataaaatccgattcaaatggtgagagcaccacctctaatgaaggt

SEQ ID NO: 12 - DNA, 135 bp, Saccharomyces cerevisiae
  source 1..135: mol_type = genomic DNA
ttgaaaatcactgacggttttattagcaagtactcttctgccgatgggctcattccagctcaatacatcttaaacgctaacaacttgataattcaatttactactaggctttccgcagtacccataggcgactcc

SEQ ID NO: 13 - DNA, 60 bp, Saccharomyces cerevisiae
  source 1..60: mol_type = genomic DNA
caatacatcttaaacgctaacaacttgataattcaatttactactaggctttccgcagta

SEQ ID NO: 14 - protein, 19 aa, Saccharomyces cerevisiae
LKITDGFISKYSSADGLIP

SEQ ID NO: 15 - protein, 33 aa, Saccharomyces cerevisiae
SADGLIPAQYILNANNLIIQFTTRLSAVPIGDS

SEQ ID NO: 16 - DNA, 59 bp, synthetic construct
  source 1..59: mol_type = other DNA
cgaaaataaaatccgattcaaatggtgagagcaccacctcggatccccgggttaattaa

SEQ ID NO: 17 - DNA, 60 bp, synthetic construct
  source 1..60: mol_type = other DNA
ctttgtcttccttttctttctcctctagatactactaactgaattcgagctcgtttaaac

SEQ ID NO: 18 - DNA, 60 bp, synthetic construct
  source 1..60: mol_type = other DNA
tcccgaacagttgaagagccctgaagtacaacgggctgagcggatccccgggttaattaa

SEQ ID NO: 19 - DNA, 58 bp, synthetic construct
  source 1..58: mol_type = other DNA
aactacctgcaaatatagtggaacttgatttacatgagcggatccccgggttaattaa

SEQ ID NO: 20 - DNA, 56 bp, synthetic construct
  source 1..56: mol_type = other DNA
ccatcttcagctacactgaaaaagagcgggctcccacggatccccgggttaattaa

SEQ ID NO: 21 - DNA, 40 bp, synthetic construct
  source 1..40: mol_type = other DNA
gcggtggcggccgctctagagtatacatataatcacgtaa

SEQ ID NO: 22 - DNA, 40 bp, synthetic construct
  source 1..40: mol_type = other DNA
gtttactctttcttttcttttgtttattctgtttcggtgg

SEQ ID NO: 23 - DNA, 40 bp, synthetic construct
  source 1..40: mol_type = other DNA
aaagaaaagaaagagtaaacgagaacaataaccctaactc

SEQ ID NO: 24 - DNA, 40 bp, synthetic construct
  source 1..40: mol_type = other DNA
ggatccgtcgacctgcagcggaggtggtgctctcaccatt

SEQ ID NO: 25 - DNA, 39 bp, synthetic construct
  source 1..39: mol_type = other DNA
attcaaatggtgagagcaccacctccgctgcaggtcgac

SEQ ID NO: 26 - DNA, 40 bp, synthetic construct
  source 1..40: mol_type = other DNA
gagttagggttattgttctcgtttactctttcttttcttt